SEQUENCE LISTING (23 sequences)

SEQ ID NO: 1 - DNA, 3464 bp, Homo sapiens
  source 1..3464: mol_type = other DNA; note = 191P4D12
  CDS 264..1796: protein_id = 2; translation = MPLSLGAEMWGPEAWLLLLLLLASFTGRCPAGELETSDVVTVVL GQDAKLPCFYRGDSGEQVGQVAWARVDAGEGAQELALLHSKYGLHVSPAYEGRVEQPP PPRNPLDGSVLLRNAVQADEGEYECRVSTFPAGSFQARLRLRVLVPPLPSLNPGPALE EGQGLTLAASCTAEGSPAPSVTWDTEVKGTTSSRSFKHSRSAAVTSEFHLVPSRSMNG QPLTCVVSHPGLLQDQRITHILHVSFLAEASVRGLEDQNLWHIGREGAMLKCLSEGQP PPSYNWTRLDGPLPSGVRVDGDTLGFPPLTTEHSGIYVCHVSNEFSSRDSQVTVDVLD PQEDSGKQVDLVSASVVVVGVIAALLFCLLVVVVVLMSRYHRRKAQQMTQKYEEELTL TRENSIRRLHSHHTDPRSQPEESVGLRAEGHPDSLKDNSSCSVMSEEPEGRSYSTLTT VREIETQTELLSPGSGRAEEEEDQDEGIKQAMNHFVQENGTLRAKPTGNGIYINGRGH LV
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  source 1..510: note = 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  source 1..1432: mol_type = other DNA; note = Ha22-2(2,4)6.1 heavy chain
  CDS 32..1432: protein_id = 4; translation = MELGLCWVFLVAILEGVQCEVQLVESGGGLVQPGGSLRLSCAAS GFTFSSYNMNWVRQAPGKGLEWVSYISSSSSTIYYADSVKGRFTISRDNAKNSLSLQM NSLRDEDTAVYYCARAYYYGMDVWGQGTTVTVSSASTKGPSVFPLAPSSKSTSGGTAA LGCLVKDYFPEPVTVSWNSGALTSGVHTFPAVLQSSGLYSLSSVVTVPSSSLGTQTYI CNVNHKPSNTKVDKRVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFSCSVMHEA LHNHYTQKSLSLSPGK
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  source 1..466: note = Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  source 1..735: mol_type = other DNA; note = Ha22-2(2,4)6.1 light chain
  CDS 25..735: protein_id = 6; translation = MDMRVPAQLLGLLLLWFPGSRCDIQMTQSPSSVSASVGDRVTIT CRASQGISGWLAWYQQKPGKAPKFLIYAASTLQSGVPSRFSGSGSGTDFTLTISSLQP EDFATYYCQQANSFPPTFGGGTKVEIKRTVAAPSVFIFPPSDEQLKSGTASVVCLLNN FYPREAKVQWKVDNALQSGNSQESVTEQDSKDSTYSLSSTLTLSKADYEKHKVYACEV THQGLSSPVTKSFNRGEC
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  source 1..236: note = Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  source 1..466: note = Ha22-2(2,4)6.1 heavy chain

SEQ ID NO: 8 - protein, 236 aa, Homo sapiens; identical to SEQ ID NO: 6
  source 1..236: note = Ha22-2(2,4)6.1 light chain

SEQ ID NO: 9 - protein, 5 aa, Homo sapiens
  source 1..5: note = CDR1 of heavy chain
SYNMN

SEQ ID NO: 10 - protein, 17 aa, Homo sapiens
  source 1..17: note = CDR2 of heavy chain
YISSSSSTIYYADSVKG

SEQ ID NO: 11 - protein, 8 aa, Homo sapiens
  source 1..8: note = CDR3 of heavy chain
AYYYGMDV

SEQ ID NO: 12 - protein, 11 aa, Homo sapiens
  source 1..11: note = CDR1 of light chain
RASQGISGWLA

SEQ ID NO: 13 - protein, 7 aa, Homo sapiens
  source 1..7: note = CDR2 of light chain
AASTLQS

SEQ ID NO: 14 - protein, 9 aa, Homo sapiens
  source 1..9: note = CDR3 of light chain
QQANSFPPT

SEQ ID NO: 15 - protein, 4 aa, synthetic construct
  source 1..4: note = linker
GFLG

SEQ ID NO: 16 - protein, 8 aa, synthetic construct
  source 1..8: note = VH CDR1 according to IMGT
GFTFSSYN

SEQ ID NO: 17 - protein, 8 aa, synthetic construct
  source 1..8: note = VH CDR2 according to IMGT
ISSSSSTI

SEQ ID NO: 18 - protein, 10 aa, synthetic construct
  source 1..10: note = VH CDR3 according to IMGT
ARAYYYGMDV

SEQ ID NO: 19 - protein, 6 aa, synthetic construct
  source 1..6: note = VL CDR1 according to IMGT
QGISGW

SEQ ID NO: 20 - skipped

SEQ ID NO: 21 - protein, 9 aa, synthetic construct
  source 1..9: note = VL CDR3 according to IMGT
QQANSFPPT

SEQ ID NO: 22 - protein, 117 aa, synthetic construct
  source 1..117: note = heavy chain variable region (VH), the 20th amino acid (glutamic acid) to the 136th amino acid (serine) of SEQ ID NO:7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, synthetic construct
  source 1..108: note = light chain variable region (VL), the 23rd amino acid (aspartic acid) to the 130th amino acid (arginine) of SEQ ID NO:8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR

(19) United States
(12) Patent Application Publication
(10) Pub. No.: US 2026/0000604 A1
(43) Pub. Date: Jan. 1, 2026

(54) PHARMACEUTICAL COMPOSITIONS COMPRISING ANTI-191P4D12 ANTIBODY DRUG CONJUGATES AND METHODS OF USE THEREOF

(71) Applicants: AGENSYS, INC., Northbrook, IL (US); SEAGEN INC., Bothell, WA (US)

(72) Inventors: Orla MCGARVEY, Basel (CH); Gayathri RATNASWAMY, Encino, CA (US); Yingqing SUN, San Diego, CA (US); Marie Rose VAN SCHRAVENDIJK, Seattle, WA (US)

(73) Assignees: AGENSYS, INC., Northbrook, IL (US); SEAGEN INC., Bothell, WA (US)

(21) Appl. No.: 19/057,775

(22) Filed: Feb. 19, 2025

Related U.S. Application Data
(63) Continuation of application No. 17/298,865, filed on Jun. 1, 2021, now Pat. No. 12,257,340.
(60) Provisional application No. 62/774,819, filed on Dec. 3, 2018.

(51) Int. Cl. A61K 9/00; A61K 38/07; A61K 39/395; A61K 47/02; A61K 47/12; A61K 47/54; A61K 47/68

(52) U.S. Cl. CPC A61K 9/0019; A61K 38/07; A61K 39/3955; A61K 47/02; A61K 47/12; A61K 47/545; A61K 47/68031; A61K 47/6849

(57) ABSTRACT

A pharmaceutical composition comprising an antibody drug conjugate comprising an antibody or antigen binding fragment thereof that binds to 191P4D12 conjugated to one or more units of monomethyl auri statin E (MMAE) and a pharmaceutically acceptable excipient comprising L-histidine, polysorbate-20 (TWEEN-20), and at least one of trehalose dihydrate and sucrose.

### CROSS REFERENCE TO RELATED APPLICATIONS

This application claims the benefit of priority to U.S. Provisional Patent Application No. 62/774,819, filed Dec. 3, 2018, the disclosure of which is incorporated by reference herein in its entirety.

### 1. FIELD

Provided herein are pharmaceutical compositions comprising anti-191P4D12 antibody drug conjugates. Methods of using the pharmaceutical compositions are also provided herein.

### 2. BACKGROUND

Drug substances are usually administered as part of a formulation in combination with one or more other agents that serve varied and specialized pharmaceutical functions. Pharmaceutical excipients have various functions and contribute to the pharmaceutical formulations in many different ways, e.g., solubilization, dilution, thickening, stabilization, preservation, coloring, flavoring, etc. Properties that may be considered when formulating an active drug substance include bioavailability, ease of manufacture, ease of administration, and stability of the dosage form. Due to the varying properties of active drug substances being formulated, dosage forms typically require pharmaceutical excipients that are uniquely tailored to the active drug substance in order to achieve advantageous physical and pharmaceutical properties.

Thus, a need exists as to pharmaceutical compositions of anti-191P4D12 antibody drug conjugates having advantageous physical and pharmaceutical properties. The present invention satisfies this need and provides related benefits.

### 3. SUMMARY

In one aspect, provided herein is a pharmaceutical composition comprising (a) an antibody drug conjugate comprising an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:7 and a light chain variable region comprising CDRs comprising the amino acid sequences of the CDRs of the light chain variable region set forth in SEQ ID NO:8; and (b) a pharmaceutically acceptable excipient comprising L-histidine, polysorbate-20 (TWEEN-20), and at least one of trehalose dihydrate and sucrose.

In some embodiments, the antibody or antigen binding fragment thereof comprises CDR H1 comprising an amino acid sequence of SEQ ID NO:9, CDR H2 comprising an amino acid sequence of SEQ ID NO:10, CDR H3 comprising an amino acid sequence of SEQ ID NO: 11; CDR L1 comprising an amino acid sequence of SEQ ID NO:12, CDR L2 comprising an amino acid sequence of SEQ ID NO:13, and CDR L3 comprising an amino acid sequence of SEQ ID NO: 14.

In some embodiments, the antibody or antigen binding fragment thereof comprises a heavy chain variable region comprising the amino acid sequence ranging from the 20th amino acid (glutamic acid) to the 136th amino acid (serine) of SEQ ID NO:7 and a light chain variable region comprising the amino acid sequence ranging from the 23rd amino acid (aspartic acid) to the 130th amino acid (arginine) of SEQ ID NO:8.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antigen binding fragment is an Fab, F(ab′)₂, Fv or scFv fragment.

In some embodiments, the antibody is a fully human antibody.

In some embodiments, the antibody or antigen binding fragment thereof is recombinantly produced.

In some embodiments, the antibody drug conjugate has the following structure:

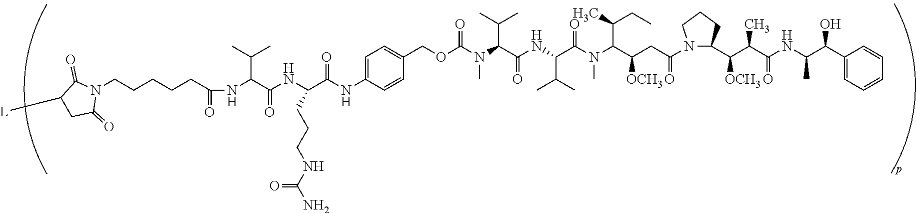

wherein L-represents the antibody or antigen binding fragment thereof and p is from 1 to 10.

In some embodiments, p is from 2 to 8.

In some embodiments, the antibody or antigen binding fragment is linked to each unit of monomethyl auristatin E (MMAE) via a linker.

In some embodiments, the linker is an enzyme-cleavable linker, and in one embodiment, the linker forms a bond with a sulfur atom of the antibody or antigen binding fragment thereof.

In some embodiments, the linker has a formula of: -Aₐ-W_{w}-Y_{y}—; wherein -A- is a stretcher unit, a is 0 or 1; −W— is an amino acid unit, w is an integer ranging from 0 to 12; and —Y— is a spacer unit, y is 0, 1, or 2.

In some embodiments, the stretcher unit has the structure of Formula (1) below; the amino acid unit is valine citrulline; and the spacer unit is a PAB group having the structure of Formula (2) below:

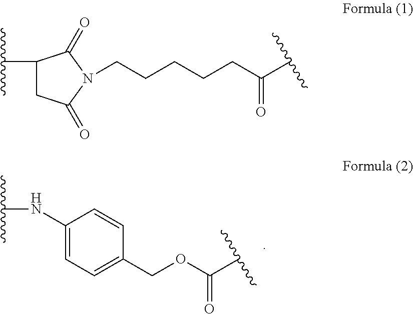 [SMILES: *C(=O)CCCCCN1C(=O)CC(*)C1=O ; *Nc1ccc(COC(*)=O)cc1]

In some embodiments, the stretcher unit forms a bond with a sulfur atom of the antibody or antigen binding fragment thereof; and wherein the spacer unit is linked to MMAE via a carbamate group.

In some embodiments, the antibody drug conjugate comprises from 1 to 10 units of MMAE per antibody or antigen binding fragment thereof.

In some embodiments, the antibody drug conjugate comprises from 2 to 8 units of MMAE per antibody or antigen binding fragment thereof.

In some embodiments, the pharmaceutical composition comprises the antibody drug conjugate at a concentration of from about 1 to about 20 mg/mL. In some embodiments, the pharmaceutical composition comprises the antibody drug conjugate at a concentration of from about 5 to about 15 mg/mL. In other embodiments, the pharmaceutical composition comprises the antibody drug conjugate at a concentration of from about 8 to about 12 mg/mL. In yet other embodiments, the pharmaceutical composition comprises the antibody drug conjugate at a concentration of about 10 mg/mL.

In some embodiments, L-histidine is present in the range of about 5 to about 50 mM. In other embodiments, L-histidine is present in the range of about 10 to about 40 mM. In other embodiments, L-histidine is present in the range of about 15 to about 35 mM. In other embodiments, L-histidine is present in the range of about 15 to about 30 mM. In other embodiments, L-histidine is present in the range of about 15 to about 25 mM. In yet other embodiments, L-histidine is present at about 20 mM.

In some embodiments, the concentration of TWEEN-20 is in the range of from about 0.001 to about 0.1% (v/v). In other embodiments, the concentration of TWEEN-20 is in the range of from about 0.0025 to about 0.075% (v/v). In other embodiments, the concentration of TWEEN-20 is in the range of from about 0.005 to about 0.05% (v/v). In other embodiments, the concentration of TWEEN-20 is in the range of from about 0.01 to about 0.03% (v/v). In yet other embodiments, the concentration of TWEEN-20 is in the range of about 0.02% (v/v).

In some embodiments, the pharmaceutical composition provided herein comprises trehalose dihydrate. In some embodiments, the trehalose dihydrate is present in the range of about 1 to about 20% (w/v). In some embodiments, the trehalose dihydrate is present in the range of about 2 to about 15% (w/v). In other embodiments, the trehalose dihydrate is present in the range of about 3 to about 10% (w/v). In yet other embodiments, the trehalose dihydrate is present in the range of about 4 to about 6% (w/v). In yet other embodiments, the trehalose dihydrate is present at about 5.5% (w/v).

In some embodiments, the trehalose dihydrate is present in the range of about 50 mM to about 300 mM. In some embodiments, the trehalose dihydrate is present in the range of about 75 mM to about 250 mM. In other embodiments, the trehalose dihydrate is present in the range of about 100 mM to about 200 mM. In yet other embodiments, the trehalose dihydrate is present in the range of about 130 mM to about 150 mM. In yet other embodiments, the trehalose dihydrate is present at about 146 mM.

In some embodiments, the pharmaceutical composition comprises sucrose. In some embodiments, the sucrose is present in the range of about 1 to about 20% (w/v). In some embodiments, the sucrose is present in the range of about 2 to about 15% (w/v). In other embodiments, the sucrose is present in the range of about 3 to about 10% (w/v). In other embodiments, the sucrose is present in the range of about 4 to about 6% (w/v). In yet other embodiments, the sucrose is present at about 5.5% (w/v).

In some embodiments, the sucrose is present in the range of about 50 mM to about 300 mM. In other embodiments, the sucrose is present in the range of about 75 mM to about 250 mM. In other embodiments, the sucrose is present in the range of about 100 mM to about 200 mM. In yet other embodiments, the sucrose is present in the range of about 130 mM to about 150 mM. In yet other embodiments, the sucrose is present at about 146 mM.

In some embodiments, the pharmaceutical composition has a pH in a range of about 5.5 to about 6.5. In some embodiments, the pharmaceutical composition has a pH in a range of about 5.7 to about 6.3. In other embodiments, the pharmaceutical composition has a pH of about 6.0.

In some embodiments, the pH is taken at room temperature. In some embodiments, the pH is taken at about 15° C. to about 27° C. In other embodiments, the pH is taken at about 4° C. In other embodiments, the pH is taken at about 25° C.

In some embodiments, the pharmaceutical composition provided herein comprises hydrochloric acid (HCl). In some embodiments, the pH is adjusted by HCl.

In other embodiments, the pharmaceutical composition provided herein comprises succinic acid. In some embodiments, the pH is adjusted by succinic acid.

In some embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In other embodiments, the pH is about 6.0 at 25° C.

In some specific embodiments, the pharmaceutical composition provided herein comprises:
(a) an antibody drug conjugate having the following structure:

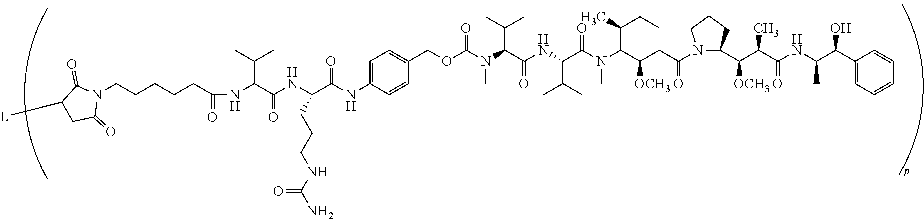

wherein L-represents the antibody or antigen binding fragment thereof and p is from 1 to 10; and (b) a pharmaceutically acceptable excipient comprising about 20 mM L-histidine, about 0.02% (w/v) TWEEN-20, about 5.5% (w/v) trehalose dihydrate, and HCl, wherein the pH is about 6.0 at 25° C.

In some embodiments, the antibody drug conjugate is at the concentration of about 10 mg/mL.

In other specific embodiments, the pharmaceutical composition provided herein comprises:
(a) an antibody drug conjugate comprising the following structure:

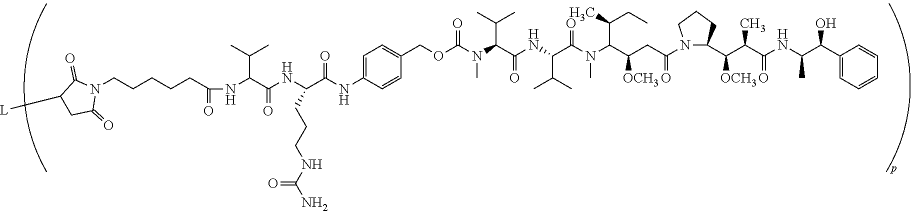

wherein L-represents the antibody or antigen binding fragment thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid, wherein the pH is about 6.0 at 25° C.
In some embodiments, the antibody drug conjugate is at the concentration of about 10 mg/mL in the pharmaceutical composition provided herein.

In yet other specific embodiments, the pharmaceutical composition provided herein comprises:
(a) an antibody drug conjugate comprising the following structure:

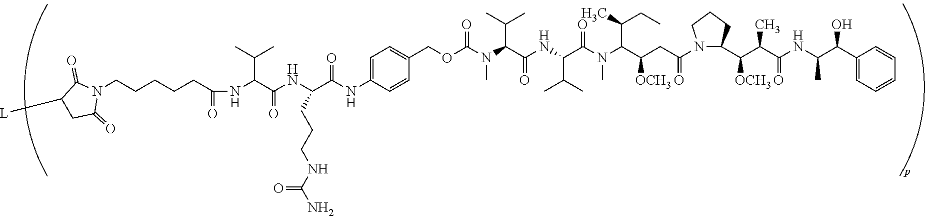

wherein L-represents the antibody or antigen binding fragment thereof and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the pH is about 6.0 at 25° C.

In some embodiments, the antibody drug conjugate is at the concentration of about 10 mg/mL in the pharmaceutical composition provided herein.

In some embodiments, the pharmaceutical composition provided herein is in a liquid form.

In other embodiments, the pharmaceutical composition provided herein is lyophilized.

In another aspect, provided herein is a lyophilized composition made by freeze-drying the pharmaceutical composition provided herein.

In some embodiments, the pharmaceutical composition is stored at −80° C., 4° C., 25° C. or 37° C.

In another aspect, provided herein is a method of preventing or treating a disease or disorder in a subject, comprising administering to the subject an effective amount of the pharmaceutical composition provided herein.

In some embodiments, the subject is a human subject.

In some embodiments, the cancer is a solid tumor. In some embodiments, the cancer is colon cancer, pancreatic cancer, ovarian cancer, lung cancer, bladder cancer, breast cancer, esophageal cancer, head cancer, or neck cancer.

In a specific embodiment, the cancer is colon cancer. In a specific embodiment, the cancer is pancreatic cancer. In a specific embodiment, the cancer is ovarian cancer. In a specific embodiment, the cancer is lung cancer. In some embodiments, the lung cancer is non-small cell lung cancer. In a specific embodiment, the cancer is bladder cancer. In a specific embodiment, the cancer is advanced bladder cancer. In a specific embodiment, the cancer is metastatic bladder cancer. In a specific embodiment, the cancer is breast cancer. In a specific embodiment, the cancer is esophageal cancer. In a specific embodiment, the cancer is head cancer. In a specific embodiment, the cancer is neck cancer. In a specific embodiment, the cancer has tumor cells expressing 191P4D12.

In some embodiments, the method provided herein further comprises administering to the subject a second therapeutic agent. In some embodiments, the second therapeutic agent is an immune checkpoint inhibitor. In some embodiments, the immune checkpoint inhibitor is a PD-1 inhibitor or a PD-L1 inhibitor. In other embodiments, the immune checkpoint inhibitor is a PD-1 inhibitor. In yet other embodiments, the PD-1 inhibitor is pembrolizumab or nivolumab. In other embodiments, the immune checkpoint inhibitor is a PD-L1 inhibitor. In other embodiments, the PD-L1 inhibitor is selected from a group consisting of atezolizumab, avelumab, and durvalumab.

In some embodiments, the antibody drug conjugate formulated in the pharmaceutical composition is administered at a dose of 1 to 10 mg/kg of the subject's body weight. In other embodiments, the antibody drug conjugate formulated in the pharmaceutical composition is administered at a dose of 1 to 5 mg/kg of the subject's body weight. In yet other embodiments, the antibody drug conjugate formulated in the pharmaceutical composition is administered at a dose of 1 to 2.5 mg/kg of the subject's body weight. In some embodiments, the antibody drug conjugate formulated in the pharmaceutical composition is administered at a dose of 1 to 1.25 mg/kg of the subject's body weight. In some embodiments, the antibody drug conjugate formulated in the pharmaceutical composition is administered at a dose of about 1 mg/kg of the subject's body weight. In some embodiments, the antibody drug conjugate formulated in the pharmaceutical composition is administered at a dose of about 1.25 mg/kg of the subject's body weight.

In some embodiments, the antibody drug conjugate formulated in the pharmaceutical composition is administered by an intravenous (IV) injection or infusion.

In some embodiments, the antibody drug conjugate formulated in the pharmaceutical composition is administered by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In other embodiments, the immune checkpoint inhibitor is atezolizumab, and wherein atezolizumab is administered at amount of about 1200 mg over about 60 minutes or 30 minutes.

In other embodiments, the antibody drug conjugate formulated in the pharmaceutical composition is administered by an intravenous (IV) injection or infusion over about 30 minutes three times every four-week cycle. In some embodiments, the antibody drug conjugate formulated in the pharmaceutical composition is administered by an intravenous (IV) injection or infusion over about 30 minutes on Days 1, 8 and 15 of every four-week cycle. In some embodiments, the method further comprises administering an immune checkpoint inhibitor by an intravenous (IV) injection or infusion. In some embodiments, the immune checkpoint inhibitor is pembrolizumab. In other embodiments, the immune checkpoint inhibitor is atezolizumab.

### 4. DESCRIPTION OF THE DRAWINGS

FIGS. 1A, 1B, 1C, and 1D depict the results of SDS-PAGE analysis for the 14-day stability study for the formulations F1-F14 at 40° C.

FIG. 1E depicts the summary of the PR-HPLC study described in Section 6.1.

FIGS. 1F, 1G, and 1H depicts the results of the SE-HPLC analysis for the formulations F1-F14 at 40° C.

FIG. 2A depicts the results of the shade study for the formulations F4, F9 and F14 at TO.

FIG. 2B depicts the SDS-PAGE results of the cycle freeze-thaw study for the formulations F4, F9, and F14.

FIG. 2C depicts the total cumulative counts per mL as measured by HIAC for the formulations F4, F9, and F14.

FIG. 3A depicts the results of the residual moisture analysis for the formulations F4, F9, and F14.

FIG. 3B depicts the A280 (concentration) results in the 12 week concurrent BDS and DP formulation study.

FIG. 3C depicts the A330 (turbidity) results in the 12 week concurrent BDS and DP formulation study.

FIG. 3D depicts the results of the SDS-PAGE analysis of the BDS (before lyophilization) at TO.

FIG. 3E depicts the results of the SDS-PAGE analysis of the BDS stored at −70° C. or 2-8° C. for 12 weeks.

FIG. 3F depicts the results of the SDS-PAGE analysis of the DP after lyophilization and reconstitution at T0.

FIG. 3G depicts the results of the SDS-PAGE analysis of the DP (after lyophilization and reconstitution) stored at 25° C. or 40° C. for 12 weeks.

FIG. 3H depicts the results of the SDS-PAGE analysis of the DP (after lyophilization and reconstitution) stored at 2-8° C. for 12 weeks.

FIG. 3I depicts the results of the SE-HPLC analysis for the AGS-22M6E BDS stored at 2-8° C. and −70° C. and lyophilized AGS-22M6E stored at 2-8° C., 25° C./60% RH and 40° C./75% RH conditions for 12 Weeks.

FIG. 4 depicts the results of the SDS-PAGE analysis for lyophilized formulation of F4 at both 3.0 and 1.5 mL fill volumes.

FIG. 5A depicts the nucleotide and amino acid sequences of 191P4D12 protein.

FIG. 5B depicts the nucleotide and amino acid sequences of the heavy chain and light chain of Ha22-2(2.4)6.1.

FIG. 5C depicts the amino acid sequences of the heavy chain and light chain of Ha22-2(2.4)6.1.

### 5. DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F (ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50:98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet®QK384 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®QK384, the Biacore®TM-2000, or the Biacore®TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1:755-68). Also available for the preparation of human monoclonal antibodies are methods described in Cole et al., Monoclonal Antibodies and Cancer Therapy 77 (1985); Boerner et al., 1991, J. Immunol. 147 (1): 86-95; and van Dijk and van de Winkel, 2001, Curr. Opin. Pharmacol. 5:368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6 (5): 561-66; Brüggemann and Taussing, 1997, Curr. Opin. Biotechnol. 8 (4): 455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and γ chains and four CH domains for u and & isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while u and & contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27 (1): 55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309:657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below.

TABLE 30

| | Kabat | AbM | Chothia | Contact | IMGT |
|---|---|---|---|---|---|
| CDR L1 | L24--L34 | L24--L34 | L24--L34 | L30--L36 | L27--L38 |
| CDR L2 | L50--L56 | L50--L56 | L50--L56 | L46--L55 | L56--L65 |
| CDR L3 | L89--L97 | L89--L97 | L89--L97 | L89--L96 | L105-- |
| | | | | | L117 |
| CDR H1 | H31--H35B | H26-- | H26-- | H30-- | H27--H38 |
| | (Kabat | H35B | H32 . . .34 | H35B | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H52--H56 | H47--H58 | H56--H65 |
| CDR H3 | H95--H102 | H95-- | H95-- | H93-- | H105- |
| | | H102 | H102 | H101 | H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well-known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well-known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well-known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is 5′ end; the left-hand direction of double-stranded polynucleotide sequences is referred to as 5′ direction. The direction of 5′ to 3′ addition of nascent RNA transcripts is referred to as the transcription direction; sequence regions on the DNA strand comprising the same sequence as the RNA transcript that are 5′ to 5′ end of the RNA transcript are referred to as “upstream sequences”; sequence regions on the DNA strand comprising the same sequence as the RNA transcript that are 3′ to the 3′ end of the RNA transcript are referred to as “downstream sequences.”

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as 1-histidine, glycine, glutamine, asparagine, arginine, or lysine; monosaccharides, disaccharides, and other carbohydrates, including glucose, sucrose, trehalose dihydrate, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. A excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like.

Compositions, including pharmaceutical compounds, may contain a binding molecule (e.g., an antibody), for example, in isolated or purified form, together with a suitable amount of excipients.

The abbreviation “MMAE” refers to monomethyl auristatin E.

Unless otherwise noted, the term “alkyl” refers to a saturated straight or branched hydrocarbon comprising from about 1 to about 20 carbon atoms (and all combinations and subcombinations of ranges and specific numbers of carbon atoms therein), with from about 1 to about 8 carbon atoms being preferred. Examples of alkyl groups are methyl, ethyl, n-propyl, iso-propyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. Alkyl groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl, and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more groups including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the terms “alkenyl” and “alkynyl” refer to straight and branched carbon chains comprising from about 2 to about 20 carbon atoms (and all combinations and subcombinations of ranges and specific numbers of carbon atoms therein), with from about 2 to about 8 carbon atoms being preferred. An alkenyl chain has at least one double bond in the chain and an alkynyl chain has at least one triple bond in the chain. Examples of alkenyl groups include, but are not limited to, ethylene or vinyl, allyl, -1-butenyl, -2-butenyl, -isobutylenyl, -1-pentenyl, -2-pentenyl, -3-methyl-1-butenyl, -2-methyl-2-butenyl, and -2,3-dimethyl-2-butenyl. Examples of alkynyl groups include, but are not limited to, acetylenic, propargyl, acetylenyl, propynyl, -1-butynyl, -2-butynyl, -1-pentynyl, -2-pentynyl, and -3-methyl-1 butynyl. Alkenyl and alkynyl groups, whether alone or as part of another group, can be optionally substituted with one or more groups, preferably 1 to 3 groups (and any additional substituents selected from halogen), including but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkyenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkylene” refers to a saturated branched or straight chain hydrocarbon radical comprising from about 1 to about 20 carbon atoms (and all combinations and subcombinations of ranges and specific numbers of carbon atoms therein), with from about 1 to about 8 carbon atoms being preferred and having two monovalent radical centers derived by the removal of two hydrogen atoms from the same or two different carbon atoms of a parent alkane. Typical alkylenes include, but are not limited to, methylene, ethylene, propylene, butylene, pentylene, hexylene, heptylene, ocytylene, nonylene, decalene, 1,4-cyclohexylene, and the like. Alkylene groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)2, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkenylene” refers to an optionally substituted alkylene group containing at least one carbon-carbon double bond. Exemplary alkenylene groups include, for example, ethenylene (—CH═CH—) and propenylene (—CH≡CHCH₂—).

Unless otherwise noted, the term “alkynylene” refers to an optionally substituted alkylene group containing at least one carbon-carbon triple bond. Exemplary alkynylene groups include, for example, acetylene (—C≡C—), propargyl (—CH₂C≡C—), and 4-pentynyl (—CH₂CH₂CH₂C≡CH—).

Unless otherwise noted, the term “aryl” refers to a monovalent aromatic hydrocarbon radical of 6-20 carbon atoms (and all combinations and subcombinations of ranges and specific numbers of carbon atoms therein) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, phenyl, naphthalene, anthracene, biphenyl, and the like.

An aryl group, whether alone or as part of another group, can be optionally substituted with one or more, preferably 1 to 5, or even 1 to 2 groups including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)2, —NHC(O)R′, —SR′, —SO₃R′, —S(O)₂R′, —S(O)R′, —OH, —NO₂, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “arylene” refers to an optionally substituted aryl group which is divalent (i.e., derived by the removal of two hydrogen atoms from the same or two different carbon atoms of a parent aromatic ring system) and can be in the ortho, meta, or para configurations as shown in the following structures with phenyl as the exemplary aryl group.

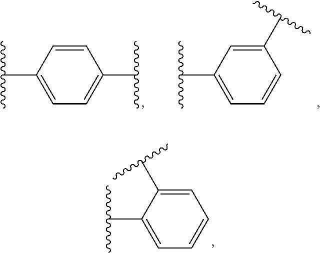

Typical “—(C₁-C₈ alkylene)aryl,” “—(C₂-C₈ alkenylene)aryl”, “and —(C₂-C₈ alkynylene)aryl” groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

Unless otherwise noted, the term “heterocycle,” refers to a monocyclic, bicyclic, or polycyclic ring system having from 3 to 14 ring atoms (also referred to as ring members) wherein at least one ring atom in at least one ring is a heteroatom selected from N, O, P, or S (and all combinations and subcombinations of ranges and specific numbers of carbon atoms and heteroatoms therein). The heterocycle can have from 1 to 4 ring heteroatoms independently selected from N, O, P, or S. One or more N, C, or S atoms in a heterocycle can be oxidized. A monocylic heterocycle preferably has 3 to 7 ring members (e.g., 2 to 6 carbon atoms and 1 to 3 heteroatoms independently selected from N, O, P, or S), and a bicyclic heterocycle preferably has 5 to 10 ring members (e.g., 4 to 9 carbon atoms and 1 to 3 heteroatoms independently selected from N, O, P, or S). The ring that includes the heteroatom can be aromatic or non-aromatic. Unless otherwise noted, the heterocycle is attached to its pendant group at any heteroatom or carbon atom that results in a stable structure. Heterocycles are described in Paquette, “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 82:5566 (1960). Examples of “heterocycle” groups include by way of example and not limitation pyridyl, dihydropyridyl, tetrahydropyridyl (piperidyl), thiazolyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, O-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl. Preferred “heterocycle” groups include, but are not limited to, benzofuranyl, benzothiophenyl, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A heterocycle group, whether alone or as part of another group, can be optionally substituted with one or more groups, preferably 1 to 2 groups, including but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or aryl.

By way of example and not limitation, carbon-bonded heterocycles can be bonded at the following positions: position 2, 3, 4, 5, or 6 of a pyridine; position 3, 4, 5, or 6 of a pyridazine; position 2, 4, 5, or 6 of a pyrimidine; position 2, 3, 5, or 6 of a pyrazine; position 2, 3, 4, or 5 of a furan, tetrahydrofuran, thiofuran, thiophene, pyrrole or tetrahydropyrrole; position 2, 4, or 5 of an oxazole, imidazole or thiazole; position 3, 4, or 5 of an isoxazole, pyrazole, or isothiazole; position 2 or 3 of an aziridine; position 2, 3, or 4 of an azetidine; position 2, 3, 4, 5, 6, 7, or 8 of a quinoline; or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles can be bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole; position 2 of a isoindole, or isoindoline; position 4 of a morpholine; and position 9 of a carbazole, or P-carboline. Still more typically, nitrogen bonded heterocycles include 1-aziridyl, 1-azetedyl, 1-pyrrolyl, 1-imidazolyl, 1-pyrazolyl, and 1-piperidinyl.

Unless otherwise noted, the term “carbocycle,” refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles preferably have 3 to 6 ring atoms, still more preferably 5 or 6 ring atoms. Bicyclic carbocycles preferably have 7 to 12 ring atoms, e.g., arranged as a bicyclo[4,5], [5,5], [5,6] or [6,6] system, or 9 or 10 ring atoms arranged as a bicyclo[5,6] or [6,6] system. The term “carbocycle” includes, for example, a monocyclic carbocycle ring fused to an aryl ring (e.g., a monocyclic carbocycle ring fused to a benzene ring). Carbocyles preferably have 3 to 8 carbon ring atoms. Carbocycle groups, whether alone or as part of another group, can be optionally substituted with, for example, one or more groups, preferably 1 or 2 groups (and any additional substituents selected from halogen), including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Examples of monocyclic carbocylic substituents include -cyclopropyl, -cyclobutyl, -cyclopentyl, -1-cyclopent-1-enyl, -1-cyclopent-2-enyl, -1-cyclopent-3-enyl, cyclohexyl, -1-cyclohex-1-enyl, -1-cyclohex-2-enyl, -1-cyclohex-3-enyl, -cycloheptyl, -cyclooctyl. -1,3-cyclohexadienyl, -1,4-cyclohexadienyl, -1,3-cycloheptadienyl, -1,3,5-cycloheptatrienyl, and -cyclooctadienyl.

A “carbocyclo,” whether used alone or as part of another group, refers to an optionally substituted carbocycle group as defined above that is divalent (i.e., derived by the removal of two hydrogen atoms from the same or two different carbon atoms of a parent carbocyclic ring system).

Unless otherwise indicated by context, a hyphen (-) designates the point of attachment to the pendant molecule. Accordingly, the term “—(C₁-C₈ alkylene)aryl” or “—C₁-C₈ alkylene(aryl)” refers to a C₁-C₈ alkylene radical as defined herein wherein the alkylene radical is attached to the pendant molecule at any of the carbon atoms of the alkylene radical and one of the hydrogen atoms bonded to a carbon atom of the alkylene radical is replaced with an aryl radical as defined herein.

When a particular group is “substituted”, that group may have one or more substituents, preferably from one to five substituents, more preferably from one to three substituents, most preferably from one to two substituents, independently selected from the list of substituents. The group can, however, generally have any number of substituents selected from halogen. Groups that are substituted are so indicated. It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Protective groups as used herein refer to groups which selectively block, either temporarily or permanently, one reactive site in a multifunctional compound. Suitable hydroxy-protecting groups for use in the present invention are pharmaceutically acceptable and may or may not need to be cleaved from the parent compound after administration to a subject in order for the compound to be active. Cleavage is through normal metabolic processes within the body. Hydroxy protecting groups are well-known in the art, see, Protective Groups in Organic Synthesis by T. W. Greene and P. G. M. Wuts (John Wiley & sons, 3^{rd} Edition) incorporated herein by reference in its entirety and for all purposes and include, for example, ether (e.g., alkyl ethers and silyl ethers including, for example, dialkylsilylether, trialkylsilylether, dialkylalkoxysilylether), ester, carbonate, carbamates, sulfonate, and phosphate protecting groups. Examples of hydroxy protecting groups include, but are not limited to, methyl ether; methoxymethyl ether, methylthiomethyl ether, (phenyldimethylsilyl)methoxymethyl ether, benzyloxymethyl ether, p-methoxybenzyloxymethyl ether, p-nitrobenzyloxymethyl ether, o-nitrobenzyloxymethyl ether, (4-methoxyphenoxy)methyl ether, guaiacolmethyl ether, t-butoxymethyl ether, 4-pentenyloxymethyl ether, siloxymethyl ether, 2-methoxyethoxymethyl ether, 2,2,2-trichloroethoxymethyl ether, bis(2-chloroethoxy)methyl ether, 2-(trimethylsilyl)ethoxymethyl ether, menthoxymethyl ether, tetrahydropyranyl ether, 1-methoxycylcohexyl ether, 4-methoxytetrahydrothiopyranyl ether, 4-methoxytetrahydrothiopyranyl ether S,S-Dioxide, 1-[(2-choro-4-methyl)phenyl]-4-methoxypiperidin-4-yl ether, 1-(2-fluorophneyl)-4-methoxypiperidin-4-yl ether, 1,4-dioxan-2-yl ether, tetrahydrofuranyl ether, tetrahydrothiofuranyl ether; substituted ethyl ethers such as 1-ethoxyethyl ether, 1-(2-chloroethoxy)ethyl ether, 1-[2-(trimethylsilyl)ethoxy]ethyl ether, 1-methyl-1-methoxyethyl ether, 1-methyl-1-benzyloxyethyl ether, 1-methyl-1-benzyloxy-2-fluoroethyl ether, 1-methyl-iphenoxyethyl ether, 2-trimethylsilyl ether, t-butyl ether, allyl ether, propargyl ethers, p-chlorophenyl ether, p-methoxyphenyl ether, benzyl ether, p-methoxybenzyl ether 3,4-dimethoxybenzyl ether, trimethylsilyl ether, triethylsilyl ether, tripropylsilylether, dimethylisopropylsilyl ether, diethylisopropylsilyl ether, dimethylhexylsilyl ether, t-butyldimethylsilyl ether, diphenylmethylsilyl ether, benzoylformate ester, acetate ester, chloroacetate ester, dichloroacetate ester, trichloroacetate ester, trifluoroacetate ester, methoxyacetate ester, triphneylmethoxyacetate ester, phenylacetate ester, benzoate ester, alkyl methyl carbonate, alkyl 9-fluorenylmethyl carbonate, alkyl ethyl carbonate, alkyl 2,2,2,-trichloroethyl carbonate, 1,1,-dimethyl-2,2,2-trichloroethyl carbonate, alkylsulfonate, methanesulfonate, benzylsulfonate, tosylate, methylene acetal, ethylidene acetal, and t-butylmethylidene ketal. Preferred protecting groups are represented by the formulas —R^{a}, —Si(R^{a})(R^{a})(R^{a}), —C(O)R^{a}, —C(O)OR^{a}, —C(O)NH(R^{a}), —S(O)₂R^{a}, —S(O)₂OH, P(O)(OH)₂, and —P(O)(OH)OR^{a}, wherein R^{a} is C₁-C₂₀ alkyl, C₂-C₂₀ alkenyl, C₂-C₂₀ alkynyl, —C₁-C₂₀ alkylene(carbocycle), —C₂-C₂₀ alkenylene(carbocycle), —C₂-C₂₀ alkynylene(carbocycle), —C₆-C₁₀ aryl, —C₁-C₂₀ alkylene(aryl), —C₂-C₂₀ alkenylene(aryl), —C₂-C₂₀ alkynylene(aryl), —C₁-C₂₀ alkylene(heterocycle), —C₂-C₂₀ alkenylene(heterocycle), or —C₂-C₂₀ alkynylene(heterocycle) wherein said alkyl, alkenyl, alkynyl, alkylene, alkenylene, alkynylene, aryl, carbocycle, and heterocycle radicals whether alone or as part of another group are optionally substituted.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2 Pharmaceutical Compositions

In one aspect, provided herein are “pharmaceutical compositions,” which include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions may be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In some aspects, the pharmaceutical compositions may further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds may be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 below.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM. In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments, the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises
(a) an antibody drug conjugate comprising the following structure:

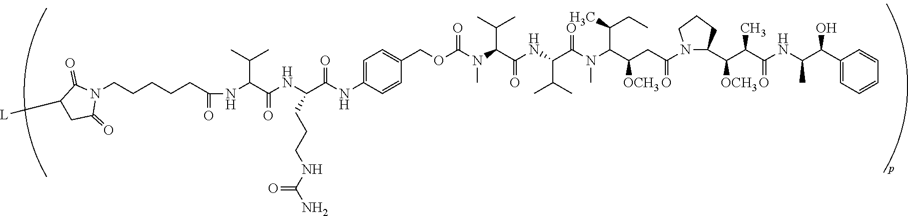

wherein L- represents the antibody or antigen binding fragment thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:
(a) an antibody drug conjugate comprising the following structure:

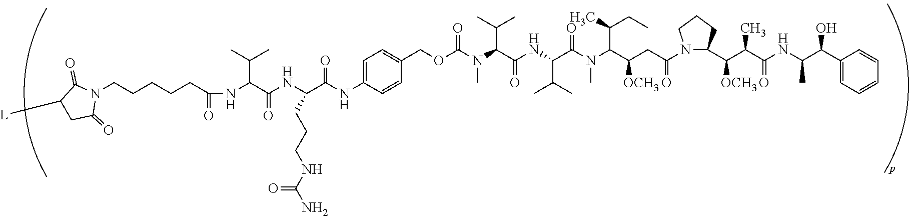

wherein L- represents the antibody or antigen binding fragment thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid,
wherein the antibody drug conjugate is at the concentration of about 10 mg/mL, and wherein the pH is about 6.0 at 25° C.

In yet another specific embodiment, the pharmaceutical composition provided herein comprises:
(a) an antibody drug conjugate comprising the following structure:

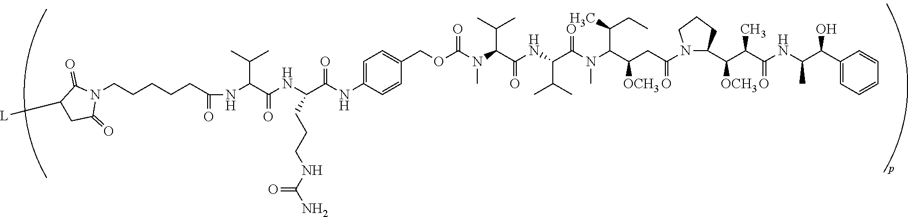

wherein L- represents the antibody or antigen binding fragment thereof and p is from 1 to 10; and
(b) a pharmaceutically acceptable excipient comprising about 20 mM L-histidine, about 0.02% (w/v) TWEEN-20, about 5.0% (w/v) sucrose, and HCl,
wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated. Other exemplary pharmaceutical compositions are provided in the Experimental section below.

A primary solvent in a vehicle may be either aqueous or non-aqueous in nature. In addition, the vehicle may contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein may contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition may be stored in a sterile vial as a solution, suspension, gel, emulsion, solid, or dihydrated or lyophilized powder. Such compositions may be stored either in a ready to use form, a lyophilized form requiring reconstitution prior to use, a liquid form requiring dilution prior to use, or other acceptable form. In some embodiments, a pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver peptides and the other agents described herein, including implants (e.g., implantable pumps) and catheter systems, both of which are known to the skilled artisan. Depot injections, which are generally administered subcutaneously or intramuscularly, may also be utilized to release peptides and/or other agents described herein over a defined period of time. Depot injections are usually either solid- or oil-based and generally comprise at least one of the formulation components set forth herein. The skilled artisan is familiar with possible formulations and uses of depot injections. In certain embodiments, the use of Nano Precision Medical's depot delivery technology (Nano Precision Medical; Emeryville, CA) is contemplated. The technology utilizes a titania nanotube membrane that produces zero-order release rates of macromolecules, such as protein and peptide therapeutics. The biocompatible membrane is housed in a small, subcutaneous implant that provides long-term (e.g., up to one year), constant-rate delivery of therapeutic macromolecules.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions may be in the form of a sterile injectable aqueous or oleagenous suspension. This suspension may be formulated using suitable dispersing or wetting agents and suspending agents disclosed herein or known to the skilled artisan. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration may include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-p-cyclodextrin, sulfobutylether-β-cyclodextrin, and sulfobutylether 7-O-cyclodextrin (CAPTISOL®, CyDex, Lenexa, KS).

In one embodiment, the pharmaceutical compositions provided herein may be formulated for single or multiple dosage administration. The single dosage formulations are packaged in an ampoule, a vial, or a syringe. The multiple dosage parenteral formulations may contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein may be formulated as immediate or modified release dosage forms, including delayed-, sustained, pulsed-, controlled, targeted-, and programmed-release forms.

In one embodiment, the pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

In one embodiment, suitable inner matrixes include polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

In one embodiment, suitable outer polymeric membranes include polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyloxyethanol copolymer, and ethylene/vinyl acetate/vinyl alcohol terpolymer.

Aqueous suspensions contain the active materials in admixture with excipients suitable for the manufacture thereof. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions provided herein may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein may be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They may also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

### 5.3 Anti-191P4D12 Antibody Drug Conjugate

The pharmaceutical compositions, formulations and dosage forms provided herein comprise anti-191P4D12 antibody drug conjugates. The anti-191P4D12 antibody drug conjugate provided herein comprises an antibody or antigen binding fragment thereof that binds to 191P4D12 conjugated to one or more units of cytotoxic agents (or drug units). The cytotoxic agents (or drug units) can be covalently linked directly or via a linker unit (LU).

In some embodiments, the antibody drug conjugate compound has the following formula:

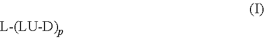

or a pharmaceutically acceptable salt or solvate thereof; wherein:
L is the antibody unit, e.g., the anti-191P4D12 antibody or an antigen binding fragment thereof as provided in Section 5.3.1 below, and
(LU-D) is a linker unit-drug unit moiety, wherein:
LU- is a linker unit, and
D is a drug unit having cytostatic or cytotoxic activity against a target cell; and
p is an integer from 1 to 20.

In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10.

In some embodiments, the antibody drug conjugate compound has the following formula:

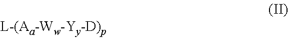

or a pharmaceutically acceptable salt or solvate thereof, wherein:
L is the Antibody unit, e.g., the anti-191P4D12 antibody or an antigen binding fragment thereof as provided in Section 5.3.1 below; and
-Aₐ-Ww-Y_{y}— is a linker unit (LU), wherein:
-A- is a stretcher unit,
a is 0 or 1,
each —W— is independently an amino acid unit,
w is an integer ranging from 0 to 12,
—Y— is a self-immolative spacer unit,
y is 0, 1 or 2;
D is a drug units having cytostatic or cytotoxic activity against the target cell; and
p is an integer from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 8, 2 to 7, 2 to 6, 2 to 5, 2 to 4 or 2 to 3. In other embodiments, p is 1, 2, 3, 4, 5 or 6. In some embodiments, p is 2 or 4. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

For compositions comprising a plurality antibodies or antigen binding fragments thereof, the drug loading is represented by p, the average number of drug molecules per antibody unit. Drug loading may range from 1 to 20 drugs (D) per antibody. The average number of drugs per antibody in preparation of conjugation reactions may be characterized by conventional means such as mass spectroscopy, ELISA assay, and HPLC. The quantitative distribution of antibody drug conjugates in terms of p may also be determined. In some instances, separation, purification, and characterization of homogeneous antibody drug conjugates where p is a certain value from antibody drug conjugates with other drug loadings may be achieved by means such as reverse phase HPLC or electrophoresis. In exemplary embodiments, p is from 2 to 8.

### 5.3.1 Anti-191P4D12 Antibodies or Antigen Binding Fragments

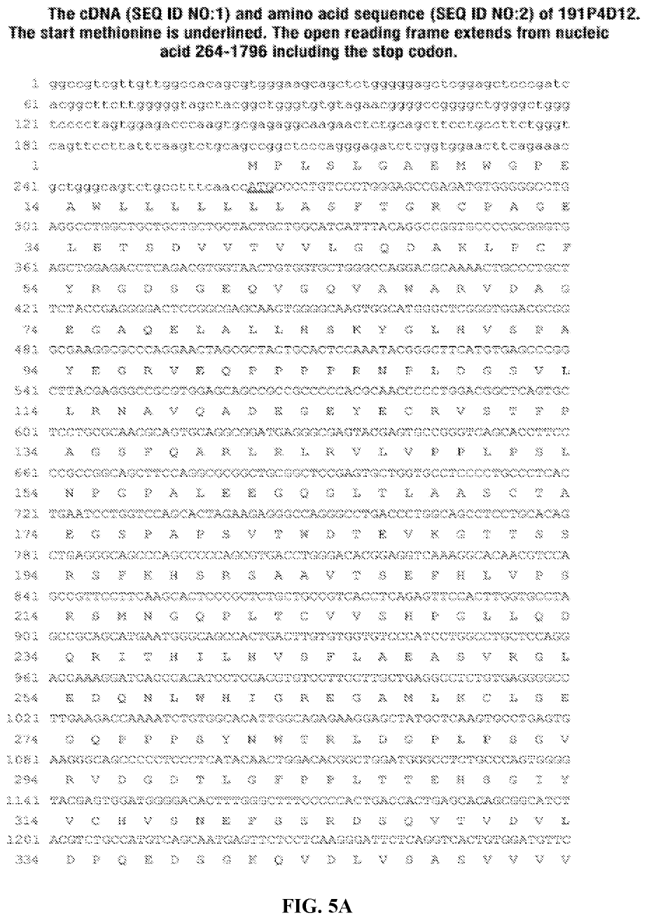

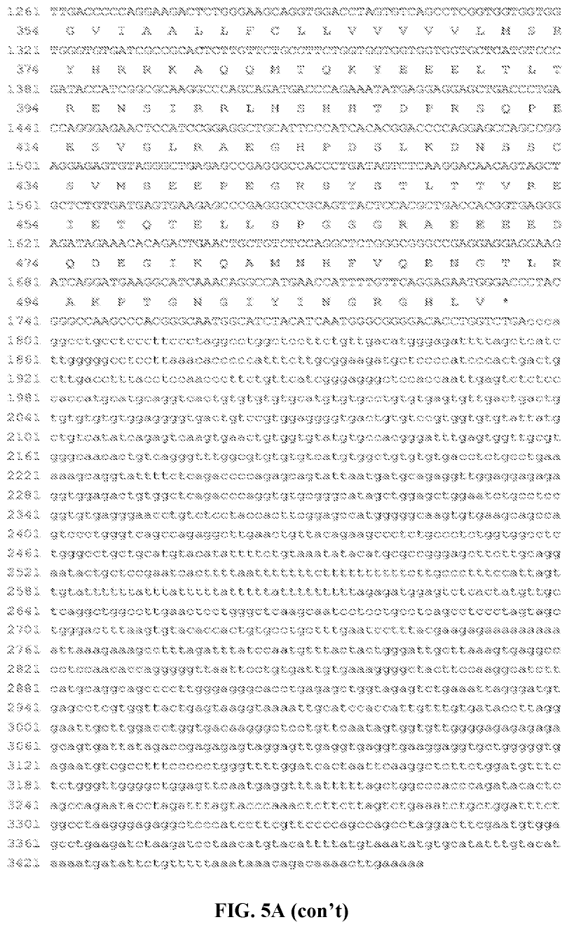

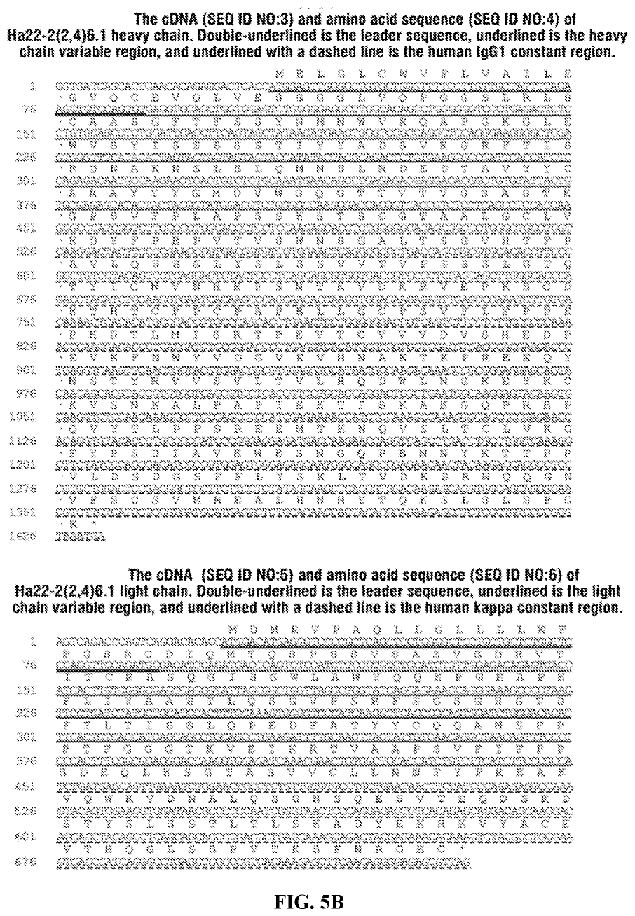

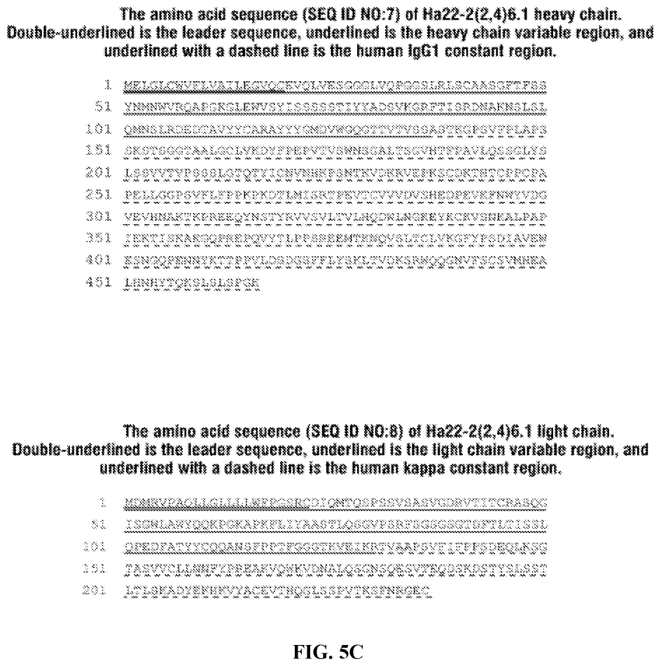

In one embodiment, the antibody or antigen binding fragment thereof that binds to 191P4D12-related proteins is an antibody or antigen binding fragment that specifically binds to 191P4D12 protein comprising amino acid sequence of SEQ ID NO:2 (see FIG. 5A). The corresponding cDNA encoding the 191P4D12 protein has a sequence of SEQ ID NO:1 (see FIG. 5A).

The antibody that specifically binds to 191P4D12 protein comprising amino acid sequence of SEQ ID NO:2 includes antibodies that can bind to other 191P4D12-related proteins. For example, antibodies that bind 191P4D12 protein comprising amino acid sequence of SEQ ID NO:2 can bind 191P4D12-related proteins such as 191P4D12 variants and the homologs or analogs thereof.

In some embodiments, the anti-191P4D12 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO: 6 (cDNA sequence of SEQ ID NO:5), as shown in FIG. 5B.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:7 and a light chain variable region comprising CDRs comprising the amino acid sequences of the CDRs of the light chain variable region set forth in SEQ ID NO:8. SEQ ID NO:7 and SEQ ID NO:8 are as shown in FIG. 5C and listed below:

SEQ ID NO: 7
MELGLCWVFLVAILEGVQCEVQLVESGGGLVQPGGSLRLSCAASGFTFSS
YNMNWVRQAPGKGLEWVSYISSSSSTIYYADSVKGRFTISRDNAKNSLSL
QMNSLRDEDTAVYYCARAYYYGMDVWGQGTTVTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEA
LHNHYTQKSLSLSPGK
SEQ ID NO: 8
MDMRVPAQLLGLLLLWFPGSRCDIQMTQSPSSVSASVGDRVTITCRASQG
ISGWLAWYQQKPGKAPKFLIYAASTLQSGVPSRFSGSGSGTDFTLTISSL
QPEDFATYYCQQANSFPPTFGGGTKVEIKRTVAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGEC

In some embodiments, the anti-191P4D12 antibody or antigen binding fragment thereof comprises a heavy chain variable region comprising the amino acid sequences of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising the amino acid sequences of the light chain variable region set forth in SEQ ID NO:23 (which is the amino acid sequence ranging from the 23rd amino acid (aspartic acid) to the 130th amino acid (arginine) of SEQ ID NO:8). In other embodiments, the anti-191P4D12 antibody or antigen binding fragment thereof comprises a heavy chain variable region consisting of the amino acid sequences of the heavy chain variable region set forth in SEQ ID NO:22 (which is the amino acid sequence ranging from the 20th amino acid (glutamic acid) to the 136th amino acid (serine) of SEQ ID NO:7) and a light chain variable region consisting of the amino acid sequences of the light chain variable region set forth in SEQ ID NO:23 (which is the amino acid sequence ranging from the 23rd amino acid (aspartic acid) to the 130th amino acid (arginine) of SEQ ID NO:8). SEQ ID NO: 22 and SEQ ID NO:23 are listed below:

SEQ ID NO: 22
EVQLVESGGGLVQPGGSLRLSCAASGFTFSSSSSYNMNWVRQAPGKGLEW
VSYISSTIYYADSVKGRFTISRDNAKNSLSLQMNSLRDEDTAVYYCARAY
YYGMDVWGQGTTVTVSS
SEQ ID NO: 23
DIQMTQSPSSVSASVGDRVTITCRASQGISGWLAWYQQKPGKAPKFLIYA
ASTLQSGVPSRFSGSGSGTDFTLTISSLQPEDFATYYCQQANSFPPTFGG
GTKVEIKR

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 30 above.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:7 according to Kabat numbering and a light chain variable region comprising CDRs comprising the amino acid sequences of the CDRs of the light chain variable region set forth in SEQ ID NO:8 according to Kabat numbering.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:7 according to AbM numbering and a light chain variable region comprising CDRs comprising the amino acid sequences of the CDRs of the light chain variable region set forth in SEQ ID NO:8 according to AbM numbering.

In other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:7 according to Chothia numbering and a light chain variable region comprising CDRs comprising the amino acid sequences of the CDRs of the light chain variable region set forth in SEQ ID NO:8 according to Chothia numbering.

In other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:7 according to Contact numbering and a light chain variable region comprising CDRs comprising the amino acid sequences of the CDRs of the light chain variable region set forth in SEQ ID NO:8 according to Contact numbering.

In yet other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:7 according to IMGT numbering and a light chain variable region comprising CDRs comprising the amino acid sequences of the CDRs of the light chain variable region set forth in SEQ ID NO:8 according to IMGT numbering.

As described above, the CDR sequences according to different numbering systems can be readily determined, e.g., using online tools such as the one provided by Antigen receptor Numbering And Receptor ClassificatIon (ANARCI). For example, the heavy chain CDR sequences within SEQ ID NO:7, and the light chain CDR sequences within SEQ ID NO:8 according to Kabat numbering as determined by ANARCI are listed in Table 31 below.

TABLE 31

| | VH of | VL of |
|---|---|---|
| | SEQ ID NO: 7 | SEQ ID NO: 8 |
| CDR1 | SYNMN | RASQGISGWLA |
| | (SEQ ID NO: 9) | (SEQ ID NO: 12) |
| CDR2 | YISSSSSTIYYADSVKG | AASTLQS |
| | (SEQ ID NO: 10) | (SEQ ID NO: 13) |
| CDR3 | AYYYGMDV | QQANSFPPT |
| | (SEQ ID NO: 11) | (SEQ ID NO: 14) |

For another example, the heavy chain CDR sequences within SEQ ID NO:22, and the light chain CDR sequences within SEQ ID NO:23 according to IMGT numbering as determined by ANARCI are listed in Table 32 below.

TABLE 32

| | VH of | VL of |
|---|---|---|
| | SEQ ID NO: 7 | SEQ ID NO: 8 |
| CDR1 | GFTFSSYN | QGISGW |
| | (SEQ ID NO: 16) | (SEQ ID NO: 19) |
| CDR2 | ISSSSSTI | AAS |
| | (SEQ ID NO: 17) | (SEQ ID NO: 20) |
| CDR3 | ARAYYYGMDV | QQANSFPPT |
| | (SEQ ID NO: 18) | (SEQ ID NO: 21) |

In some embodiments, the antibody or antigen binding fragment thereof comprises CDR H1 comprising an amino acid sequence of SEQ ID NO:9, CDR H2 comprising an amino acid sequence of SEQ ID NO:10, CDR H3 comprising an amino acid sequence of SEQ ID NO:11, CDR L1 comprising an amino acid sequence of SEQ ID NO:NO:12, CDR L2 comprising an amino acid sequence of SEQ ID NO:NO:13, and CDR L3 comprising an amino acid sequence of SEQ ID NO:NO:14.

In some embodiments, the antibody or antigen binding fragment thereof comprises a heavy chain variable region comprising the amino acid sequence ranging from the 20th amino acid (glutamic acid) to the 136th amino acid (serine) of SEQ ID NO:7 and a light chain variable region comprising the amino acid sequence ranging from the 23rd amino acid (aspartic acid) to the 130th amino acid (arginine) of SEQ ID NO:8.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also may be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In some embodiments, the antibody or antigen binding fragment provided herein comprises a heavy chain having more than 70% homologous to the heavy chain as set forth in SEQ ID NO:NO:7. In some embodiments, the antibody or antigen binding fragment provided herein comprises a heavy chain having more than 75% homologous to the heavy chain as set forth in SEQ ID NO:7. In some embodiments, the antibody or antigen binding fragment provided herein comprises a heavy chain having more than 80% homologous to the heavy chain as set forth in SEQ ID NO:7. In some embodiments, the antibody or antigen binding fragment provided herein comprises a heavy chain having more than 85% homologous to the heavy chain as set forth in SEQ ID NO:7. In some embodiments, the antibody or antigen binding fragment provided herein comprises a heavy chain having more than 90% homologous to the heavy chain as set forth in SEQ ID NO:7. In some embodiments, the antibody or antigen binding fragment provided herein comprises a heavy chain having more than 95% homologous to the heavy chain as set forth in SEQ ID NO:7.

In some embodiments, the antibody or antigen binding fragment provided herein comprises a light chain having more than 70% homologous to the light chain as set forth in SEQ ID NO:8. In some embodiments, the antibody or antigen binding fragment provided herein comprises a light chain having more than 75% homologous to the light chain as set forth in SEQ ID NO:8. In some embodiments, the antibody or antigen binding fragment provided herein comprises a light chain having more than 80% homologous to the light chain as set forth in SEQ ID NO:8. In some embodiments, the antibody or antigen binding fragment provided herein comprises a light chain having more than 85% homologous to the light chain as set forth in SEQ ID NO:8. In some embodiments, the antibody or antigen binding fragment provided herein comprises a light chain having more than 90% homologous to the light chain as set forth in SEQ ID NO:8. In some embodiments, the antibody or antigen binding fragment provided herein comprises a light chain having more than 95% homologous to the light chain as set forth in SEQ ID NO:8.

In some embodiments, the anti-191P4D12 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-191P4D12 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein: (a) the heavy chain variable region comprises CDRs comprising the amino acid sequences of the heavy chain variable region CDRs set forth in the antibody produced by a hybridoma deposited under the American Type Culture Collection (ATCC) Accession NO: PTA-11267; (b) the light chain variable region comprises CDRs comprising the amino acid sequences of the light chain variable region CDRs set forth in the antibody produced by a hybridoma deposited under the American Type Culture Collection (ATCC) Accession NO: PTA-11267.

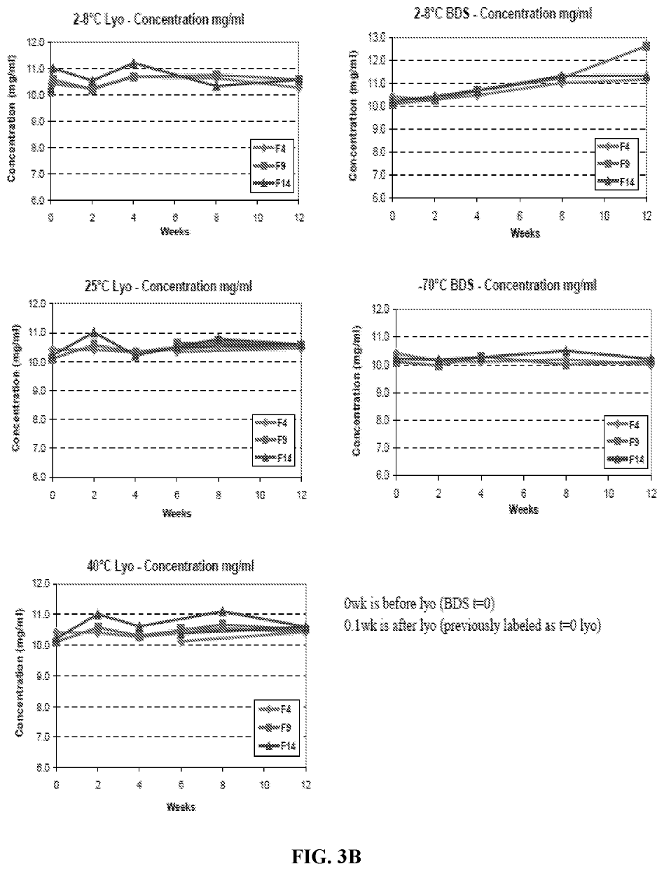

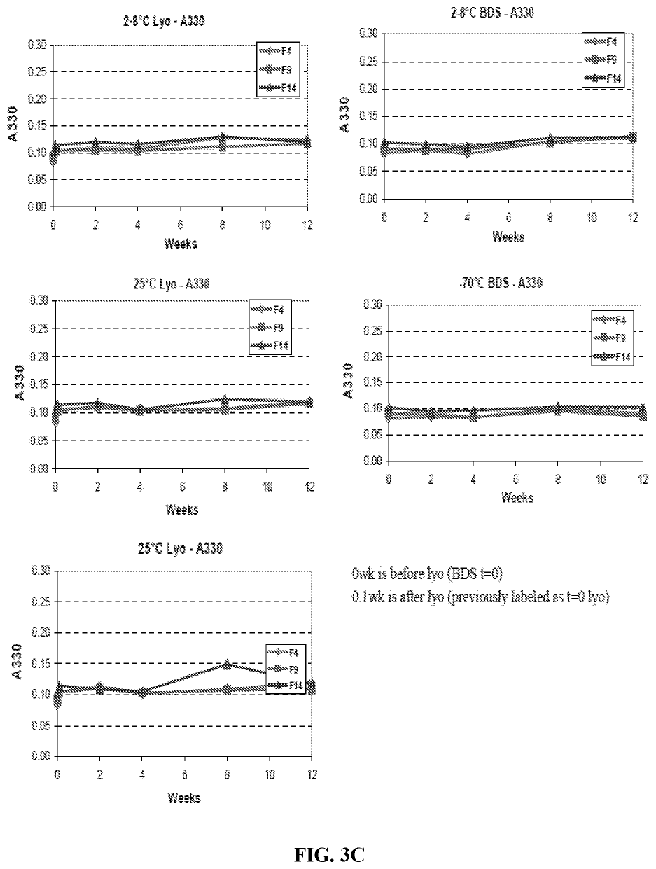

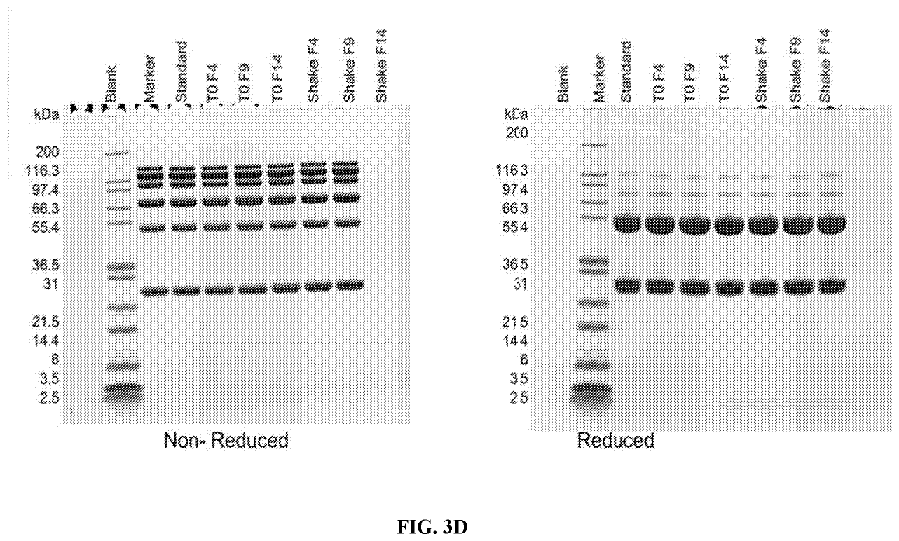

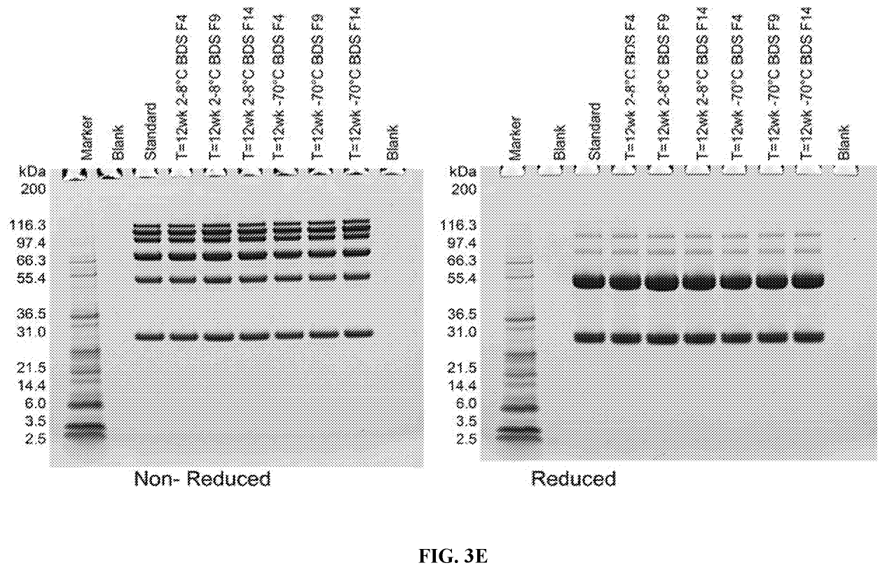

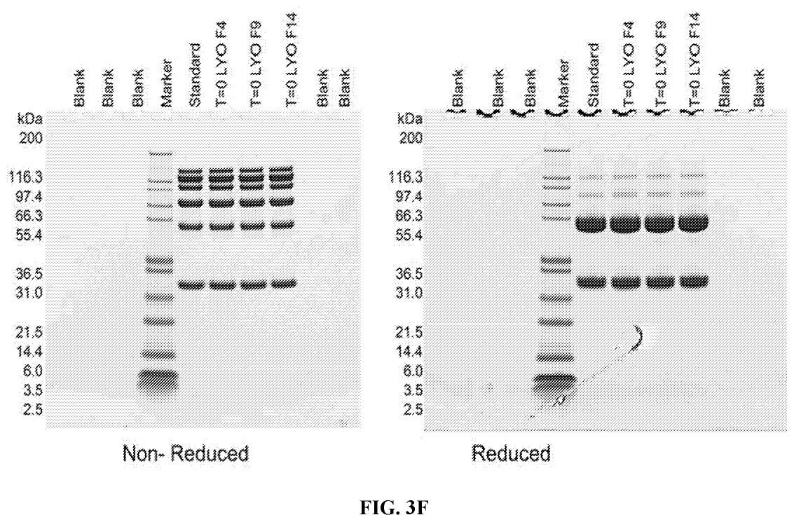

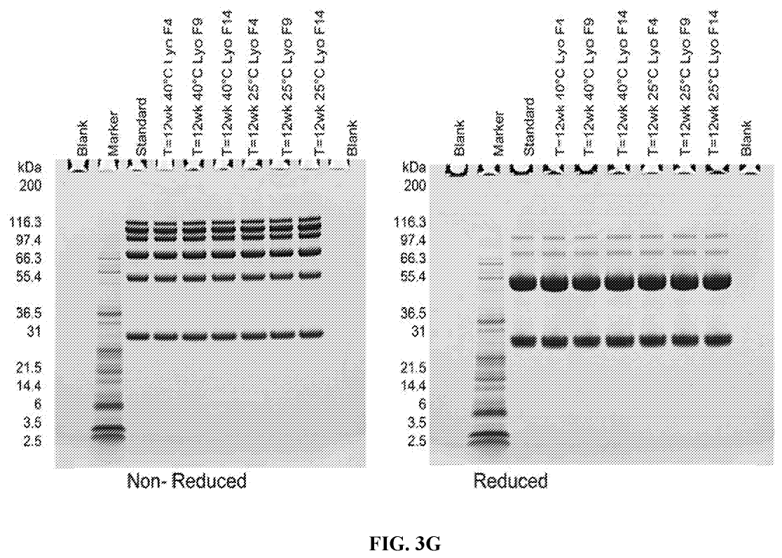

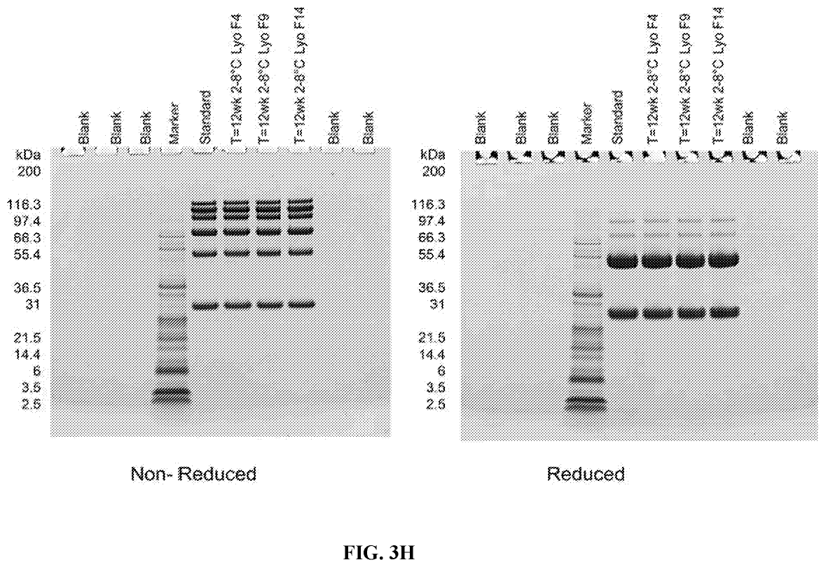

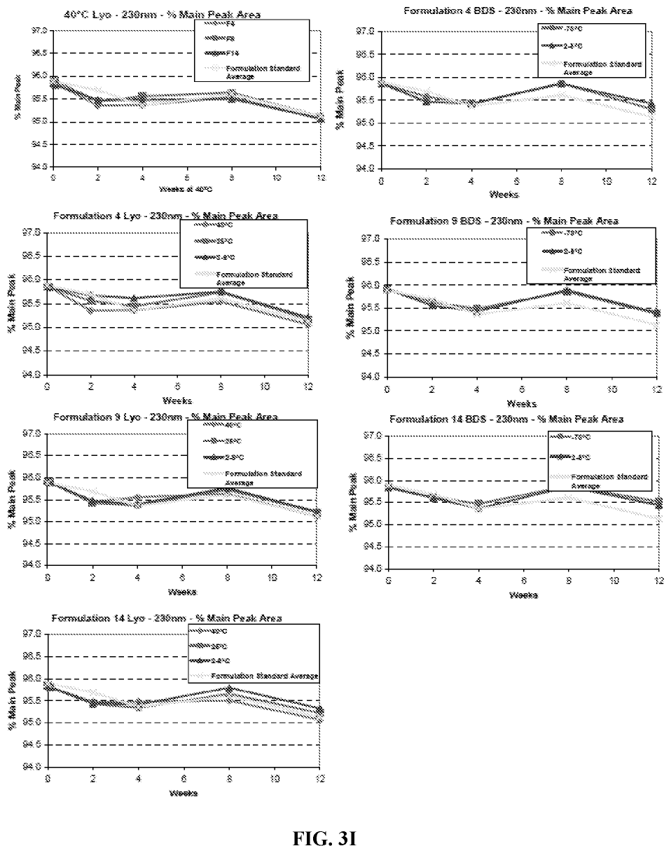

In some embodiments, the anti-191P4D12 antibody provided herein comprises heavy and light chain variable regions of an antibody designated Ha22-2(2,4)6.1 produced by a hybridoma deposited under the American Type Culture Collection (ATCC) Accession NO: PTA-11267 (See, FIG. 3), or heavy and light variable regions comprising amino acid sequences that are homologous to the amino acid sequences of the heavy and light chain variable regions of Ha22-2(2,4)6.1, and wherein the antibodies retain the desired functional properties of the anti-191P4D12 antibody provided herein. As the constant region of the antibody of the invention, any subclass of constant region can be chosen. In one embodiment, human IgG1 constant region as the heavy chain constant region and human Ig kappa constant region as the light chain constant region can be used.

In some embodiments, the anti-191P4D12 antibody provided herein comprises heavy and light chains of an antibody designated Ha22-2(2,4)6.1 produced by a hybridoma deposited under the American Type Culture Collection (ATCC) Accession NO: PTA-11267 (See, FIG. 3), or heavy and light chains comprising amino acid sequences that are homologous to the amino acid sequences of the heavy and light chains of Ha22-2(2,4)6.1, and wherein the antibodies retain the desired functional properties of the anti-191P4D12 antibody provided herein.

In some embodiments, the antibody or antigen binding fragment thereof provided herein comprises a heavy chain variable region and a light chain variable region, wherein: (a) the heavy chain variable region comprises an amino acid sequence that is at least 80% homologous to the heavy chain variable region amino acid sequence of the antibody produced by a hybridoma deposited under the American Type Culture Collection (ATCC) Accession NO: PTA-11267; and (b) the light chain variable region comprises an amino acid sequence that is at least 80% homologous to the light chain variable region amino acid sequence of the antibody produced by a hybridoma deposited under the American Type Culture Collection (ATCC) Accession NO: PTA-11267.

In some embodiments, the heavy chain variable region comprises an amino acid sequence that is at least 85% homologous to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous to the heavy chain variable region amino acid sequence of the antibody produced by a hybridoma deposited under the American Type Culture Collection (ATCC) Accession NO: PTA-11267. In other embodiments, the heavy chain variable region may be 85%, 86%, 87%, 88%, 89%, 90%, 91%, 92%, 93%, 94%, 95%, 96%, 97%, 98% or 99% homologous to the heavy chain variable region amino acid sequence of the antibody produced by a hybridoma deposited under the American Type Culture Collection (ATCC) Accession NO: PTA-11267.

In some embodiments, the light chain variable region comprises an amino acid sequence that is at least 85% homologous to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous to the light chain variable region amino acid sequence of the antibody produced by a hybridoma deposited under the American Type Culture Collection (ATCC) Accession NO: PTA-11267. In other embodiments, the light chain variable region may be 85%, 86%, 87%, 88%, 89%, 90%, 91%, 92%, 93%, 94%, 95%, 96%, 97%, 98% or 99% homologous to the light chain variable region amino acid sequence of the antibody produced by a hybridoma deposited under the American Type Culture Collection (ATCC) Accession NO: PTA-11267.

In other embodiments, the antibody or antigen binding fragment thereof provided herein comprises a heavy chain and a light chain, wherein: (a) the heavy chain comprises an amino acid sequence that is at least 80% homologous to the heavy chain amino acid sequence of the antibody produced by a hybridoma deposited under the American Type Culture Collection (ATCC) Accession NO: PTA-11267; and (b) the light chain comprises an amino acid sequence that is at least 80% homologous to the light chain amino acid sequence of the antibody produced by a hybridoma deposited under the American Type Culture Collection (ATCC) Accession NO: PTA-11267.

In some embodiments, the heavy chain comprises an amino acid sequence that is at least 85% homologous to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous to the heavy chain amino acid sequence of the antibody produced by a hybridoma deposited under the American Type Culture Collection (ATCC) Accession NO: PTA-11267. In other embodiments, the heavy chain may be 85%, 86%, 87%, 88%, 89%, 90%, 91%, 92%, 93%, 94%, 95%, 96%, 97%, 98% or 99% homologous to the heavy chain amino acid sequence of the antibody produced by a hybridoma deposited under the American Type Culture Collection (ATCC) Accession NO: PTA-11267.

In some embodiments, the light chain comprises an amino acid sequence that is at least 85% homologous to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous to the light chain amino acid sequence of the antibody produced by a hybridoma deposited under the American Type Culture Collection (ATCC) Accession NO: PTA-11267. In other embodiments, the light chain may be 85%, 86%, 87%, 88%, 89%, 90%, 91%, 92%, 93%, 94%, 95%, 96%, 97%, 98% or 99% homologous to the light chain amino acid sequence of the antibody produced by a hybridoma deposited under the American Type Culture Collection (ATCC) Accession NO: PTA-11267.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 236^{th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, VA 20108 on 18 Aug. 2010 and assigned Accession number PTA-11267.

### 5.3.2 Cytotoxic Agents (Drug Units)

In some embodiments, the ADC comprises an antibody or antigen binding fragment thereof conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug unit may be attached to the antibody through the N (amino) terminus or the C (carboxyl) terminus of the peptidic drug unit (WO 02/088172).

Exemplary auristatin embodiments include the N-terminus linked monomethylauristatin drug units DE and DF, disclosed in “Senter et al, Proceedings of the American Association for Cancer Research, Volume 45, Abstract Number 623, presented Mar. 28, 2004 and described in United States Patent Publication No. 2005/0238649, the disclosure of which is expressly incorporated by reference in its entirety.

In some embodiments, the auristatin is MMAE (wherein the wavy line indicates the covalent attachment to a linker of an antibody drug conjugate).

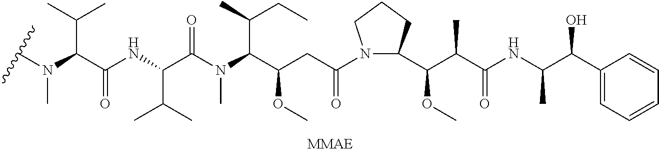 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody and p ranges from 1 to 12):

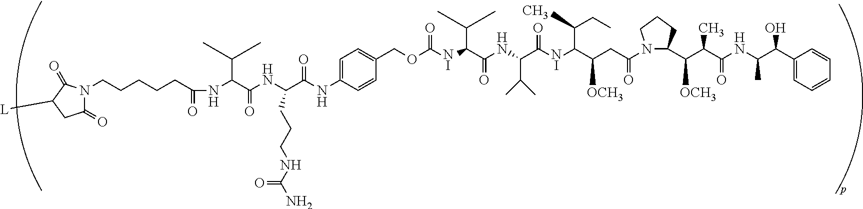

Typically, peptide-based drug units can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp 76-136, 1965, Academic Press) that is well-known in the field of peptide chemistry. The auristatin/dolastatin drug units may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol21(7):778-784.

### 5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in 191P4D12-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO:15)). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See U.S. Publication No. 2005/0238649 incorporated by reference herein in its entirety and for all purposes).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody drug conjugate, are cleaved when the antibody drug conjugate presents in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

In other non-mutually exclusive embodiments, the linker promotes cellular internalization. In certain embodiments, the linker promotes cellular internalization when conjugated to the therapeutic agent (i.e., in the milieu of the linker-therapeutic agent moiety of the antibody-drug conjugate compound as described herein). In yet other embodiments, the linker promotes cellular internalization when conjugated to both the auristatin compound and the anti-191P4D12 antibody or antigen binding fragment thereof.

A variety of exemplary linkers that can be used with the present compositions and methods are described in WO 2004-010957, U.S. Publication No. 2006/0074008, U.S. Publication No. 20050238649, and U.S. Publication No. 2006/0024317 (each of which is incorporated by reference herein in its entirety and for all purposes).

A “linker unit” (LU) is a bifunctional compound that can be used to link a drug unit and an antibody unit to form an antibody drug conjugate. In some embodiments, the linker unit has the formula:

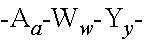

wherein: -A- is a stretcher unit,
a is 0 or 1,
each —W— is independently an amino acid unit,
w is an integer ranging from 0 to 12,
—Y— is a self-immolative spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0.

### 5.3.3.1 Stretcher Unit

The stretcher unit (A), when present, is capable of linking an antibody unit to an amino acid unit (—W—), if present, to a spacer unit (—Y—), if present; or to a drug unit (-D). Useful functional groups that can be present on an anti-191P4D12 antibody or an antigen binding fragment thereof (e.g. Ha22-2(2,4)6.1), either naturally or via chemical manipulation include, but are not limited to, sulfhydryl, amino, hydroxyl, the anomeric hydroxyl group of a carbohydrate, and carboxyl. Suitable functional groups are sulfhydryl and amino. In one example, sulfhydryl groups can be generated by reduction of the intramolecular disulfide bonds of an anti-191P4D12 antibody or an antigen binding fragment thereof. In another embodiment, sulfhydryl groups can be generated by reaction of an amino group of a lysine moiety of an anti-191P4D12 antibody or an antigen binding fragment with 2-iminothiolane (Traut's reagent) or other sulfhydryl generating reagents. In certain embodiments, the anti-191P4D12 antibody or antigen binding fragment thereof is a recombinant antibody and is engineered to carry one or more lysines. In certain other embodiments, the recombinant anti-191P4D12 antibody is engineered to carry additional sulfhydryl groups, e.g., additional cysteines.

In one embodiment, the stretcher unit forms a bond with a sulfur atom of the antibody unit. The sulfur atom can be derived from a sulfhydryl group of an antibody. Representative stretcher units of this embodiment are depicted within the square brackets of Formulas IIIa and IIIb below, wherein L-, —W—, —Y—, -D, w and y are as defined above, and R″ is selected from —C₁-C₁₀ alkylene-, —C₁-C₁₀ alkenylene-, —C₁-C₁₀ alkynylene-, carbocyclo-, —O—(C₁-C₈ alkylene)-, O—(C₁-C₈ alkenylene)-, —O—(C₁-C₈ alkynylene)-, -arylene-, —C₁-C₁₀ alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene-(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, -heterocyclo-, —C₁-C₁₀ alkylene-(heterocyclo)-, —C₂-C₁₀ alkenylene-(heterocyclo)-, —C₂-C₁₀ alkynylene-(heterocyclo)-, -(heterocyclo)-C₁-C₁₀ alkylene-, -(heterocyclo)-C₂-C₁₀ alkenylene-, -(heterocyclo)-C₁-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynylene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynylene, aryl, carbocycle, carbocyclo, heterocyclo, and arylene radicals, whether alone or as part of another group, are unsubstituted.

In some embodiments, R¹⁷ is selected from —C₁-C₁₀ alkylene-, -carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(carbocyclo)-, -(carbocyclo)-C₁-C₁₀ alkylene-, —C₃-C₈ heterocyclo-, —C₁-C₁₀ alkylene-(heterocyclo)-, -(heterocyclo)-C₁-C₁₀ alkylene-, —(CH₂CH₂O)ᵣ—, and —(CH₂CH₂O)ᵣ—CH₂—; and r is an integer ranging from 1-10, wherein said alkylene groups are unsubstituted and the remainder of the groups are optionally substituted.

It is to be understood from all the exemplary embodiments that even where not denoted expressly, 1 to 20 drug units can be linked to an antibody unit (p=1-20).

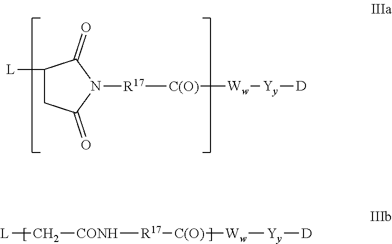

An illustrative stretcher unit is that of Formula IIIa wherein R⁷ is —(CH₂)₅—:

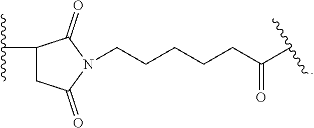 [SMILES: *C(=O)CCCCCN1C(=O)CC(*)C1=O]

Another illustrative stretcher unit is that of Formula IIIa wherein R¹⁷ is —(CH₂CH₂O)ᵣ—CH₂—; and r is 2:

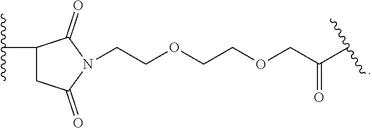 [SMILES: *C(=O)COCCOCCN1C(=O)CC(*)C1=O]

An illustrative Stretcher unit is that of Formula IIIa wherein R¹⁷ is arylene- or arylene-C₁-C₁₀ alkylene-. In some embodiments, the aryl group is an unsubstituted phenyl group.

Still another illustrative stretcher unit is that of Formula IIIb wherein R¹⁷ is —(CH₂)₅—:

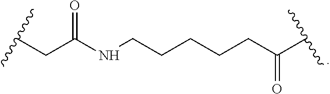 [SMILES: *CC(=O)NCCCCCC(*)=O]

In certain embodiments, the stretcher unit is linked to the antibody unit via a disulfide bond between a sulfur atom of the antibody unit and a sulfur atom of the stretcher unit. A representative stretcher unit of this embodiment is depicted within the square brackets of Formula IV, wherein R¹⁷, L-, —W—, —Y—, -D, w and y are as defined above.

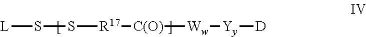

It should be noted that throughout this application, the S moiety in the formula below refers to a sulfur atom of the antibody unit, unless otherwise indicated by context.

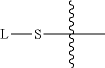

In certain of the structural descriptions of sulfur linked ADC herein the antibody is represented as “L”. It could also be indicated as “Ab-S”. The inclusion of “S” merely indicated the sulfur-linkage feature, and does not indicate that a particular sulfur atom bears multiple linker-drug moieties. The left parentheses of the structures using the “Ab-S” description may also be placed to the left of the sulfur atom, between Ab and S, which would be an equivalent description of the ADC of the invention described throughout herein.

In yet other embodiments, the stretcher contains a reactive site that can form a bond with a primary or secondary amino group of an antibody unit. Examples of these reactive sites include, but are not limited to, activated esters such as succinimide esters, 4 nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas Va and Vb, wherein —R¹⁷—, L-, —W—, —Y—, -D, w and y are as defined above;

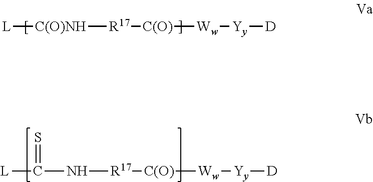

In some embodiments, the stretcher contains a reactive site that is reactive to a modified carbohydrate's (—CHO) group that can be present on an antibody unit. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko et al., 1991, Bioconjugate Chem. 2:133-41. Representative stretcher units of this embodiment are depicted within the square brackets of Formulas VIa, VIb, and VIc, wherein —R¹⁷—, L-, —W—, —Y—, -D, w and y are as defined as above.

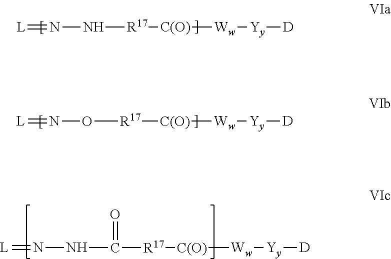

### 5.3.3.2 Amino Acid Unit

The amino acid unit (—W—), when present, links the stretcher unit to the spacer unit if the spacer unit is present, links the stretcher unit to the drug unit if the spacer unit is absent, and links the antibody unit to the drug unit if the stretcher unit and spacer unit are absent.

W_{w}— can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each —W— unit independently has the formula denoted below in the square brackets, and w is an integer ranging from 0 to 12:

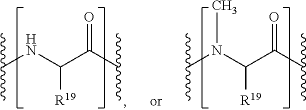

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

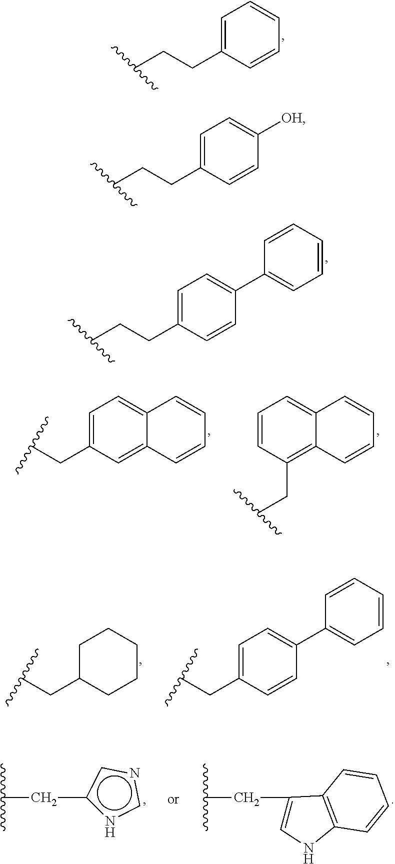

In some embodiments, the amino acid unit can be enzymatically cleaved by one or more enzymes, including a cancer or tumor-associated protease, to liberate the drug unit (-D), which in one embodiment is protonated in vivo upon release to provide a drug (D).

In certain embodiments, the amino acid unit comprises natural amino acids. In other embodiments, the amino acid unit comprises non-natural amino acids. Illustrative Ww units are represented by Formulas VII-IX below:

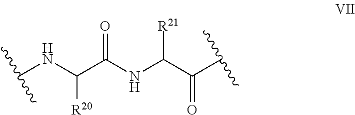

wherein R²⁰ and R²¹ are as follows:

| R²⁰ | R²¹ |
|---|---|
| Benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 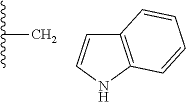 | (CH₂)₃NHCONH₂; |
| benzyl | methyl; |
| benzyl | (CH₂)₃NHC(═NH)NH₂; |

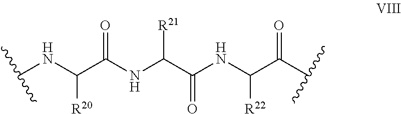

wherein R²⁰, R²¹ and R²² are as follows:

| R²⁰ | R²¹ | R²² |
|---|---|---|
| benzyl | benzyl | (CH₂)₄NH₂; |
| isopropyl | benzyl | (CH₂)₄NH₂; and |
| H | benzyl | (CH₂)₄NH₂; |

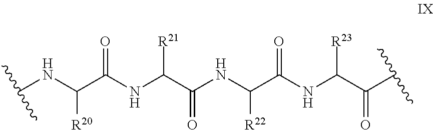

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl. |

Exemplary amino acid units include, but are not limited to, units of Formula VII above where: R²⁰ is benzyl and R²¹ is —(CH₂)₄NH₂; R²⁰ is isopropyl and R²¹ is —(CH₂)₄NH₂; or R²⁰ is isopropyl and R²¹ is —(CH₂)₃NHCONH₂.

Another exemplary amino acid unit is a unit of Formula VIII wherein R²⁰ is benzyl, R²¹ is benzyl, and R²² is —(CH₂)₄NH₂.

Useful —W_{w}— units can be designed and optimized in their selectivity for enzymatic cleavage by a particular enzyme, for example, a tumor-associated protease. In one embodiment, a —W_{w}— unit is that whose cleavage is catalyzed by cathepsin B, C and D, or a plasmin protease.

In one embodiment, —W_{w}— is a dipeptide, tripeptide, tetrapeptide or pentapeptide. When R¹⁹, R²⁰, R²¹, R²² or R²³ is other than hydrogen, the carbon atom to which R¹⁹, R²⁰, R²¹ R²² or R²³ is attached is chiral.

Each carbon atom to which R¹⁹, R²⁰, R²¹, R²² or R²³ is attached is independently in the (S) or (R) configuration.

In one specific embodiment, the amino acid unit is valine-citrulline (vc or Val-Cit). In another specific embodiment, the amino acid unit is phenylalanine-lysine (i.e., fk). In yet another specific embodiment, the amino acid unit is N-methylvaline-citrulline. In yet another specific embodiment, the amino acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

### 5.3.3.3 Spacer Unit

The spacer unit (—Y—), when present, links an amino acid unit to the drug unit when an amino acid unit is present. Alternately, the spacer unit links the stretcher unit to the drug unit when the amino acid unit is absent. The spacer unit also links the drug unit to the antibody unit when both the amino acid unit and stretcher unit are absent.

Spacer units are of two general types: non self-immolative or self-immolative. A non self-immolative spacer unit is one in which part or all of the spacer unit remains bound to the drug unit after cleavage, particularly enzymatic, of an amino acid unit from the antibody drug conjugate. Examples of a non self-immolative spacer unit include, but are not limited to a (glycine-glycine) spacer unit and a glycine spacer unit (both depicted in Scheme 1) (infra). When a conjugate containing a glycine-glycine spacer unit or a glycine Spacer unit undergoes enzymatic cleavage via an enzyme (e.g., a tumor-cell associated-protease, a cancer-cell-associated protease or a lymphocyte-associated protease), a glycine-glycine-drug unit or a glycine-drug unit is cleaved from L-Aa-Ww-. In one embodiment, an independent hydrolysis reaction takes place within the target cell, cleaving the glycine-drug unit bond and liberating the drug.

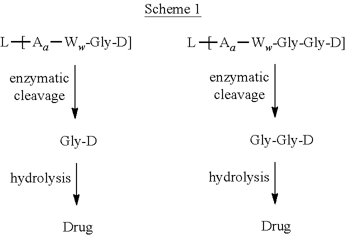

In some embodiments, a non self-immolative spacer unit (—Y—) is -Gly-. In some embodiments, a non self-immolative spacer unit (—Y—) is -Gly-Gly-.

In one embodiment, the spacer unit is absent (—Y_{y}— where y=0).

Alternatively, an antibody drug conjugate containing a self-immolative spacer unit can release -D. As used herein, the term “self-immolative spacer” refers to a bifunctional chemical moiety that is capable of covalently linking together two spaced chemical moieties into a stable tripartite molecule. It will spontaneously separate from the second chemical moiety if its bond to the first moiety is cleaved.

In some embodiments, —Y_{y}— is a p-aminobenzyl alcohol (PAB) unit (see Schemes 2 and 3) whose phenylene portion is substituted with Qₘ wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

In some embodiments, —Y— is a PAB group that is linked to —W_{w}— via the amino nitrogen atom of the PAB group, and connected directly to -D via a carbonate, carbamate or ether group. Without being bound by any particular theory or mechanism, Scheme 2 depicts a possible mechanism of Drug release of a PAB group which is attached directly to -D via a carbamate or carbonate group as described by Toki et al., 2002, J. Org. Chem. 67:1866-1872.

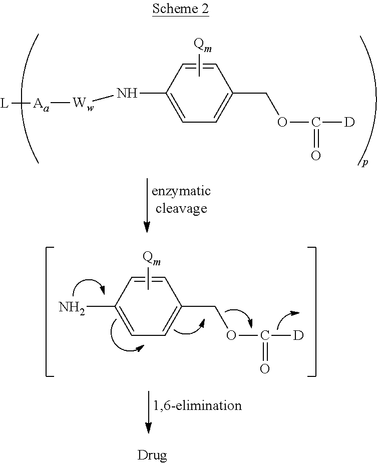

In Scheme 2, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Without being bound by any particular theory or mechanism, Scheme 3 depicts a possible mechanism of drug release of a PAB group which is attached directly to -D via an ether or amine linkage, wherein D includes the oxygen or nitrogen group that is part of the drug unit.

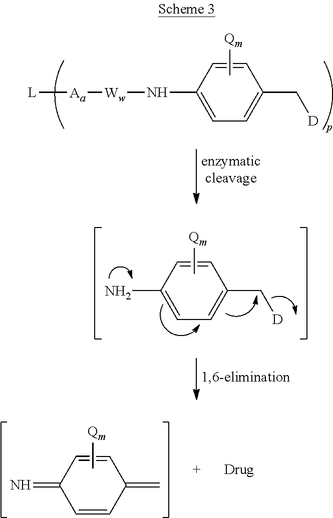

In Scheme 3, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Other examples of self-immolative spacers include, but are not limited to, aromatic compounds that are electronically similar to the PAB group such as 2-aminoimidazol-5-methanol derivatives (Hay et al., 1999, Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., 1995, Chemistry Biology 2:223), appropriately substituted bicyclo[2.2.1] and bicyclo[2.2.2] ring systems (Storm et al., 1972, J. Amer. Chem. Soc. 94:5815) and 2-aminophenylpropionic acid amides (Amsberry et al., 1990, J. Org. Chem. 55:5867). Elimination of amine-containing drugs that are substituted at the α-position of glycine (Kingsbury et al., 1984, J. Med. Chem. 27:1447) are also examples of self-immolative spacers.

In one embodiment, the spacer unit is a branched bis(hydroxymethyl)-styrene (BHMS) unit as depicted in Scheme 4, which can be used to incorporate and release multiple drugs.

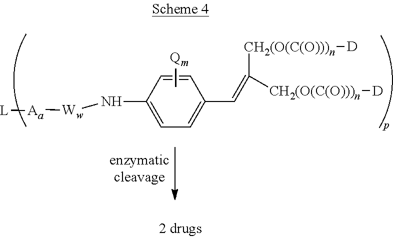

In Scheme 4, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; n is 0 or 1; and p ranges ranging from 1 to about 20. The alkyl, alkenyl and alkynyl groups, whether alone or as part of another group, can be optionally substituted.

In some embodiments, the -D units are the same. In yet another embodiment, the -D moieties are different.

In one aspect, spacer units (—Y_{y}—) are represented by Formulas X-XII:

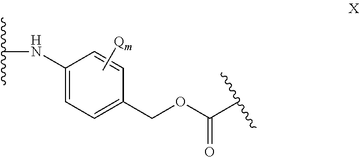

wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

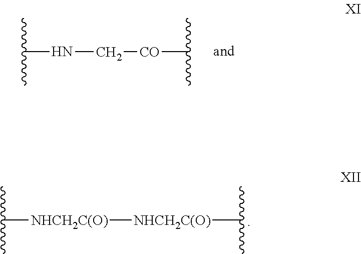

Embodiments of the Formula I and II comprising antibody-drug conjugate compounds can include:

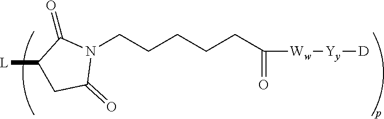

wherein w and y are each 0, 1 or 2, and,

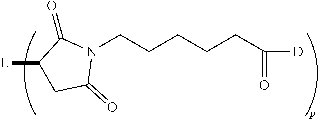

wherein w and y are each 0,

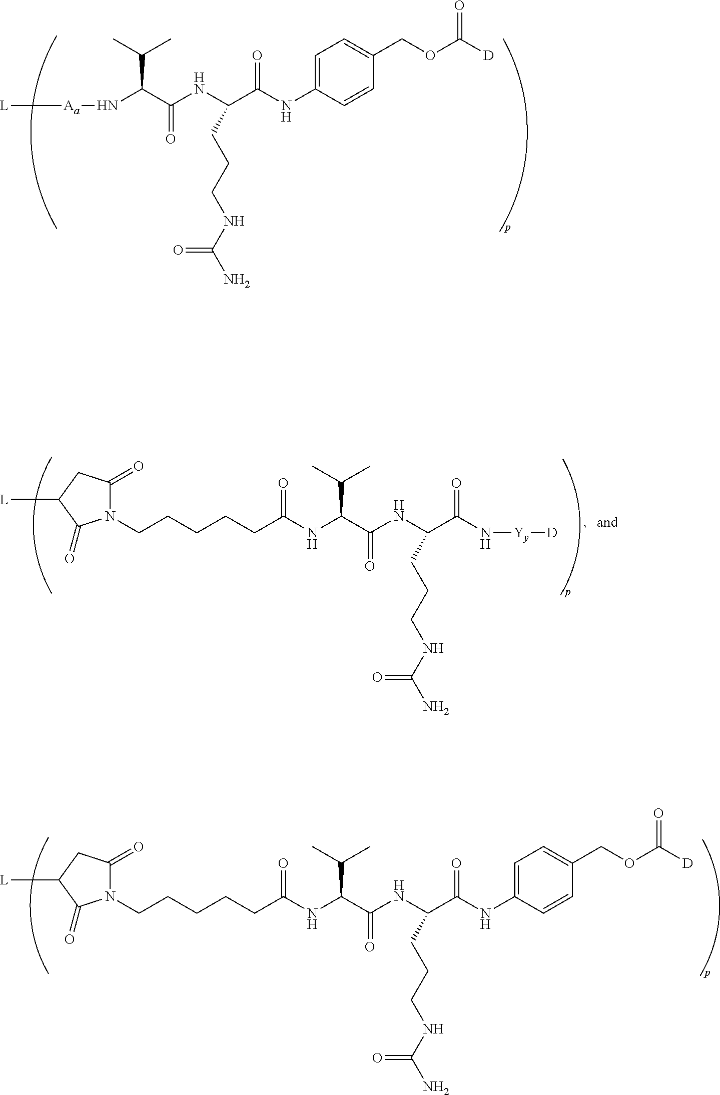

### 5.3.3.4 Drug Loading

Drug loading is represented by p and is the average number of drug units per antibody in a molecule. Drug loading may range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions may be characterized by conventional means such as mass spectroscopy and, ELISA assay. The quantitative distribution of ADC in terms of p may also be determined. In some instances, separation, purification, and characterization of homogeneous ADC where p is a certain value from ADC with other drug loadings may be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, fewer than the theoretical maximum of drug units are conjugated to an antibody during a conjugation reaction. An antibody may contain, for example, lysine residues that do not react with the drug-linker intermediate or linker reagent. Generally, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, March 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

### 5.3.3 Preparation of the Antibody Drug Conjugates

The generation of antibody drug conjugates provided herein can be accomplished by any technique known to the skilled artisan. Briefly, the antibody drug conjugates comprise an anti-191P4D12 antibody or antigen binding fragment thereof as the antibody unit, a drug, and optionally a linker that joins the drug and the binding agent. In some embodiments, the antibody is anti-191P4D12 antibody comprising the CDR regions of an antibody designated Ha22-2(2,4)6.1 described above. In a specific embodiment, the antibody is anti-191P4D12 antibody comprising heavy and light chain variable regions of an antibody designated Ha22-2(2,4)6.1 described above. In a specific embodiment, the antibody is anti-191P4D12 antibody comprising heavy and light chain of an antibody designated Ha22-2(2,4)6.1 described above.

A number of different reactions are available for covalent attachment of drugs and/or linkers to binding agents. This is often accomplished by reaction of the amino acid residues of the binding agent, e.g., antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates can also be used as coupling agents for covalently attaching drugs to binding agents. Other techniques are known to the skilled artisan and within the scope of the present invention.

In certain embodiments, an intermediate, which is the precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the anti-191P4D12 antibody under appropriate conditions.

Each of the particular units of the antibody drug conjugates is described in more detail herein. The synthesis and structure of exemplary linker units, stretcher units, amino acid units, self-immolative spacer unit, and drug units are also described in U.S. Patent Application Publication Nos. 2003-0083263, 2005-0238649 and 2005-0009751, each of which is incorporated herein by reference in its entirety and for all purposes.

An exemplary method for generating the antibody drug conjugates provided herein is described briefly below.

The Ha22-2(2,4)6.1 antibody is conjugated to an auristatin derivative MMAE using a vc (Val-Cit) linker described herein to create the antibody drug conjugate (ADC) (designated as AGS-22M6E) using the following protocols. The conjugation of the vc (Val-Cit) linker to the MMAE (Seattle Genetics, Inc., Seattle, WA) was completed using the general method set forth in Scheme 5 below to create the cytotoxic vcMMAE (see, U.S. Pat. No. 7,659,241).

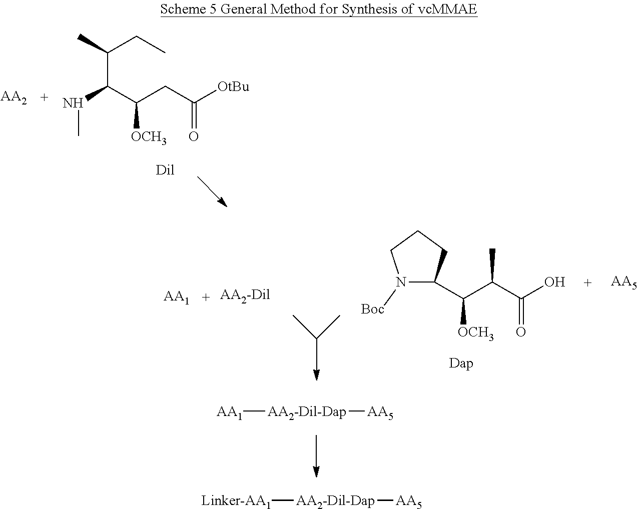

Where: AA1=Amino Acid 1
   AA2=Amino Acid 2
   AA5=Amino Acid 5
   DIL=Dolaisoleuine
   DAP=Dolaproine
   Linker=Val-Cit (vc)

Next, the antibody drug conjugate AGS-22M6E was made using the following protocols.

Briefly, a 15 mg/mL solution of the Ha22-2(2,4)6.1 antibody in 10 mM acetate at pH 5.0, 1% sorbitol, 3% L-agrinine is added with a 20% volume of 0.1 M TrisCl at pH 8.4, 25 mM EDTA and 750 mM NaCl to adjust the pH of the solution to 7.5, 5 mM EDTA and 150 mM sodium chloride. The antibody is then partially reduced by adding 2.3 molar equivalents of TCEP (relative to moles of MAb) and then stirred at 37° C. for 2 hours. The partially reduced antibody solution is then cooled to 5° C. and 4.4 molar equivalents of vcMMAE (relative to moles of antibody) are added as a 6% (v/v) solution of DMSO. The mixture is stirred for 60 minutes at 5° C., then for 15 additional minutes following the addition of 1 molar equivalents of N-acetylcysteine relative to vcMMAE. Excess quenched vcMMAE and other reaction components are removed by ultrafiltration/diafiltration of the antibody drug conjugate (ADC) with 10 volumes of 20 mM histidine, pH 6.0.

The resulting antibody drug conjugate AGS-22M6E has the following formula:

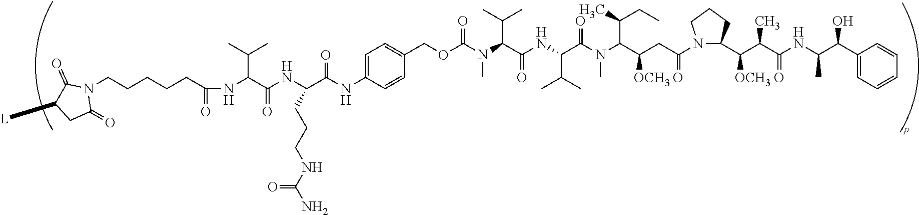

wherein L is Ha22-2(2,4)6.1 and p is from 1 to 20.

### 5.4 Methods of Using the Pharmaceutical Compositions

In one aspect, provided herein is a method of preventing or treating a disease or disorder in a subject comprising administering to the subject an effective amount of the pharmaceutical composition provided herein. In some embodiments, the subject is a human subject.

In some embodiments, the disease or disorder is cancer. In some embodiments, the cancer has tumor cells expressing 191P4D12. In some embodiments, the cancer is a solid tumor. In some embodiments, the cancer is colon cancer, pancreatic cancer, ovarian cancer, lung cancer, bladder cancer, breast cancer, esophageal cancer, head cancer, or neck cancer. In some embodiments, the cancer is colon cancer. In some embodiments, the cancer is pancreatic cancer. In some embodiments, the cancer is ovarian cancer. In some embodiments, the cancer is lung cancer. In some embodiments, the lung cancer is non-small cell lung cancer. In some embodiments, the cancer is bladder cancer. In some embodiments, the cancer is advanced bladder cancer. In some embodiments, the cancer is metastatic bladder cancer. In some embodiments, the cancer is urothelial cancer. In some embodiments, the cancer is advanced urothelial cancer. In some embodiments, the cancer is breast cancer. In some embodiments, the cancer is esophageal cancer. In some embodiments, the cancer is head cancer. In some embodiments, the cancer is neck cancer. In some embodiments, the cancer is advanced or metastatic cancer.

In some embodiments, treatment with the pharmaceutical composition provided herein is indicated for subjects who have received one or more rounds of chemotherapy. Alternatively, the pharmaceutical composition provided herein is combined with a chemotherapeutic or radiation regimen for subjects who have not received chemotherapeutic treatment. Additionally, in some embodiments, use of the pharmaceutical composition provided herein can enable the use of reduced dosages of concomitant chemotherapy, particularly for subjects who do not tolerate the toxicity of the chemotherapeutic agent very well. In some embodiments, the pharmaceutical composition disclosed herein is administered to patients with metastatic urothelial cancer who have shown disease progression or relapse during or after treatment with an immune checkpoint inhibitor.

Methods of administering the pharmaceutical composition provided herein include, but are not limited to, parenteral administration (e.g., intradermal, intramuscular, intraperitoneal, intravenous and subcutaneous), epidural, and mucosal (e.g., intranasal and oral routes). In a specific embodiment, the pharmaceutical composition provided herein is administered intranasally, intramuscularly, intravenously, or subcutaneously. The pharmaceutical composition provided herein may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer the pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering the pharmaceutical composition provided herein, care must be taken to use materials to which the antibody drug conjugate provided herein does not absorb.

In another embodiment, the pharmaceutical composition provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, the pharmaceutical composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibody drug conjugate provided herein) or a pharmaceutical composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising the antibody drug conjugate or pharmaceutical composition provided herein. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

The amount of the pharmaceutical composition provided herein that will be effective in the prevention and/or treatment of a cancer can be determined by standard clinical techniques. In addition, in vitro assays may optionally be employed to help identify optimal dosage ranges. The precise dose to be employed will also depend on the route of administration, and the seriousness of a disease or disorder, and should be decided according to the judgment of the practitioner and each subject's circumstances. Effective doses may be extrapolated from dose-response curves derived from in vitro or animal model test systems.

In certain embodiments, therapeutic methods provided herein contemplate the administration of a single ADC as well as combinations, or cocktails, of different ADCs comprising different anti-191P4D12 antibodies or different drug units. In some embodiments, such methods have certain advantages because, e.g., they contain ADCs that target different epitopes, exploit different effector mechanisms or combine directly cytotoxic antibodies with antibodies that rely on immune effector functionality. Such methods can exhibit synergistic therapeutic effects. In addition, the pharmaceutical composition provided herein can be administered concomitantly with other therapeutic modalities, including but not limited to various chemotherapeutic and biologic agents, androgen-blockers, immune modulators (e.g., IL-2, GM-CSF), surgery or radiation.

In one embodiment, there is synergy when tumors, including human tumors, are treated with the pharmaceutical composition provided herein in conjunction with chemotherapeutic agents or radiation or combinations thereof.

The method for inhibiting growth of tumor cells using the pharmaceutical composition provided herein and a combination of chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient.

The administration of chemotherapeutic agents can be accomplished in a variety of ways including systemically by the parenteral and enteral routes. In one embodiment, the chemotherapeutic agent is administered separately. Particular examples of chemotherapeutic agents or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, gemcitabine, chlorambucil, taxol and combinations thereof.

The source of radiation, used in combination with the pharmaceutical composition provided herein, can be either external or internal to the patient being treated. When the source is external to the patient, the therapy is known as external beam radiation therapy (EBRT). When the source of radiation is internal to the patient, the treatment is called brachytherapy (BT).

The above described therapeutic regimens may be further combined with additional cancer treating agents and/or regimes, for example additional chemotherapy, cancer vaccines, signal transduction inhibitors, agents useful in treating abnormal cell growth or cancer, antibodies (e.g. Anti-CTLA-4 antibodies as described in WO/2005/092380 (Pfizer)) or other ligands that inhibit tumor growth by binding to IGF-1R, and cytokines.

When the mammal is subjected to additional chemotherapy, chemotherapeutic agents described above may be used. Additionally, growth factor inhibitors, biological response modifiers, anti-hormonal therapy, selective estrogen receptor modulators (SERMs), angiogenesis inhibitors, and anti-androgens may be used. For example, anti-hormones, for example anti-estrogens such as Nolvadex (tamoxifen) or, anti-androgens such as Casodex (4′-cyano-3-(4-fluorophenylsulphonyl)-2-hydroxy-2-methyl-3-′-(trifluoromethyl)propionanilide) may be used.

In some embodiments, the pharmaceutical provided herein in used in combination with a second therapeutic agent, e.g., for treating a cancer.

In some embodiments, the second therapeutic agent is an immune checkpoint inhibitor. As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with one or more second active agents as described herein where appropriate for treating diseases described herein and understood in the art.

In some embodiments, the checkpoint inhibitor is administered prior to the administration of the present pharmaceutical composition. In other embodiments, the checkpoint inhibitor is administered simultaneously (e.g., in the same dosing period) with the pharmaceutical composition provided herein. In yet other embodiments, the checkpoint inhibitor is administered after the administration of the pharmaceutical composition provided herein.

In some embodiments, the amount of the checkpoint inhibitor can be determined by standard clinical techniques.

A dosage of the checkpoint inhibitor results in a serum titer of from about 0.1 g/ml to about 450 g/ml, and in some embodiments at least 0.1 g/ml, at least 0.2 g/ml, at least 0.4 g/ml, at least 0.5 g/ml, at least 0.6 g/ml, at least 0.8 g/ml, at least 1 g/ml, at least 1.5 g/ml, such as at least 2 g/ml, at least 5 g/ml, at least 10 g/ml, at least 15 g/ml, at least 20 g/ml, at least 25 g/ml, at least 30 g/ml, at least 35 g/ml, at least 40 g/ml, at least 50 g/ml, at least 75 g/ml, at least 100 g/ml, at least 125 g/ml, at least 150 g/ml, at least 200 g/ml, at least 250 g/ml, at least 300 g/ml, at least 350 g/ml, at least 400 g/ml, or at least 450 g/ml can be administered to a human for the prevention and/or treatment of a cancer. It is to be understood that the precise dose of the checkpoint inhibitor to be employed will also depend on the route of administration, and the seriousness of a cancer in a subject, and should be decided according to the judgment of the practitioner and each patient's circumstances.

In some embodiments, the dosage of the checkpoint inhibitor (e.g., a PD-1 inhibitor or a PD-L1 inhibitor) administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight. In some embodiments, dosage administered to a patient is about 9.0 mg/kg of the subject's body weight. In some embodiments, dosage administered to a patient is about 10.0 mg/kg of the subject's body weight. In some embodiments, dosage administered to a patient is about 15.0 mg/kg of the subject's body weight. In some embodiments, dosage administered to a patient is about 20.0 mg/kg of the subject's body weight.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, or at least 3 mg, such as at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, or at least 1 mg/ml, and such as at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

Accordingly, a dosage of an antibody drug conjugate in the pharmaceutical composition that results in a serum titer of from about 0.1 g/ml to about 450 g/ml, and in some embodiments at least 0.1 g/ml, at least 0.2 g/ml, at least 0.4 g/ml, at least 0.5 g/ml, at least 0.6 g/ml, at least 0.8 g/ml, at least 1 g/ml, at least 1.5 g/ml, such as at least 2 g/ml, at least g/ml, at least 10 g/ml, at least 15 g/ml, at least 20 g/ml, at least 25 g/ml, at least 30 g/ml, at least 35 g/ml, at least 40 g/ml, at least 50 g/ml, at least 75 g/ml, at least 100 g/ml, at least 125 g/ml, at least 150 g/ml, at least 200 g/ml, at least 250 g/ml, at least 300 g/ml, at least 350 g/ml, at least 400 g/ml, or at least 450 g/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses may be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by >10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses may be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous, or a combination thereof, but other routes described herein are also acceptable. Each dose may or may not be administered by an identical route of administration. In some embodiments, an antibody drug conjugate formulated in the pharmaceutical composition provided herein may be administered via multiple routes of administration simultaneously or subsequently to other doses of one or more additional therapeutic agents.

In some more specific embodiments, the antibody drug conjugate formulated in the pharmaceutical composition provided herein is administered at a dose of about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 1 mg/kg, 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion one or more times in each three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In other embodiments, the immune checkpoint inhibitor is atezolizumab, and wherein atezolizumab is administered at amount of about 1200 mg over about 60 minutes or 30 minutes. In some embodiments, the antibody drug conjugate is administered to patients with urothelial cancer who have shown disease progression or relapse during or after treatment with an immune checkpoint inhibitor. In some embodiments, the antibody drug conjugate is administered to patients with metastatic urothelial cancer who have shown disease progression or relapse during or after treatment with an immune checkpoint inhibitor.

In other more specific embodiments, the antibody drug conjugate formulated in the pharmaceutical composition provided herein is administered at a dose of about 1 mg/kg, 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered by an intravenous (IV) injection or infusion over about 30 minutes on Days 1, 8 and 15 of every four-week cycle. In some embodiments, the method further comprises administering an immune checkpoint inhibitor by an intravenous (IV) injection or infusion one or more times in each four-week cycle. In some embodiments, the immune checkpoint inhibitor is pembrolizumab. In other embodiments, the immune checkpoint inhibitor is atezolizumab. In some embodiments, the antibody drug conjugate is administered to patients with urothelial cancer who have shown disease progression or relapse during or after treatment with an immune checkpoint inhibitor. In some embodiments, the antibody drug conjugate is administered to patients with metastatic urothelial cancer who have shown disease progression or relapse during or after treatment with an immune checkpoint inhibitor.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 6.1 Example 1—pH and Buffer Screen

AGS-22M6E was formulated at 10 mg/mL in fourteen candidate buffers (all at 20 mM, as detailed in Table 1 below). Formulations using 20 mM sodium citrate buffer titrated to pH 5.2 and 5.7 with citric acid, and 20 mM Histidine buffer titrated to pH 5.5, 6.0 and 6.5 with HCl were evaluated. In addition, three different anions were evaluated in the histidine buffer systems-chloride, phosphate and succinate. The liquid formulations were subjected to 40° C. storage temperature condition for 2 weeks, room temperature (RT) agitation for 24 hours, and freeze-thaw cycles (freezing at −70° C. and thawing at 20° C.-25° C. for 1, 3 and 10 cycles).

TABLE 1

| | | | Trehalose | | Tween 20 |
|---|---|---|---|---|---|
| Formulation | | | Dihydrate | Sucrose | Conc. |
| # | Buffer | pH | (%) | (%) | (w/v %) |
| F1 | 20 mM sodium | 5.2 | 5.5 | 0 | 0.02 |
| F2 | citrate/citric acid | 5.7 | | | |
| F3 | 20 mM histidine/ | 5.5 | | | |
| F4 | HCl | 6 | | | |
| F5 | | 6.5 | | | |
| F6 | 20 mM sodium | 5.2 | 0 | 5 | |
| F7 | citrate/citric acid | 5.7 | | | |
| F8 | 20 mM histidine/ | 5.5 | | | |
| F9 | HCl | 6 | | | |
| F10 | | 6.5 | | | |
| F11 | 20 mM histidine/ | 5.5 | 5.5 | 0 | |
| F12 | Phosphoric acid | 6 | | | |
| F13 | 20 mM histidine/ | 5.5 | | | |
| F14 | Succinic acid | 6 | | | |

Note:
5% sucrose (m. wt. 342) = 146 mM; 5.5% trehalose dihydrate (m. wt. 378) = 146 mM.

Formulation preparation and study design are described in more detail below.

### Protein Product Used for Formulation Study

Four tubes each containing about 50 ml of AGS-22M6E (Lot #AGS22M6-VCE-02), totaling about 2.5 grams were received frozen. AGS-22M6E was at 12.5 mg/mL in 20 mM histidine pH 6.0 buffer containing 5% sucrose and 0.02% polysorbate-20. The material was stored at −70° C. until used.

### Preparation of Formulation Buffers

Stock solutions including citric acid (0.1 M), sodium citrate (0.1M) and L-histidine (0.2 M), succinic acid (0.25M), trehalose Dihydrate (40%), sucrose (40%), hydrochloric Acid (2M) and phosphoric Acid (2M) were prepared according to Table 2 below:

TABLE 2

| | | Desired | | Mass |
|---|---|---|---|---|
| | MW | Concentration | Volume | Required |
| Stocks | (g/mol) | (mol/L) | (L) | (g) |
| Citric Acid | 210.14 | 0.1 | 1.0 | 21.01 |
| Monohydrate | | | | |
| Sodium Citrate | 294.1 | 0.1 | 2.0 | 58.82 |
| Dihydrate | | | | |
| L-Histidine | 155.15 | 0.2 | 1.5 | 46.55 |
| Trehalose | 378.33 | 40% | 1.5 | 600.00 |
| Dihydrate | | | | |
| Sucrose | 342.30 | 40% | 1.0 | 400.00 |
| Succinic Acid | 118.09 | 0.25 | 0.8 | 23.62 |

| | Initial | Final | Final | To measure out | |
|---|---|---|---|---|---|
| | Conc | Conc | Volume | Concentrated | Water |
| Stocks | (M) | (M) | (mL) | Acid (mL) | (mL) |
| Hydrochloric | 12.1 | 2.0 | 500 | 82.6 | 417.4 |
| Acid | | | | | |
| Phosphoric | 14.8 | 2.0 | 500 | 67.6 | 432.4 |
| Acid | | | | | |

Reagents were weighed out according to the table above. Appropriate volume of Milli-Q water was added to dissolve the reagents. The solutions were filtered through 0.22 um filters.

### Preparation of Formulation Buffers for Dialysis

1.0 L of each formulation was prepared for dialysis and placebo vialing according to Table 3 below:

TABLE 3

| | | 0.1M Citric | 0.1M Sodium | | 40% | | | | |
|---|---|---|---|---|---|---|---|---|---|
| | | Acid | Citrate | 0.2M | Trehalose | 40% | | pH | Total |
| | | Monohydrate | dihydrate | L-Histidine | Dihydrate | Sucrose | Water | Adjustment | volume |
| Formulation # | pH | (mL) | (mL) | (mL) | (mL) | (mL) | (mL) | with | (mL) |
| F1 | 5.2 | 61 | 139 | | 137.5 | | 662.5 | | 1000 |
| F2 | 5.7 | 37 | 163 | | 137.5 | | 662.5 | | 1000 |
| F3 | 5.5 | | | 100 | 137.5 | | 762.5 | 2M HCl | 1000 |
| F4 | 6.0 | | | 100 | 137.5 | | 762.5 | 2M HCl | 1000 |
| F5 | 6.5 | | | 100 | 137.5 | | 762.5 | 2M HCl | 1000 |
| F6 | 5.2 | 61 | 139 | | | 125 | 675 | | 1000 |
| F7 | 5.7 | 37 | 163 | | | 125 | 675 | | 1000 |
| F8 | 5.5 | | | 100 | | 125 | 775 | 2M HCl | 1000 |
| F9 | 6.0 | | | 100 | | 125 | 775 | 2M HCl | 1000 |
| F10 | 6.5 | | | 100 | | 125 | 775 | 2M HCl | 1000 |
| F11 | 5.5 | | | 100 | 137.5 | | 762.5 | 2M Phosphoric | 1000 |
| | | | | | | | | Acid | |
| F12 | 6.0 | | | 100 | 137.5 | | 762.5 | 2M Phosphoric | 1000 |
| | | | | | | | | Acid | |
| F13 | 5.5 | | | 100 | 137.5 | | 762.5 | 0.25M Succinic | 1000 |
| | | | | | | | | Acid | |
| F14 | 5.5 | | | 100 | 137.5 | | 762.5 | 0.25M Succinic | 1000 |
| | | | | | | | | Acid | |

pH was adjusted with appropriate acid to the target pH ±0.1. The buffers were stored at 4° C. until used.

pH was adjusted with appropriate acid to the target pH ±0.1. The buffers were stored at 4° C. until used.

### Formulation Preparation

4 tubes each containing 50 ml AGS-22M6E (Lot #AGS22M6-VCE-02) were thawed in a room temperature water bath, then combined in a 250 ml bottle. 11 ml was allocated for each formulation and was added to dialysis cassettes. Cassettes were placed in beakers containing ˜40 fold excess of formulation buffer and stirred overnight at 2-8° C. Buffer was discarded and fresh buffer was added and stirred overnight at 2-8° C. Material was removed from cassettes and transferred to 50 ml tubes, the concentrations were determined and the volumes were adjusted with corresponding formulation buffer so that the final concentration was 10 mg/ml. Placebos were the corresponding buffers used for formulating the product.

### Formulation Vialing and Stoppering

Sterile filtration and filling were performed in a Baker SG600 laminar airflow hood. Formulations and placebos were sterile-filtered using aseptic technique (Millipore Millex-GV 0.22 μm PVDF syringe filters, #SLGV033RS). Sterile stoppered vials (Hollister-Stier 2-ml sterile stoppered vials, #7505ZA) were decrimped in the hood, and the stoppers were removed using aseptic technique. Vials were filled with 1.0 ml of formulated product or placebo, and then restoppered.

### Material Requirements and Sample Map

Material requirements ans sample map are as follows:

TABLE 4

| Concentration | | | | Total Fill | Total Protein | Total ml per | Total mg per |
|---|---|---|---|---|---|---|---|
| (mg/mL) | Condition | # of Vials | Fill Volume | volume | (mg) | formulation | formulation |
| 10 | 40° C. | 5 | 1 | 5.0 | 50 | | |
| 10 | 1 cycleFz/Th at −70° C./25° C. | 1 | 1 | 1.0 | 10 | | |
| 10 | 3 cycleFz/Th at −70° C./25° C. | 1 | 1 | 1.0 | 10 | | |
| 10 | 10 cycleFz/Th at −70° C. | 1 | 1 | 1.0 | 10 | | |
| 10 | Tween 20 Assay | 1 | 1 | 1.0 | 10 | | |
| 10 | RT Shake for 24hrs | 1 | 1 | 1.0 | 10 | 10 | 100 |

| | | | Total Fill | | | |
|---|---|---|---|---|---|---|
| | | | Volume | Protein In | Protein | |
| # of Prot Concs to Test | # of Buffers to Test | Total # Vials | (mL) | Vials (mg) | required (mg) | |
| 1 | 14 | 140 | 140.0 | 1400 | 1680 | Total Sample |
| | | | | | | Number: 112 |

Days at Storage Condition

| Condition | 0 | 3 | 7 | 14 |
|---|---|---|---|---|
| 40° C. | X | X | X | X |
| 1 cycleFz/Th at −70 < C/25 < C | | X | | |
| 3 cycleFz/Th at −70 < C/25 < C | | | X | |
| 10 cycleFz/Th at −70 < C/25 < C | | | | X |
| RT Shake for 24 hrs | X | | | |

### Time Point and Assays

Time point and assays are as in Table 5 below.

TABLE 5

| | | | | | 1 X Fz/Th | 3 XFz/Th | 10 XFz/Th | |
|---|---|---|---|---|---|---|---|---|
| Analytical | T = 0 | T = 3 d | T = 7 d | T = 14 d | at −70° C./ | at −70° C./ | at −70° C./ | RT Shake |
| Assay | 13 Apr. 2010 | 16 Apr. 2010 | 20 Apr. 2010 | Apr. 27, 2010 | 25° C. | 25° C. | 25° C. | for 24 hrs |
| pH | X | X | | | | | | |
| Osmolality | X | X | | | | | | |
| Visual | X | X | X | X | X | X | X | X |
| appearance | | | | | | | | |
| A280 | X | X | X | X | X | X | X | X |
| Turbidity | X | X | X | X | X | X | X | X |
| Non- | X | X | X | X | X | X | X | X |
| reduced | | | | | | | | |
| Reduced SDS- | X | X | X | X | X | X | X | X |
| PAGE | | | | | | | | |
| SE-HPLC | X | X | X | X | X | X | X | X |
| RP-HPLC | X | | | X | | | | |

| | |
|---|---|
| Potency | Provide samples for testing |

### Liquid Formulation 40° C. Stability Study Design

Formulations and placebo vials were placed upright in an incubator set to 40° C. At each time point, one active and one placebo vial for each formulation were removed from the storage conditions according to the sample map. Samples were frozen at −70° C. and batch analyzed at the end of the study. Prior to analysis, samples were thawed at RT. Sets of 3 aliquots of each sample (70 uL aliquots for each sample) were frozen at −70° C. after filter through 0.22 um filter. After analytical testing, any remaining material was stored at 2-8° C. overnight, in case re-testing was needed. After all of the assays were complete, remaining materials were then stored at −70° C. Two frozen aliquots were used for cIEF and potency assays.

### Freeze-Thaw (−70° C. Stability Study Design

One vial for each formulation (1.0 mL fill) was placed upright in a −70° C. freezer for at least 4 hours, which allowed for freezing. For thawing, each vial was removed from storage and thawed at room temperature until ice was no longer observed, and then the vial was gently swirled. This constituted one complete freeze-thaw cycle. One, three and ten freeze-thaw cycles were completed for each tested formulation sample vial. Following the final freeze-thaw cycle, all samples were evaluated by analytical testing. Sets of 3 aliquots of each sample (70 uL aliquots for each sample) were frozen at −70° C. immediately. After analytical testing, any remaining material was stored at 2-8° C. overnight, in case re-testing was needed. After all of the assays were complete, the remaining materials were stored at −70° C. Two frozen aliquots were used for cIEF and potency assays.

Agitation Study design

One vial for each formulation was secured upright in a standard freezer box. The box was then attached to an IKA-VIBRAMAX-VXR orbital shaker set at 500 rpm at room temperature for 24 hours. The samples were then removed and stored at −70° C. until analysis.

### Formulation Standard

1.2 mL of AGS-22M6E (Lot #AGS22M6-VCE-02) starting material (12.5 mg/mL in 20 mM Histidine pH 6.0 buffer containing 5% sucrose and 0.02% polysorbate-20) was taken and aliquoted at 200 ul/vial, then stored at −70° C. as formulation standard for this study.

Visual appearance, A280 (protein concentration and drug loading), A330 (turbidity), SE-HPLC, non-reduced and reduced SDS-PAGE, RP-HPLC-NPI. iCIEF and potency were used to evaluate the stability of AGS-22M6E

Visual appearance: All samples showed no color, no cloudiness and no particulates over the course of the study. No particulates were seen even upon shaking.

A280 (protein concentration) analysis: The results of the A280 analysis are shown in Table 6 below.

TABLE 6

| | A280 | | | |
|---|---|---|---|---|
| | Days at 40° C. | | | |
| Sample | 0 | 3 | 7 | 14 |
| F1 | 0.6549 | 0.6274 | 0.6805 | 0.6449 |
| F2 | 0.6818 | 0.7017 | 0.6929 | 0.6853 |
| F3 | 0.7069 | 0.6491 | 0.7130 | 0.6990 |
| F4 | 0.7056 | 0.7147 | 0.7106 | 0.7217 |
| F5 | 0.7123 | 0.6837 | 0.7138 | 0.7201 |
| F6 | 0.6616 | 0.6789 | 0.6935 | 0.6830 |
| F7 | 0.6594 | 0.6558 | 0.6672 | 0.6730 |
| F8 | 0.6986 | 0.6903 | 0.7037 | 0.7017 |
| F9 | 0.6834 | 0.7002 | 0.7063 | 0.7061 |
| F10 | 0.6888 | 0.6839 | 0.6937 | 0.7023 |
| F11 | 0.7032 | 0.7109 | 0.7074 | 0.6950 |
| F12 | 0.7040 | 0.6622 | 0.7088 | 0.7255 |
| F13 | 0.6736 | 0.6754 | 0.6874 | 0.6818 |
| F14 | 0.6944 | 0.6745 | 0.6878 | 0.7003 |

| | A280 | | | | |
|---|---|---|---|---|---|
| Sample | 0 | 1X FzTh | 3X FzTh | 10X FzTh | 24 hr Shake |
| F1 | 0.6549 | 0.6582 | 0.6951 | 0.65725 | 0.66017 |
| F2 | 0.6818 | 0.6809 | 0.6761 | 0.68321 | 0.67565 |
| F3 | 0.7069 | 0.6963 | 0.6958 | 0.69771 | 0.70847 |
| F4 | 0.7056 | 0.7048 | 0.6817 | 0.69875 | 0.68629 |
| F5 | 0.7123 | 0.7027 | 0.6961 | 0.70278 | 0.71533 |
| F6 | 0.6616 | 0.6651 | 0.6715 | 0.68515 | 0.66747 |
| F7 | 0.6594 | 0.6622 | 0.6585 | 0.63399 | 0.65046 |
| F8 | 0.6986 | 0.6969 | 0.7042 | 0.69579 | 0.69878 |
| F9 | 0.6834 | 0.6893 | 0.6876 | 0.67558 | 0.69247 |
| F10 | 0.6888 | 0.6862 | 0.6921 | 0.68071 | 0.68312 |
| F11 | 0.7032 | 0.6967 | 0.6905 | 0.68287 | 0.70169 |
| F12 | 0.7040 | 0.6892 | 0.7064 | 0.69924 | 0.68928 |
| F13 | 0.6736 | 0.6746 | 0.6745 | 0.65992 | 0.66715 |
| F14 | 0.6944 | 0.6831 | 0.7003 | 0.67608 | 0.68892 |

| | Concentration (mg/mL) | | | |
|---|---|---|---|---|
| | Days at 40° C. | | | |
| Sample | 0 | 3 | 7 | 14 |
| F1 | 9.01 | 8.63 | 9.36 | 8.87 |
| F2 | 9.38 | 9.65 | 9.53 | 9.43 |
| F3 | 9.72 | 8.93 | 9.81 | 9.62 |
| F4 | 9.71 | 9.83 | 9.77 | 9.93 |
| F5 | 9.80 | 9.40 | 9.82 | 9.90 |
| F6 | 9.10 | 9.34 | 9.54 | 9.40 |
| F7 | 9.07 | 9.02 | 9.18 | 9.26 |
| F8 | 9.61 | 9.50 | 9.68 | 9.65 |
| F9 | 9.40 | 9.63 | 9.71 | 9.71 |
| F10 | 9.47 | 9.41 | 9.54 | 9.66 |
| F11 | 9.67 | 9.78 | 9.73 | 9.56 |
| F12 | 9.68 | 9.11 | 9.75 | 9.98 |
| F13 | 9.27 | 9.29 | 9.46 | 9.38 |
| F14 | 9.55 | 9.28 | 9.46 | 9.63 |

| | Concentration (mg/mL) | | | | |
|---|---|---|---|---|---|
| Sample | 0 | 1X FzTh | 3X FzTh | 10X | 24 hr Shake |
| F1 | 9.01 | 9.05 | 9.56 | 9.04 | 9.08 |
| F2 | 9.38 | 9.37 | 9.30 | 9.40 | 9.29 |
| F3 | 9.72 | 9.58 | 9.57 | 9.60 | 9.75 |
| F4 | 9.71 | 9.69 | 9.38 | 9.61 | 9.44 |
| F5 | 9.80 | 9.67 | 9.57 | 9.67 | 9.84 |
| F6 | 9.10 | 9.15 | 9.24 | 9.42 | 9.18 |
| F7 | 9.07 | 9.11 | 9.06 | 8.72 | 8.95 |
| F8 | 9.61 | 9.59 | 9.69 | 9.57 | 9.61 |
| F9 | 9.40 | 9.48 | 9.46 | 9.29 | 9.53 |
| F10 | 9.47 | 9.44 | 9.52 | 9.36 | 9.40 |
| F11 | 9.67 | 9.58 | 9.50 | 9.39 | 9.65 |
| F12 | 9.68 | 9.48 | 9.72 | 9.62 | 9.48 |
| F13 | 9.27 | 9.28 | 9.28 | 9.08 | 9.18 |
| F14 | 9.55 | 9.40 | 9.63 | 9.30 | 9.48 |

As shown, no changes of protein concentration were observed.

A330 (turbidity) analysis: The results of the A330 analysis are shown in Table 7 below.

TABLE 7

| | A330 | | | | | |
|---|---|---|---|---|---|---|
| | Days at 40° C. | | | | | |
| | 0 | | 3 | 7 | 14 | |
| Sample | Placebo | Active | Active | Active | Placebo | Active |
| F1 | −0.0006 | 0.0591 | 0.1030 | 0.1053 | 0.0131 | 0.1223 |
| F2 | −0.0040 | 0.0727 | 0.0934 | 0.1067 | 0.0363 | 0.1070 |
| F3 | −0.0063 | 0.0585 | 0.0632 | 0.0715 | 0.0018 | 0.0736 |
| F4 | −0.0036 | 0.0601 | 0.0667 | 0.0730 | 0.0053 | 0.0757 |
| F5 | 0.0027 | 0.0659 | 0.0720 | 0.0917 | 0.0030 | 0.0823 |
| F6 | 0.0028 | 0.1018 | 0.0970 | 0.1196 | 0.0004 | 0.1269 |
| F7 | 0.0029 | 0.0705 | 0.0885 | 0.0925 | 0.0073 | 0.1031 |
| F8 | 0.0019 | 0.0620 | 0.0598 | 0.0801 | 0.0112 | 0.0860 |
| F9 | 0.0041 | 0.0681 | 0.0776 | 0.0982 | 0.0193 | 0.1046 |
| F10 | 0.0013 | 0.0628 | 0.0760 | 0.0905 | 0.0156 | 0.0886 |
| F11 | 0.0036 | 0.0773 | 0.0649 | 0.0722 | 0.0100 | 0.0782 |
| F12 | 0.0014 | 0.0652 | 0.0641 | 0.0861 | 0.0131 | 0.0796 |
| F13 | 0.0142 | 0.0655 | 0.0660 | 0.0708 | 0.0121 | 0.0936 |
| F14 | 0.0112 | 0.0659 | 0.0637 | 0.0701 | 0.0122 | 0.0857 |
| | A330 | | | | | |
| | 1X FzTh | 3X FzTh | 10X FzTh | | 24 hr Shake | |
| Sample | Active | Active | Placebo | Active | Placebo | Active |
| F1 | 0.0638 | 0.0787 | 0.0042 | 0.0755 | 0.0083 | 0.0698 |
| F2 | 0.0683 | 0.0757 | 0.0075 | 0.0774 | 0.0089 | 0.0719 |
| F3 | 0.0631 | 0.0645 | 0.0008 | 0.0620 | 0.0235 | 0.0669 |
| F4 | 0.0593 | 0.0908 | 0.0005 | 0.0600 | 0.0036 | 0.0577 |
| F5 | 0.0647 | 0.0598 | 0.0049 | 0.0685 | 0.0099 | 0.0615 |
| F6 | 0.0805 | 0.0796 | −0.0001 | 0.0728 | 0.0180 | 0.0737 |
| F7 | 0.0714 | 0.0777 | 0.0025 | 0.0745 | 0.0100 | 0.0695 |
| F8 | 0.0630 | 0.0750 | 0.0006 | 0.0696 | 0.0053 | 0.0568 |
| F9 | 0.0670 | 0.0737 | 0.0034 | 0.0715 | 0.0022 | 0.0692 |
| F10 | 0.0595 | 0.0750 | 0.0007 | 0.0752 | 0.0050 | 0.0676 |
| F11 | 0.0559 | 0.0763 | 0.0016 | 0.0694 | 0.0013 | 0.0629 |
| F12 | 0.0625 | 0.0679 | 0.0006 | 0.0695 | 0.0028 | 0.0695 |
| F13 | 0.0616 | 0.0701 | 0.0028 | 0.0820 | −0.0009 | 0.0759 |
| F14 | 0.0611 | 0.0745 | −0.0017 | 0.1033 | 0.0108 | 0.0972 |

As shown, formulations F1 and F6 exhibited the most significant increases in turbidity over time. At T=0, higher turbidity was noted for formulation F6 which was not observed in other formulations.

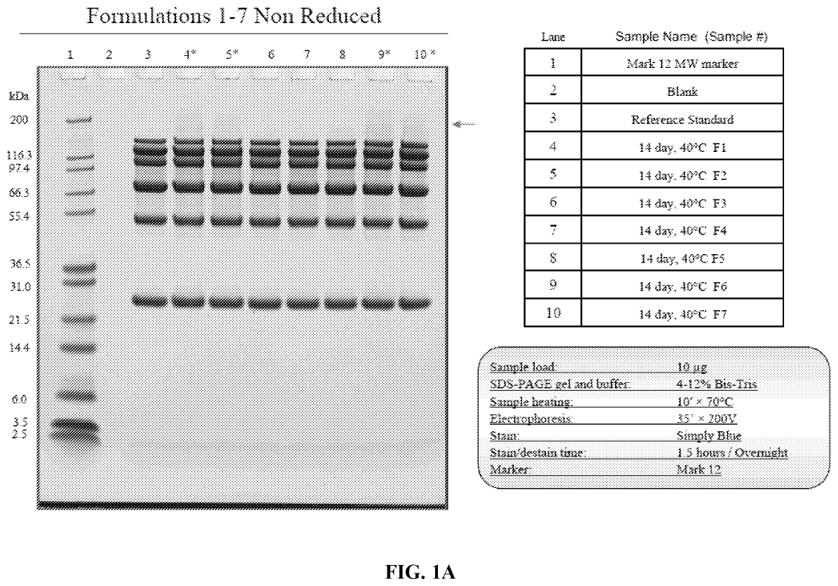

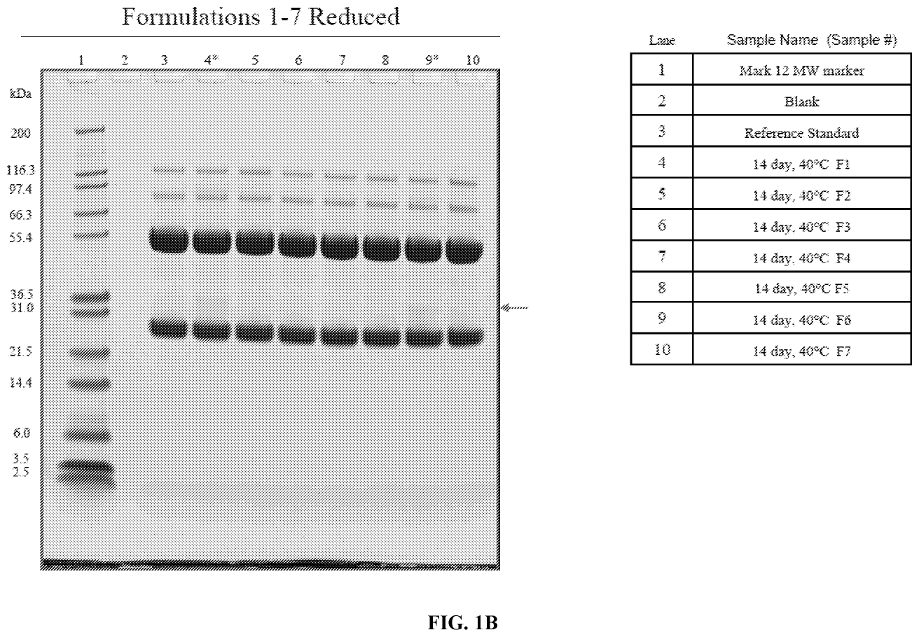

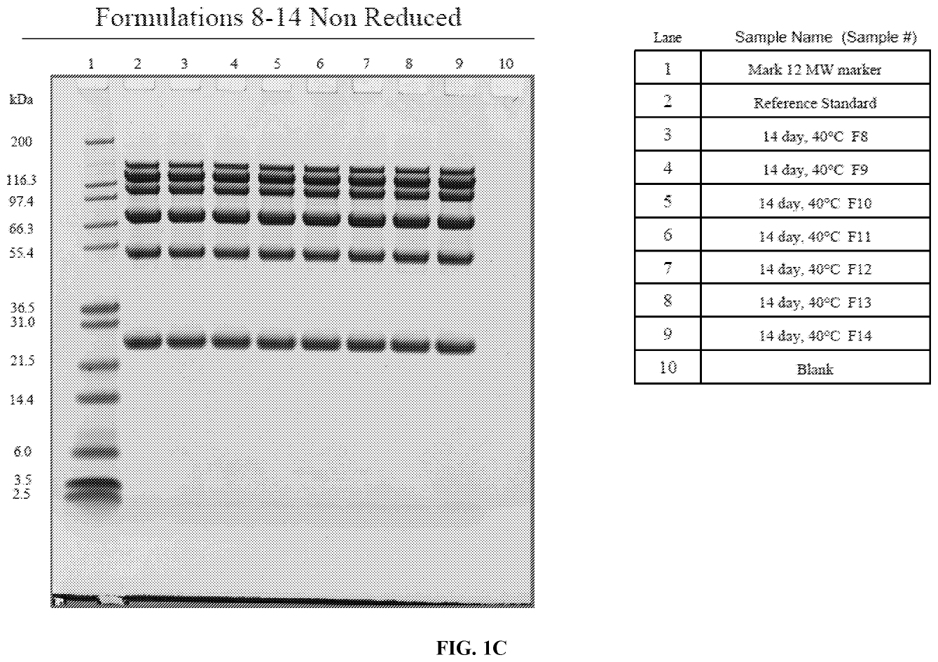

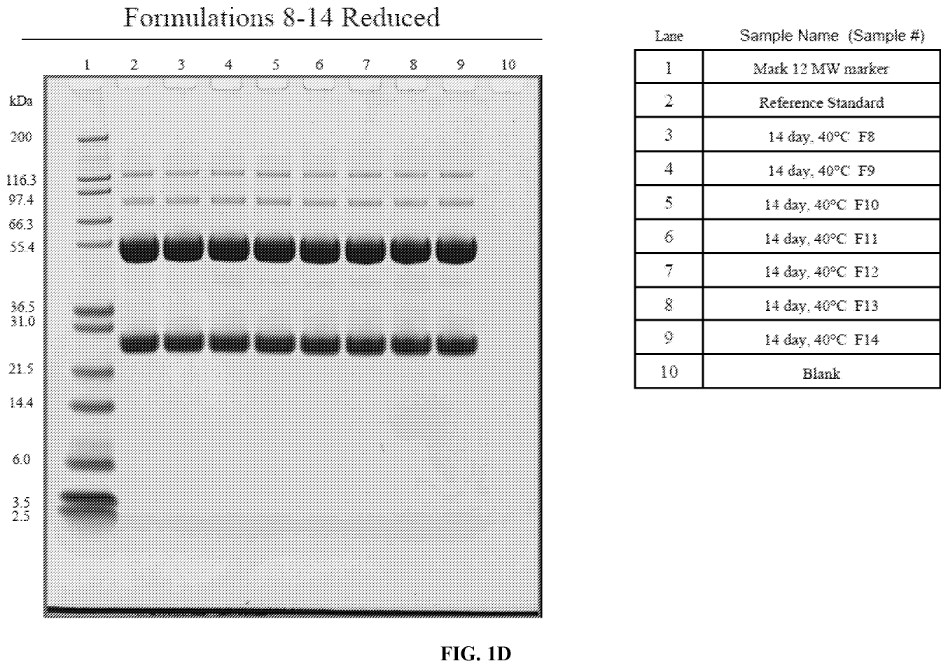

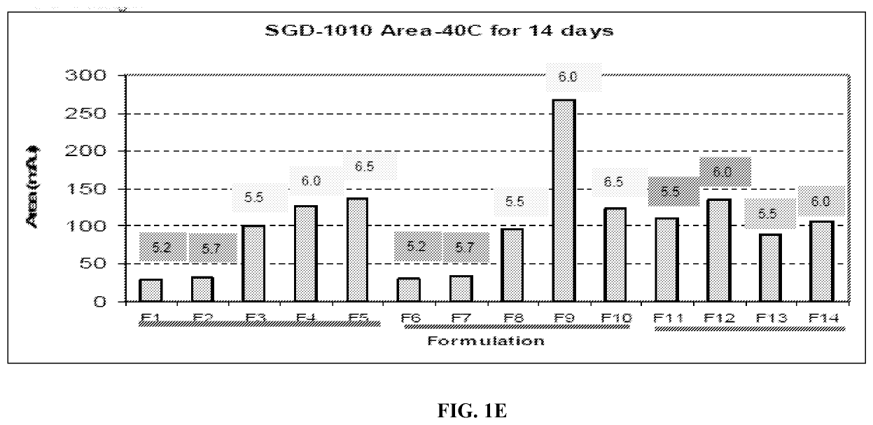

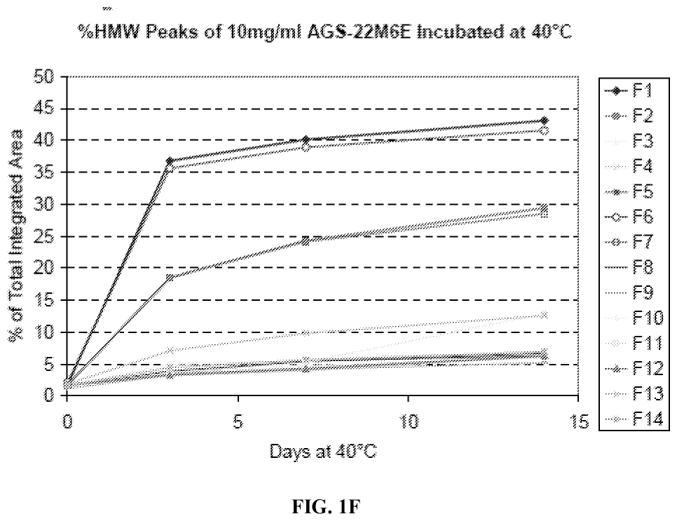

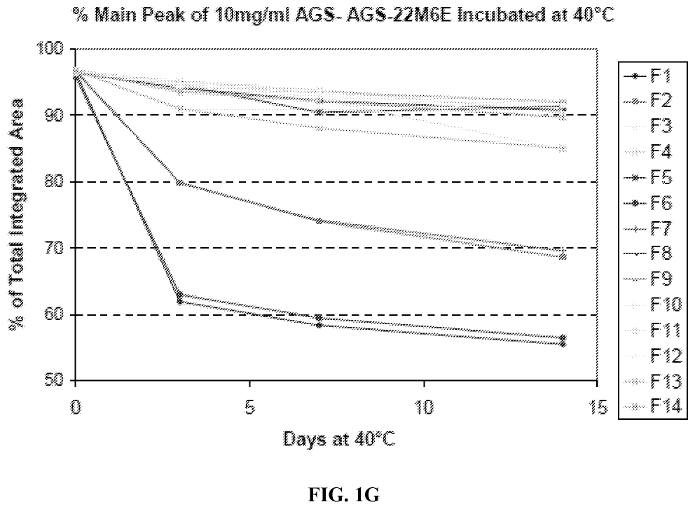

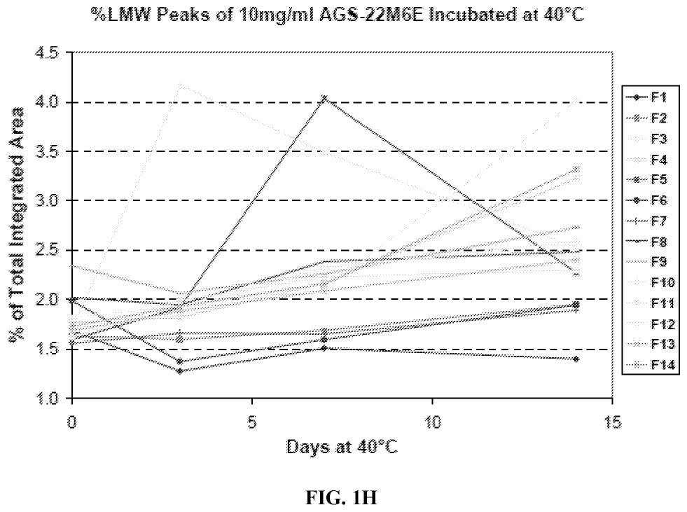

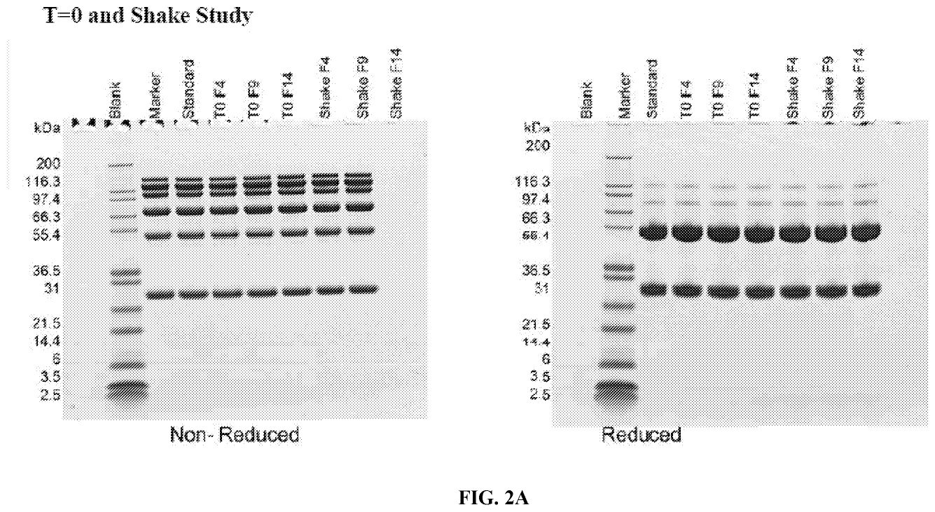

SDS-PAGE analysis: The results of the SDS-PAGE analysis are shown in FIGS. 1A, 1B, 1C, and 1D. Minor low molecular weight (LMW) bands (˜35kD) were observed by reduced SDS-PAGE in F1 and F6 after 14 days. In non-reduced SDS-PAGE analysis, F1, F2, F6 and F7 also demonstrated minor high molecular weight (HMW) bands (˜200kD) not previously present at T=0.

RP-HPLC analysis: Table 8 and FIG. 1E show the results of the RP-HPLC analysis. No free SGD1010 (trace cleavage of the drug MMAE) was detected by RP-HPLC at t=0 for any of the formulations, however after 14 days at 40° C., SGD1010 (ranging from 0.17-1.59 uM) was noted and was slightly faster at higher pH for the histidine than for the citrate formulations, with the histidine/succinic acid performing slightly better than the histidine/phosphoric and histidine/HCl.

TABLE 8

| | | | Trehalose | Sucrose | | μM |
|---|---|---|---|---|---|---|
| Formulation # | Buffer | pH | Dihydrate (%) | (%) | Area | SGD1010 |
| F1 | 20 mM sodium | 5.2 | 5.5 | 0 | 0 | 0.00 |
| | citrate/citric acid | | | | 28 | 0.17 |
| F2 | | 5.7 | | | 0 | 0.00 |
| | | | | | 31 | 0.18 |
| F3 | 20 mM | 5.5 | | | 0 | 0.00 |
| | histidine/HCl | | | | 101 | 0.60 |
| F4 | | 6 | | | 0 | 0.00 |
| | | | | | 127 | 0.76 |
| F5 | | 6.5 | | | 0 | 0.00 |
| | | | | | 136 | 0.81 |
| F6 | 20 mM sodium | 5.2 | 0 | 5 | 0 | 0.00 |
| | citrate/citric acid | | | | 30 | 0.18 |
| F7 | | 5.7 | | | 0 | 0.00 |
| | | | | | 34 | 0.20 |
| F8 | 20 mM | 5.5 | | | 0 | 0.00 |
| | histidine/HCl | | | | 96 | 0.57 |
| F9 | | 6 | | | 0 | 0.00 |
| | | | | | 267 | 1.59 |
| F10 | | 6.5 | | | 0 | 0.00 |
| | | | | | 124 | 0.74 |
| F11 | 20 mM histidine/ | 5.5 | 5.5 | 0 | 0 | 0.00 |
| | Phosphoric acid | | | | 111 | 0.66 |
| F12 | | 6 | | | 0 | 0.00 |
| | | | | | 135 | 0.80 |
| F13 | 20 mM histidine/ | 5.5 | | | 0 | 0.00 |
| | Succinic acid | | | | 89 | 0.53 |
| F14 | | 6 | | | 0 | 0.00 |
| | | | | | 106 | 0.63 |

SE-HPLC analysis: As shown in Table 9 below and FIGS. 1F, 1G, and 1H, increasing levels of HMW aggregates were evident by SE-HPLC for all formulations at pH 5.2-5.7, with the citrate formulations showing more aggregates than histidine at corresponding pH. At similar pH, citrate showed more aggregates than histidine. Histidine formulations at pH 6.0 showed better stability than those at pH 5.5 and at pH 6.5. There was no difference observed between trehalose and sucrose.

TABLE 9

| | | Main Peak | % of Total Intearated Area | | | Integrated Area | | | |
|---|---|---|---|---|---|---|---|---|---|
| | Days at | Retention | Pre | Main | Post | Pre | Main | Post | |
| Formulation | 40° C. | Time | Peaks | Peak | Peaks | Peaks | Peak | Peaks | Total |
| 1 | 0 | 19.5 | 1.8 | 96.5 | 1.7 | 83 | 4500 | 79 | 4661 |
| | 3 | 19.5 | 36.8 | 61.9 | 1.3 | 1812 | 3050 | 63 | 4924 |
| | 7 | 19.6 | 40.1 | 58.3 | 1.5 | 1953 | 2839 | 73 | 4866 |
| | 14 | 19.6 | 43.1 | 55.5 | 1.4 | 2095 | 2696 | 68 | 4858 |
| 2 | 0 | 19.5 | 1.8 | 96.6 | 1.6 | 93 | 4936 | 83 | 5112 |
| | 3 | 19.6 | 18.5 | 79.9 | 1.6 | 889 | 3832 | 77 | 4797 |
| | 7 | 19.6 | 24.3 | 74.0 | 1.7 | 1299 | 3949 | 90 | 5338 |
| | 14 | 19.6 | 29.4 | 63.6 | 1.9 | 1442 | 3363 | 95 | 4901 |
| 3 | 0 | 19.5 | 1.6 | 96.7 | 1.7 | 82 | 5078 | 89 | 5249 |
| | 3 | 19.5 | 5.0 | 90.8 | 4.2 | 253 | 4603 | 211 | 5067 |
| | 7 | 19.5 | 5.5 | 91.0 | 3.5 | 268 | 4463 | 171 | 4903 |
| | 14 | 19.5 | 6.5 | 91.0 | 2.5 | 311 | 4333 | 120 | 4764 |
| 4 | 0 | 19.5 | 1.7 | 96.5 | 1.8 | 81 | 4636 | 85 | 4802 |
| | 3 | 19.5 | 3.1 | 95.1 | 1.8 | 149 | 4571 | 87 | 4808 |
| | 7 | 19.5 | 3.9 | 93.9 | 2.2 | 201 | 4797 | 110 | 5108 |
| | 14 | 19.5 | 6.3 | 90.5 | 3.2 | 314 | 4536 | 162 | 5012 |
| 5 | 0 | 19.5 | 1.6 | 96.8 | 1.6 | 85 | 4991 | 82 | 5158 |
| | 3 | 19.5 | 3.5 | 94.6 | 1.9 | 167 | 4534 | 92 | 4793 |
| | 7 | 19.5 | 5.5 | 90.5 | 4.0 | 282 | 4622 | 206 | 5109 |
| | 14 | 19.5 | 6.3 | 91.4 | 2.3 | 308 | 4430 | 110 | 4847 |
| 6 | 0 | 19.5 | 2.2 | 95.8 | 2.0 | 97 | 4242 | 88 | 4427 |
| | 3 | 19.6 | 35.6 | 63.0 | 1.4 | 1754 | 3100 | 68 | 4921 |
| | 7 | 19.6 | 38.9 | 59.5 | 1.6 | 1908 | 2915 | 78 | 4902 |
| | 14 | 19.6 | 41.6 | 58.5 | 1.9 | 2047 | 2783 | 96 | 4926 |
| 7 | 0 | 19.5 | 1.8 | 96.7 | 1.6 | 89 | 4867 | 78 | 5034 |
| | 3 | 19.6 | 18.6 | 79.8 | 1.7 | 906 | 3895 | 81 | 4882 |
| | 7 | 19.6 | 24.2 | 74.2 | 1.7 | 1145 | 3515 | 78 | 4739 |
| | 14 | 19.6 | 28.5 | 69.6 | 1.9 | 1360 | 3318 | 90 | 4768 |
| 8 | 0 | 19.5 | 1.6 | 96.4 | 2.0 | 82 | 4968 | 104 | 5154 |
| | 3 | 19.5 | 3.9 | 94.1 | 1.9 | 184 | 4421 | 91 | 4696 |
| | 7 | 19.5 | 5.4 | 92.2 | 2.4 | 259 | 4388 | 113 | 4760 |
| | 14 | 19.5 | 6.7 | 90.8 | 2.5 | 322 | 4343 | 119 | 4785 |
| 9 | 0 | 19.6 | 1.2 | 96.5 | 2.3 | 60 | 4987 | 121 | 5168 |
| | 3 | 19.5 | 3.3 | 94.6 | 2.1 | 158 | 4504 | 98 | 4760 |
| | 7 | 19.5 | 4.2 | 93.6 | 2.3 | 209 | 4659 | 113 | 4981 |
| | 14 | 19.5 | 5.2 | 92.1 | 2.7 | 249 | 4408 | 130 | 4787 |
| 10 | 0 | 19.5 | 1.6 | 98.7 | 1.7 | 82 | 4914 | 85 | 5081 |
| | 3 | 19.5 | 3.4 | 94.8 | 1.9 | 155 | 4387 | 87 | 4629 |
| | 7 | 19.5 | 4.8 | 92.9 | 2.3 | 229 | 4466 | 111 | 4807 |
| | 14 | 19.5 | 5.9 | 91.5 | 2.6 | 276 | 4312 | 122 | 4710 |
| 11 | 0 | 19.5 | 1.6 | 96.8 | 1.6 | 82 | 4989 | 83 | 5154 |
| | 3 | 19.5 | 3.6 | 94.4 | 2.0 | 168 | 4415 | 93 | 4676 |
| | 7 | 19.5 | 5.3 | 92.5 | 2.2 | 250 | 4400 | 106 | 4756 |
| | 14 | 19.5 | 12.9 | 84.8 | 2.3 | 625 | 4107 | 111 | 4843 |
| 12 | 0 | 19.6 | 1.7 | 96.4 | 1.8 | 97 | 5340 | 101 | 5538 |
| | 3 | 19.5 | 3.3 | 94.7 | 1.9 | 159 | 4504 | 92 | 4755 |
| | 7 | 19.5 | 4.3 | 93.6 | 2.1 | 214 | 4637 | 104 | 4955 |
| | 14 | 19.5 | 6.3 | 89.7 | 4.0 | 319 | 4538 | 204 | 5060 |
| 13 | 0 | 19.5 | 1.9 | 96.7 | 1.7 | 93 | 4806 | 84 | 4969 |
| | 3 | 19.5 | 7.1 | 91.0 | 1.9 | 326 | 4184 | 86 | 4596 |
| | 7 | 19.5 | 9.8 | 88.1 | 2.1 | 456 | 4083 | 97 | 4636 |
| | 14 | 19.5 | 12.6 | 85.0 | 2.4 | 598 | 4041 | 114 | 4753 |
| 14 | 0 | 19.5 | 1.6 | 96.6 | 1.7 | 82 | 4634 | 86 | 5002 |
| | 3 | 19.5 | 4.5 | 93.6 | 1.9 | 204 | 4265 | 88 | 4557 |
| | 7 | 19.5 | 5.7 | 92.2 | 2.2 | 267 | 4342 | 102 | 4711 |
| | 14 | 19.5 | 6.9 | 89.7 | 3.3 | 349 | 4503 | 167 | 5018 |

No significant changes were observed between any of the formulations, either after 24 hrs of shaking at room temperature or after one, three and ten freeze-thaw cycles, as demonstrated by A330, SDS-PAGE and SE-HPLC (data not shown here). This provided assurance that the formulation study samples could be pulled at different time points and stored at −70° C.

Based on the results obtained from this study, formulations F4, F9 and F14 were selected as optimal among the 14 formulations tested and were therefore chosen as the 3 formulations to be further evaluated in the subsequent studies.

### 6.2 Example 2—Bulk Drug Substance (BDS) Freeze-Thaw and Shake Study

The formulations F4, F9 and F14 were prepared as described in Section 6.1 above. Each of the formulations F4, F9, and F14 was subjected to 1, 3 and 10 cycles of freezing at both −20° C. and −70° C. followed by thawing at between 20° C.-25° C. The samples were analyzed by visual inspection, concentration (A280) measurement, turbidity (A330) measurement, SE-HPLC, SDS-PAGE (R&NR). For the 10 cycles of freeze-thaw study, samples were also analyzed by RP-HPLC NPI.

The material requirements and the sample map are shown in Table 10 below:

TABLE 10

| | | | | | Total |
|---|---|---|---|---|---|
| Concentration | | # of | Fill | Total Fill | Protein |
| (mg/mL) | Condition | Vials | Volume | volume | (mg) |
| 10 | T = 0 | 1 | 3.5 | 3.5 | 35 |
| 10 | 1 cycle Fz/Th at −20° C./25° C. | 1 | 1 | 1.0 | 10 |
| 10 | 3 cycle Fz/Th at −20° C./25° C. | 1 | 1 | 1.0 | 10 |
| 10 | 10 cycle Fz/Th at −20° C./25° C. | 1 | 1 | 1.0 | 10 |
| 10 | 1 cycle Fz/Th at −70° C./25° C. | 1 | 1 | 1.0 | 10 |
| 10 | 3 cycle Fz/Th at −70° C./25° C. | 1 | 1 | 1.0 | 10 |
| 10 | 10 cycle Fz/Th at −70° C./25° C. | 1 | 1 | 1.0 | 10 |
| 10 | Shake at RT for 24 hrs | 1 | 3.5 | 3.5 | 35 |
| | | Total | Total Fill | Protein | Protein |
| # of Prot | # of Buffers | # | Volume | In Vials | required |
| Concs to Test | to Test | Vials | (mL) | (mg) | (mg) |
| 1 | 3 | 24 | 39 | 390 | 468 |

Notes:
Vials: 5 mL sterile screw-cap polycarbonate bottles (Nalgene 5-ml, #3500-05)
BDS Fz/Th: 1 mL fill in 5 mL polycarbonate bottle.
BDS shaking: 3.5 mL fill in 5 mL polycarbonate bottle.

Table 11 below lists the assays and time points.

TABLE 11

| | | 1X Fz/Th | 3X Fz/Th | 10X Fz/Th | 1X Fz/Th | 3X Fz/Th | 10X Fz/Th | |
|---|---|---|---|---|---|---|---|---|
| | | at −70° C./ | at −70° C./ | at −70° C./ | at −20° C./ | at −20° C./ | at 20° C./ | RT Shake |
| Analytical Assay | T = 0 | 25° C. | 25° C. | 25° C. | 25° C. | 25° C. | 25° C. | for 24 hrs |
| pH | X | | | | | | | |
| Osmolality | X | | | | | | | |
| Visual appearance | X | X | X | X | X | X | X | X |
| A280 | X | X | X | X | X | X | X | X |
| Turbidity (A330) | X | X | X | X | X | X | X | X |
| Non-reduced SDS-PAGE | X | X | X | X | X | X | X | X |
| Reduced SDS-PAGE | X | X | X | X | X | X | X | X |
| SE-HPLC | X | X | X | X | X | X | X | X |
| RP-HPLC-NPI | X | X | X | X | X | X | X | X |

| | | |
|---|---|---|
| HIAC | X | X |

| | |
|---|---|
| Potency &CIEF | Provide samples for testing |

An agitation study at RT for 24 hrs was also performed on each formulation and all test samples were analyzed for visual, concentration (A280), Turbidity (A330), SE-HPLC and HIAC.

Selected samples from the above studies were also used for iCIEF and Potency studies.

Formulation vialing and stoppering, agitation study design, freeze-thaw study design, and formulation standard are as described below.

### Formulation Vialing and Stoppering

Sterile filtration and filling were performed in a Baker SG600 laminar airflow hood. Formulations and placebos were sterile-filtered using aseptic technique (Millipore Millex-GV 0.22 μm PVDF syringe filters, #SLGV033RS). The filtered AGS-22M6E and filtered formulation buffers (placebo) were transferred to the sterile screw-cap polycarbonate bottles (Nalgene 5-ml, #3500-05) using a 5 mL electronic pipette with sterile tips.

### Agitation Study Design

One vial per formulation was secured upright in a standard freezer box. The box was then attached to an IKA-VIBRAMAX-VXR orbital shaker set at 500 rpm at room temperature for 24 hours. Then the samples were removed and stored at 70° C. until analysis. Sets of 3 aliquots of each sample (70 uL aliquots for each sample) were frozen at −70° C. after filter through 0.22 um filter. After analytical testing, any remaining material was stored at 2-8° C. overnight, in case re-testing was needed. After all of the assays were complete, remaining materials were then stored at −70° C. Two frozen aliquots were used for cIEF and potency assays.

Freeze-Thaw (−70° C. and −20° C. Stability Study Design [00416]1 vial per formulation (1 ml fill in 5 mLpolycarbonate bottle) was placed upright in a −70° C. and −20° C. freezer for at least 4 hours, which allowed for freezing. For thawing, each vial was removed from storage and thawed at room temperature (20-25° C.) until ice was no longer observed, then the vial was gently swirled. This constituted one complete freeze-thaw cycle. Ten freeze-thaw cycles were completed for each tested formulation sample vial. Following the final freeze-thaw cycle, all samples were evaluated by analytical testing. The samples were analyzed by the following methods: Visual appearance, A280/A248, Turbidity (A330), SE-HPLC, RP-HPLC-NPI and SDS-PAGE (R & NR). Sets of 3 aliquots of each sample (70 uL aliquots for each sample) were frozen at −70° C. after filter through 0.22 um filter. After analytical testing, any remaining material was stored at 2-8° C. overnight, in case re-testing was needed. After all of the assays were complete, remaining materials were then stored at −70° C. Two frozen aliquots were used for cIEF and potency assays.

### Formulation Standard

15 mL of AGS-22M6E starting material at 12.8 mg/mL in 5. (m Sucrose, 0.02% Tween 20, pH 6.0 was taken and aliquoted at 500 ul/vial, then stored at −70° C. as formulation standard for this study.

### Results

Visual appearance: Visual appearance for all samples were analyzed in this study and no particulates were seen, even upon shaking.

A280 and A330 analysis: A280 and A330 data for formulations subjected to different conditions in this study are summarized in Table 12 below. As shown, there was no change in protein concentration at any of the conditions of shaking or freeze-thaw. In addition, there was no increase in turbidity upon freeze-thaw or shaking.

TABLE 12

| | | | | | A280 (with | | |
|---|---|---|---|---|---|---|---|
| | | | | | Placebo | | |
| | | | | | A280 | Conc | A330 |
| Time Point | Formulation | Dilution Factor | A330 | A280 | subtracted) | (mg/mL) | Undiluted |
| T = 0, BDS | 4 | 20 | 0.012 | 0.741 | 0.728 | 10.01 | 0.083 |
| | 9 | 20 | 0.021 | 0.811 | 0.795 | 10.94 | 0.091 |
| | 14 | 20 | 0.004 | 0.820 | 0.789 | 10.86 | 0.103 |
| Shake | 4 | 20 | 0.011 | 0.749 | 0.736 | 10.12 | 0.089 |
| | 9 | 20 | 0.027 | 0.827 | 0.811 | 11.16 | 0.960 |
| | 14 | 20 | 0.001 | 0.820 | 0.789 | 10.86 | 0.107 |
| 1X −20 C. FzTh | 4 | 20 | 0.006 | 0.765 | 0.752 | 10.34 | 0.097 |
| | 9 | 20 | 0.026 | 0.812 | 0.796 | 10.96 | 0.098 |
| | 14 | 20 | 0.005 | 0.823 | 0.792 | 10.90 | 0.112 |
| 3X −20 C. FzTh | 4 | 20 | 0.003 | 0.779 | 0.766 | 10.54 | 0.096 |
| | 9 | 20 | 0.030 | 0.819 | 0.803 | 11.05 | 0.098 |
| | 14 | 20 | 0.003 | 0.821 | 0.790 | 10.87 | 0.108 |
| 10X −20 C. FzTh | 4 | 20 | 0.001 | 0.783 | 0.770 | 10.59 | 0.097 |
| | 9 | 20 | 0.031 | 0.822 | 0.806 | 11.08 | 0.109 |
| | 14 | 20 | 0.008 | 0.818 | 0.787 | 10.83 | 0.128 |
| 1X −70 C. FzTh | 4 | 20 | 0.002 | 0.780 | 0.767 | 10.55 | 0.114 |
| | 9 | 20 | 0.028 | 0.839 | 0.823 | 11.32 | 0.120 |
| | 14 | 20 | 0.001 | 0.816 | 0.785 | 10.80 | 0.108 |
| 3X −70 C. FzTh | 4 | 20 | 0.004 | 0.789 | 0.776 | 10.67 | 0.095 |
| | 9 | 20 | 0.031 | 0.846 | 0.830 | 11.42 | 0.097 |
| | 14 | 20 | 0.003 | 0.830 | 0.799 | 11.00 | 0.107 |
| 10X −70 C. FzTh | 4 | 20 | 0.006 | 0.797 | 0.784 | 10.78 | 0.091 |
| | 9 | 20 | 0.031 | 0.833 | 0.817 | 11.24 | 0.090 |
| | 14 | 20 | 0.002 | 0.827 | 0.796 | 10.95 | 0.093 |

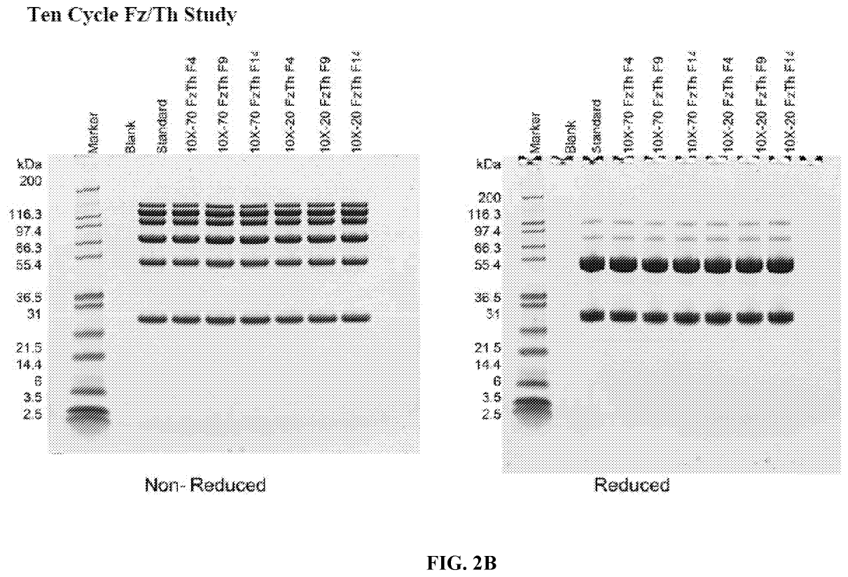

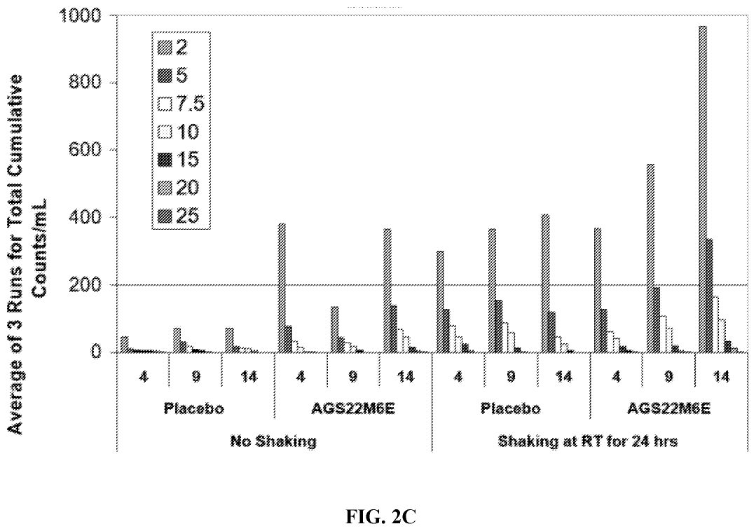

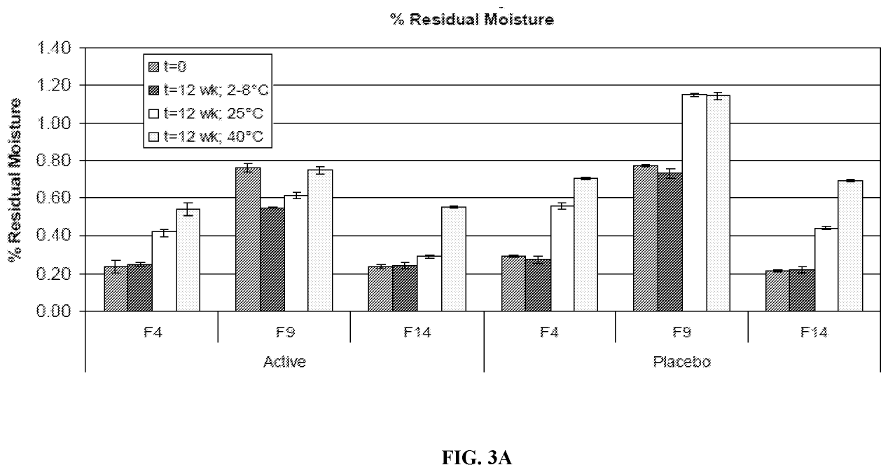

SDS-PAGE analysis: SDS-PAGE analysis results are shown in FIGS. 2A and 2B. As shown, there are no changes seen by SDS-PAGE for the shake study samples and for the freeze-thaw samples. Both reduced and non-reduced gels are comparable to the formulation standard.

RP-HPLC analysis: For the 10-cycle freeze-thaw samples, RP-HPLC analysis was performed. No evidence of SGD1010 peak was seen in any formulation as analyzed by RP-HPLC (data not shown here).

SE-HPLC analysis: The results of the SE-HPLC analysis are summarized in Table 13 below. As shown, there were no differences observed between T0 and the shake samples or the freeze-thaw samples for any of the three formulations (F4, F9, and 14) or placebos.

TABLE 13

| | | % of Total Integrated Area | | | | | | | |
|---|---|---|---|---|---|---|---|---|---|
| | | Main Peak | | | | Integrated Area | | | |
| | | Retention | | Main | Post | | Main | Post | |
| Sample Name | Condition | Time | Pre Peaks | Peak | Peaks | Pre Peaks | Peak | Peaks | Total |
| F4, BDS | T0 | 19.6 | 1.3 | 95.7 | 3.0 | 66 | 4955 | 154 | 5175 |
| | Shaking for 24 hr RT | 19.5 | 1.2 | 96.2 | 2.6 | 64 | 4937 | 134 | 5134 |
| | 1 F/T −20° C. | 19.6 | 1.2 | 95.9 | 2.9 | 63 | 4913 | 150 | 5125 |
| | 3 F/T −20° C. | 19.5 | 1.2 | 96.1 | 2.7 | 63 | 4960 | 138 | 5161 |
| | 10 F/T −20° C. | 19.5 | 1.2 | 96.3 | 2.4 | 64 | 4935 | 125 | 5123 |
| | 1 F/T −70° C. | 19.6 | 1.2 | 96.0 | 2.7 | 63 | 4929 | 141 | 5132 |
| | 3 F/T −70° C. | 19.5 | 1.2 | 96.2 | 2.5 | 64 | 5006 | 132 | 5202 |
| | 10 F/T −70° C. | 19.6 | 1.3 | 95.9 | 2.8 | 68 | 4981 | 143 | 5192 |
| F9, BDS | T0 | 19.6 | 1.3 | 95.6 | 3.1 | 66 | 5002 | 164 | 5232 |
| | Shaking for 24 hr RT | 19.6 | 1.3 | 96.4 | 2.3 | 65 | 4980 | 120 | 5164 |
| | 1 F/T −20° C. | 19.5 | 1.3 | 95.8 | 2.9 | 71 | 5007 | 152 | 5229 |
| | 3 F/T −20° C. | 19.5 | 1.2 | 96.5 | 2.3 | 63 | 4997 | 119 | 5179 |
| | 10 F/T −20° C. | 19.6 | 1.2 | 96.1 | 2.7 | 64 | 4944 | 137 | 5145 |
| | 1 F/T −70° C. | 19.6 | 1.3 | 96.3 | 2.4 | 64 | 4918 | 125 | 5107 |
| | 3 F/T −70° C. | 19.5 | 1.2 | 96.3 | 2.5 | 63 | 4978 | 127 | 5168 |
| | 10 F/T −70° C. | 19.5 | 1.2 | 96.1 | 2.6 | 65 | 4996 | 136 | 5197 |
| F14, BDS | T0 | 19.6 | 1.3 | 95.7 | 3.0 | 72 | 5190 | 161 | 5423 |
| | Shaking for 24 hr RT | 19.6 | 1.3 | 96.1 | 2.6 | 67 | 5052 | 138 | 5256 |
| | 1 F/T −20° C. | 19.6 | 1.4 | 95.7 | 2.9 | 74 | 5090 | 154 | 5319 |
| | 3 F/T −20° C. | 19.5 | 1.2 | 96.1 | 2.7 | 65 | 5047 | 142 | 5254 |
| | 10 F/T −20° C. | 19.6 | 1.3 | 96.3 | 2.4 | 66 | 5028 | 127 | 5221 |
| | 1 F/T −70° C. | 19.6 | 1.2 | 96.4 | 2.4 | 66 | 5106 | 125 | 5297 |
| | 3 F/T −70° C. | 19.5 | 1.2 | 96.3 | 2.4 | 65 | 5075 | 129 | 5269 |
| | 10 F/T −70° C. | 19.6 | 1.3 | 95.5 | 3.2 | 71 | 5096 | 169 | 5336 |

The SEC profiles obtained for each of the BDS formulations, at each of the study conditions, were analyzed (data not shown here). There was no difference noted for any of the formulations at any condition, as compared to BDS at T=0.

Table 14 below summarizes the HIAC data for the BDS samples for each of the 3 formulations tested. Comparison of results for samples prior to and after shaking at RT for 24 hrs reveals that less than 100 particles were in the range between 10p and 25 μm, with less than 2 particles being in the 25 μm range for all formulations.

TABLE 14

| | | | Average of 3 Runs for Total Cumulative Counts/mL | | | | | | |
|---|---|---|---|---|---|---|---|---|---|
| | Sample | | Size (um) | | | | | | |
| Condition | Name | Fomulation | 2 | 5 | 7.5 | 10 | 15 | 20 | 25 |
| No Shaking | Placebo | 4 | 47 | 10 | 7 | 5 | 5 | 3 | 2 |
| | | 9 | 73 | 30 | 17 | 8 | 5 | 2 | 0 |
| | | 14 | 73 | 17 | 12 | 10 | 3 | 0 | 0 |
| | AGS22M6 | 4 | 382 | 77 | 32 | 15 | 2 | 2 | 0 |
| | | 9 | 135 | 45 | 28 | 18 | 7 | 0 | 0 |
| | | 14 | 365 | 138 | 68 | 47 | 15 | 3 | 2 |
| Shaking at | Placebo | 4 | 298 | 127 | 78 | 47 | 23 | 3 | 0 |
| for 24 hrs | | 9 | 365 | 155 | 88 | 60 | 13 | 2 | 0 |
| | | 14 | 407 | 118 | 47 | 25 | 5 | 0 | 0 |
| | AGS22M6 | 4 | 368 | 127 | 62 | 42 | 18 | 5 | 2 |
| | | 9 | 557 | 192 | 108 | 73 | 20 | 3 | 2 |
| | | 14 | 967 | 333 | 165 | 97 | 32 | 12 | 2 |

Formulations F9 and F14 showed slightly elevated cumulative counts post-agitation, with F4 showing comparability pre- and post agitation, as charted in FIG. 2C (all counts for 10 and 25 um are below the USP limit).

Overall, results demonstrated that all three BDS formulations tested exhibit excellent stability under treatment of freeze-thaw cycling and agitation, and no changes were seen in any of the samples relative to T=0, by any of the analytical methods.

### 6.3 Example 3—Concurrent BDS and Drug Product (DP) Formulation Study

This study was performed in conjunction with the study described in Section 6.2 above. Formulation compositions and the materials used for this study are the same as those described in Section 6.2.

The material requirements and sample map are shown in the table below.

TABLE 15

| | | | | Total Fill | Total Protein | Total ml per | Total mg per |
|---|---|---|---|---|---|---|---|
| Concentration (mg/mL) | Condition | # of Vials | Fill Volume | volume | (mg) | formulation | formulation |
| 10 | BDS at 2-8° C. | 5 | 1 | 5.0 | 50 | | |
| 10 | BDS at −70° C. | 6 | 1 | 6.0 | 60 | | |
| 10 | DP at 2-8° C. | 8 | 5 | 40.0 | 400 | | |
| 10 | DP at 25° C./ | 6 | 5 | 30.0 | 300 | | |
| | 60% RH | | | | | | |
| 10 | DP at 40° C./ | 6 | 5 | 30.0 | 300 | 111 | 1110 |
| | 75% RH | | | | | | |

| | | | Total Fill | | |
|---|---|---|---|---|---|
| | | | Volume | Protein In | Protein |
| # of Prot Concs to Test | # of Buffers to Test | Total # Vials | (mL) | Vials (mg) | required (mg) |
| 1 | 3 | 93 | 333 | 3330 | 3996 |

The time points and assays are as described in the table below.

TABLE 16

| | Pre-Lyo | T = 0 | T = 2 wks | T = 4 wks | T = 8 wks | T = 12 wks |
|---|---|---|---|---|---|---|
| Analytical Assay | T = 0.1 | 1 Jun. 2010 | 15 Jun. 2010 | 29 Jun. 2010 | 27 Jul. 2010 | 24 Aug. 2010 |
| pH | X | X | | | | |
| Osmolality | X | X | | | | |
| Visual appearance (before | | X | X | X | X | X |
| reconstitution) | | | | | | |
| Visual appearance (BDS or | X | X | X | X | X | X |
| After reconstitution) | | | | | | |
| Reconstitution Time (DP only) | | X | X | X | X | X |
| A280 | X | X | X | X | X | X |
| Turbidity (A3 30) | X | X | X | X | X | X |
| Non-reduced SDS-PAGE | X | X | X | X | X | X |
| Reduced SDS-PAGE | X | X | X | X | X | X |
| SE-HPLC | X | X | X | X | X | X |
| RP-HPLC-NPI | X | X | X | X | X | X |
| Residual Moisture (DP only) | | X | | | | X |

| | |
|---|---|
| Potency &CIEF* | Provide samples for testing |

Note:
Samples for the liquid arm of the study were frozen at −70° C. until samples from the lyophilized arm were prepared. Frozen liquid samples were then placed at conditions at the same time as lyophilized samples were placed at conditions, so as to make t = 0 identical for both liquid and lyophilized arms of the study.

The specific lyophilization cycle parameters are outlined in the table below. After lyophilization was complete, vials were stoppered under vacuum at 50mT.

TABLE 17

| Step | | Temperature or | Time | Pressure |
|---|---|---|---|---|
| # | Step | Ramp Rate | (min | (mTorr) |
| 1 | Load/Equilibrate | 5° C. | 60 | |
| 2 | Ramp from 5° C. to 0° C. | 0.5° C./mi | 10 | |
| 3 | Hold | 0° C. | 60 | |
| 4 | Ramp from 0° C. to −45° C. | 1° C./min | 45 | |
| 5 | Hold | −45° C. | 840 | |
| 6 | Pump down | −45° C. | 60 | 50 |
| 7 | Ramp from −45° C. to − | 0.3° C./mi | 100 | 50 |
| 8 | Hold | −15° C. | 5040 | 50 |
| 9 | Ramp from −15° C. to 35 | 0.2° C./mi | 250 | 50 |
| 10 | Hold | 35° C. | 360 | 50 |
| 11 | Ramp from 35° C. to 5° C. | 0.5° C./mi | 60 | 50 |
| 12 | Hold | 5° C. hold | 60 | 50 |

### Liquid Formulation 2-8° C. and −70° C. Stability Study Design

Formulations and placebo vials were placed upright in a freezer set to −70° C. and an incubator set to 2-8° C. At each time point, one active and one placebo vial for each formulation were removed from the storage conditions according to the sample map for analytical testing. After analytical testing, any remaining material was stored at 2-8° C., in case re-testing was needed. Aliquots were stored at −70° C. and were used for cIEF and activity testing.

### Lyophilized Formulation 2-8° C., 25° C. and 40° C. Stability Study Design

Formulations and placebo vials were placed upright in an incubator set to 2-8° C., an incubator set to 25° C./60% RH and an incubator set to 40° C./75% RH. At each time point, one active and one placebo vial for each formulation were removed from storage condition according to the sample map for analytical testing. After analytical testing, any remaining material was stored at 2-8° C. in case re-testing was needed. Aliquots were stored at −70° C. and were used for cIEF and activity testing.

The formulation standard used in this study is the same as that in Section 6.2 above.

Lyophilization cycle analysis: A typical lyophilization cycle includes freezing, primary drying and secondary drying steps. During the freezing and drying process, the sublimation of ice can be followed by reference to several separate indicators, such as the readings of the thermocouple probes placed in placebo sample vials, the divergence and the later coincidence of the capacitance manometer and pirani gauge pressure readings, and the “dewpoint” measurement that tracks the change in the relative humidity in the chamber headspace.

By comparing the average product thermocouple temperature, the capacitance manometer/Pirani gauge reading difference and the dewpoint profile, it can be demonstrated that each correlates well with the others.

Stability of BDS formulations: Stability of BDS formulation at 2-8° C. and −70° C. storage conditions and at 2-8° C., 25° C. and 40° C. storage conditions were evaluated at T=0, 2, 4, 8, and 12 week time points. Liquid and reconstituted lyophilized samples were analyzed by concentration (A280), turbidity (A330), SE-HPLC, SDS-PAGE (R and NR) and RP-HPLC NPI at each time point. For lyophilized drug product (DP), osmolality was measured before lyophilization and after reconstitution at t=0 only; cake appearance and reconstitution time were measured at each time point; and Karl Fischer (residual moisture) was measured at T=0 and T=12 weeks only.

Visual appearance and reconstitution time: The cake formations for all three formulations were comparable. Both actives and placebos for all three formulations formed white, slightly cracked cakes with a shiny surface. All maintained intact structure. There were no differences between actives and placebos. There were no differences between these formulations. For all formulations stored at different conditions for 2, 4, 8 and 12 weeks, visual appearance of the cake was similar to T=0 as shown in the table below.

TABLE 18

| | | Protein | | Reconstitution | | | |
|---|---|---|---|---|---|---|---|
| | | Concentration | Cake | Time (seconds)\hat{ } | | | Visual |
| | Formulation | mg/ml | Appearance | 40° C | 25° C. | 2-8° C. | Appearance |
| 2 week | 4 | 10 | White cake, | 23 | 19 | 21 | Clear, |
| | 9 | 10 | slightly | 17 | 21 | 25 | colorless, |
| | 14 | 10 | cracked, shiny | 21 | 22 | 22 | no |
| | 4 | Placebo | surface | 22 | | | particulates |
| | 9 | | | 21 | | | |
| | 14 | | | 22 | | | |
| 4 week | 4 | 10 | White cake, | 29 | 25 | 23 | Clear, |
| | 9 | 10 | slightly | 17 | 23 | 21 | colorless, |
| | 14 | 10 | cracked, shiny | 20 | 21 | 27 | no |
| | 4 | Placebo | surface | 29 | | | particulates |
| | 9 | | | 26 | | | |
| | 14 | | | 20 | | | |
| 8 week | 4 | 10 | White cake, | 25 | 28 | 29 | Clear, |
| | 9 | 10 | slightly | 25 | 23 | 31 | colorless, |
| | 14 | 10 | cracked, shiny | 24 | 22 | 33 | no |
| | 4 | Placebo | surface | 28 | | | particulates |
| | 9 | | | 22 | | | |
| | 14 | | | 19 | | | |
| 12 week | 4 | 10 | White cake, | 22 | 28 | 24 | Clear, |
| | 9 | 10 | slightly | 20 | 22 | 27 | colorless, |
| | 14 | 10 | cracked, shiny | 23 | 25 | 23 | no |
| | 4 | Placebo | surface | 28 | | | particulates |
| | 9 | | | 20 | | | |
| | 14 | | | 28 | | | |

Moisture analysis: After lyophilization was completed, one active and one placebo vial from each formulation were allocated for residual moisture testing. As shown in the table below and FIG. 3A, the residual moistures of the actives and placebos for F4 and F14 were very close, ranging from 0.24 to 0.70%. F9 had higher residual moisture at every time point than F4 and F14.

TABLE 19

| | | % residual moisture | | | | SD | | | |
|---|---|---|---|---|---|---|---|---|---|
| | | t = 0 | t = 12 wk; 2-8° C. | t = 12 wk; 25° C. | t = 12 wk; 40° C. | T = 0 | 2-8° C. | 25° C. | 40° C. |
| Active | F4 | 0.24 | 0.25 | 0.42 | 0.54 | 0.03 | 0.01 | 0.02 | 0.03 |
| | F9 | 0.76 | 0.55 | 0.61 | 0.75 | 0.02 | 0.00 | 0.02 | 0.02 |
| | F1 | 0.24 | 0.24 | 0.29 | 0.55 | 0.01 | 0.02 | 0.01 | 0.01 |
| Placebo | F4 | 0.29 | 0.28 | 0.56 | 0.70 | 0.01 | 0.02 | 0.02 | 0.01 |
| | F9 | 0.77 | 0.73 | 1.15 | 1.14 | 0.01 | 0.03 | 0.01 | 0.02 |
| | F1 | 0.21 | 0.22 | 0.44 | 0.69 | 0.01 | 0.02 | 0.01 | 0.01 |

Results represent the average of three determinations on 1 vial

### Results Represent the Average of Three Determinations on 1 Vial

After samples were incubated at different conditions for 12 weeks, residual moistures for each formulation were tested again for both actives and placebos. The residual moistures for all three formulations stored at 2-8° C. for 12 weeks were similar to t=0. F4 and F14 had increased moisture relative to t=0 after stored at 25° C. and 40° C. for 12 weeks.

For all of the time points tested, after reconstitution with 4.7 mL of WFI, all of the reconstituted formulations and placebos were colorless and clear. No visible particles were observed. BDS samples stored at all conditions at all time points were clear and colorless too. No visible particles were observed.

A280 and osmolality analysis: Prior to filling and lyophilization, the protein concentrations of the formulations were checked in duplicate and found to within ±1 mg/mL of the target concentration of 10 mg/mL. After reconstitution with 4.7 mL of WFI, the protein concentrations of the formulations were within ±1 mg/mL of BDS before lyophilization (see Table 20 below, BDS, t=0). The osmolalities of the formulated samples and buffer were also tested in duplicate prior to filling and after reconstitution. The osmolalities before lyophilization and after reconstitution were between 187 and 194 mOsm/kg.

The protein concentration results of BDS and DP samples stored at different conditions are shown in Table 20 below and FIG. 3B. There was no change in protein concentration under most conditions. However, BDS samples stored at 2-8° C. showed an unexpected increase of protein concentration. There were two possible reasons: 1) condensation that was present in vials may not be completely incorporated after inverting. Although effort was made to capture all condensation from sides of bottles, some condensation may have been trapped in cap of bottle; 2) some evaporation of sample might occur if a bottle has a flawed thread, although this issue was not seen in a previous study in which 4 mL drug substance was filled in these bottles and stored at 2-8° C. for 12 weeks.

TABLE 20
(12 week concurrent BDS and DP formulation study A280 and concentration data)

| | A280 | | | | | Concentration (mg/mL) | | | | |
|---|---|---|---|---|---|---|---|---|---|---|
| Formulation | 0 | 2 | 4 | 8 | 12 | 0 | 2 | 4 | 8 | 12 |
| | BDS - Weeks at −70° C. | | | | | | | | | |
| 4 | 0.757 | 0.737 | 0.735 | 0.740 | 0.727 | 10.4 | 10.1 | 10.1 | 10.2 | 10.0 |
| 9 | 0.735 | 0.725 | 0.749 | 0.727 | 0.736 | 10.1 | 10.0 | 10.3 | 10.0 | 10.1 |
| 14 | 0.743 | 0.740 | 0.746 | 0.764 | 0.742 | 10.2 | 10.2 | 10.3 | 10.5 | 10.2 |
| | BDS - Weeks at 2-8° C. | | | | | | | | | |
| 4 | 0.757 | 0.749 | 0.763 | 0.801 | 0.812 | 10.4 | 10.3 | 10.5 | 11.0 | 11.2 |
| 9 | 0.735 | 0.748 | 0.776 | 0.817 | 0.918 | 10.1 | 10.3 | 10.7 | 11.2 | 12.6 |
| 14 | 0.743 | 0.759 | 0.778 | 0.823 | 0.824 | 10.2 | 10.4 | 10.7 | 11.3 | 11.3 |

| | A280 | | | | | | Concentration (mg/mL) | | | | | |
|---|---|---|---|---|---|---|---|---|---|---|---|---|
| Formulation | 0 | 0.1 | 2 | 4 | 8 | 12 | 0 | 0.1 | 2 | 4 | 8 | 12 |
| | Lyo DP - Weeks at 2- | | | | | | | | | | | |
| 4 | 0.757 | 0.757 | 0.745 | 0.778 | 0.773 | 0.747 | 10.4 | 10.4 | 10.2 | 10.7 | 10.6 | 10.3 |
| 9 | 0.735 | 0.769 | 0.743 | 0.777 | 0.782 | 0.769 | 10.1 | 10.6 | 10.2 | 10.7 | 10.8 | 10.6 |
| 14 | 0.743 | 0.800 | 0.766 | 0.815 | 0.751 | 0.769 | 10.2 | 11.0 | 10.5 | 11.2 | 10.3 | 10.6 |
| | Lyo DP - Weeks at | | | | | | | | | | | |
| 4 | 0.757 | 0.757 | 0.751 | 0.765 | 0.761 | 0.751 | 10.4 | 10.4 | 10.3 | 10.5 | 10.5 | 10.3 |
| 9 | 0.735 | 0.769 | 0.751 | 0.778 | 0.768 | 0.774 | 10.1 | 10.6 | 10.3 | 10.7 | 10.6 | 10.6 |
| 14 | 0.743 | 0.800 | 0.743 | 0.784 | 0.770 | 0.765 | 10.2 | 11.0 | 10.2 | 10.8 | 10.6 | 10.5 |
| | Lyo DP - Weeks at | | | | | | | | | | | |
| 4 | 0.757 | 0.757 | 0.746 | 0.766 | 0.758 | 0.736 | 10.4 | 10.4 | 10.3 | 10.5 | 10.4 | 10.1 |
| 9 | 0.735 | 0.769 | 0.750 | 0.776 | 0.763 | 0.766 | 10.1 | 10.6 | 10.3 | 10.7 | 10.5 | 10.5 |
| 14 | 0.743 | 0.800 | 0.772 | 0.807 | 0.771 | 0.755 | 10.2 | 11.0 | 10.6 | 11.1 | 10.6 | 10.4 |

TABLE 21
(12 week concurrent BDS and DP formulation
study osmolatity data)

| | Formulation | Reading 1 | Reading 2 | Average | Placebo |
|---|---|---|---|---|---|
| T = 0, | 4 | 193 | 194 | 194 | 191 |
| Lyo | 9 | 197 | 195 | 196 | 192 |
| | 14 | 193 | 194 | 194 | 186 |
| T = 0, | 4 | 189 | 190 | 190 | 188 |
| BDS | 9 | 189 | 190 | 190 | 186 |
| | 14 | 188 | 186 | 187 | 180 |

A330 analysis: A330 measurements of both BDS and DP reconstituted active and placebo vials are shown in Table 22 below and FIG. 3C. The A330 value for the active vial was slightly higher than the placebo, indicating that the AGS-22M6E protein contributes to the formulation turbidity. There was no significant increase in turbidity for both the active and placebo samples storage at all conditions.

TABLE 22

| Formulation | 0 | 2 | 4 | 8 | 12 |
|---|---|---|---|---|---|
| | BDS - Weeks at −70° C. | | | | |
| 4 | 0.083 | 0.085 | 0.084 | 0.101 | 0.091 |
| 9 | 0.091 | 0.090 | 0.085 | 0.096 | 0.086 |
| 14 | 0.103 | 0.094 | 0.097 | 0.104 | 0.103 |
| 4 Placebo | 0.009 | | | | |
| 9 Placebo | 0.010 | | | | |
| 14 Placebo | 0.010 | | | | |
| | BDS - Weeks at 2-8° C. | | | | |
| 4 | 0.083 | 0.089 | 0.082 | 0.104 | 0.111 |
| 9 | 0.091 | 0.090 | 0.091 | 0.103 | 0.114 |
| 14 | 0.103 | 0.099 | 0.095 | 0.111 | 0.112 |
| 4 Placebo | 0.009 | 0.003 | 0.004 | 0.017 | 0.008 |
| 9 Placebo | 0.010 | 0.009 | 0.005 | 0.018 | 0.015 |
| 14 Placebo | 0.010 | 0.005 | 0.002 | 0.022 | 0.011 |

| Formulation | 0 | 0.1 | 2 | 4 | 8 | 12 |
|---|---|---|---|---|---|---|
| | Lyo DP - Weeks at 2-8° C. | | | | | |
| 4 | 0.083 | 0.104 | 0.109 | 0.107 | 0.128 | 0.124 |
| 9 | 0.091 | 0.104 | 0.105 | 0.104 | 0.111 | 0.117 |
| 14 | 0.103 | 0.114 | 0.120 | 0.116 | 0.130 | 0.120 |
| 4 Placebo | 0.009 | 0.009 | | | | |
| 9 Placebo | 0.010 | 0.030 | | | | |
| 14 Placebo | 0.010 | 0.020 | | | | |
| | Lyo DP - Weeks at 25° C. | | | | | |
| 4 | 0.083 | 0.104 | 0.108 | 0.103 | 0.107 | 0.120 |
| 9 | 0.091 | 0.104 | 0.111 | 0.104 | 0.105 | 0.116 |
| 14 | 0.103 | 0.114 | 0.117 | 0.105 | 0.124 | 0.119 |
| 4 Placebo | 0.009 | 0.009 | | | | |
| 9 Placebo | 0.010 | 0.030 | | | | |
| 14 Placebo | 0.010 | 0.020 | | | | |
| | Lyo DP - Weeks at 40° C. | | | | | |
| 4 | 0.083 | 0.104 | 0.114 | 0.101 | 0.109 | 0.119 |
| 9 | 0.091 | 0.104 | 0.110 | 0.103 | 0.107 | 0.109 |
| 14 | 0.103 | 0.114 | 0.109 | 0.105 | 0.149 | 0.118 |
| 4 Placebo | 0.009 | 0.009 | 0.012 | 0.008 | 0.015 | 0.016 |
| 9 Placebo | 0.010 | 0.030 | 0.013 | 0.013 | 0.020 | 0.017 |
| 14 Placebo | 0.010 | 0.020 | 0.007 | 0.009 | 0.014 | 0.014 |

0 wk is before lyo (BDS t = 0)
0.1 wk is after lyo (previously labeled as t = 0 lyo)

SDS-PAGE analysis: FIGS. 3D and 3E show the SDS-PAGE analysis for BDS T=0 (pre-lyophilization) and T=12 week samples at 2-8° C. and −70° C. storage conditions. FIG. 3F, 3G, 3H showed the SDS-PAGE analysis for DP at T=0 and T=12 week samples at 2-8° C., 25° C. and 40° C. storage conditions. No obvious changes were seen for all of the formulations after 12 weeks stored at all conditions.

RP-HPLC analysis: All of the BDS and DP samples in this study were also tested by RP-HPLC NPI method. There was no SGD1010 peak observed in any formulation at any condition (data not shown here). For all of the controls ran at each time point, the spike recoveries of SGD1010 in the formulation standard were about 100%. At T=4 week, 8 week, and 12 week time points, although a new dilution from 10 mM SGD1010 stock was used with freshly prepared diluent, the SGD1010 peak split, and the SGD1010 spiked into the formulation standard did not split (data not shown here). This was consistent throughout the sequence (data not shown here). The calculated recovery used the combined area of the split peaks.

SE-HPLC analysis: Table 23 below summarizes SE-HPLC data for the formulation standard run at each time point for this study. The data show the level of variation in percentages of main peak and post peaks across different runs for the same sample.

TABLE 23

| | | |
|---|---|---|
| Formulation Standard | % of Total Integrated Area | Integrated Area |

| | | Main Peak | | Main | Post | | Main | Post | |
|---|---|---|---|---|---|---|---|---|---|
| Weeks | Injection | Retention | Pre Peaks | Peak | Peaks | Pre Peaks | Peak | Peaks | Total |
| 0 wk | 1 | 19.6 | 1.3 | 95.8 | 2.9 | 423 | 30906 | 924 | 32253 |
| | 2 | 19.6 | 1.3 | 95.8 | 2.9 | 404 | 30758 | 934 | 32096 |
| | 3 | 19.5 | 1.3 | 96.0 | 2.7 | 410 | 30915 | 876 | 32200 |
| | avg | 19.5 | 1.3 | 95.9 | 2.8 | 412 | 30860 | 911 | 32183 |
| | % CV | 0.0 | 2.2 | 0.1 | 3.5 | 2.4 | 0.3 | 3.4 | 0.2 |
| 2 wk | 1 | 20.7 | 1.2 | 95.7 | 3.1 | 422 | 32516 | 1047 | 33986 |
| | 2 | 19.8 | 1.2 | 95.7 | 3.0 | 403 | 31099 | 986 | 32488 |
| | 3 | 19.7 | 1.2 | 95.7 | 3.1 | 405 | 31201 | 1007 | 32613 |
| | avg | 19.7 | 1.2 | 95.7 | 3.1 | 410 | 31606 | 1013 | 33029 |
| | % CV | 0.0 | 0.1 | 0.0 | 1.0 | 2.6 | 2.5 | 3.1 | 2.5 |
| 4 wk | 1 | 19.8 | 1.2 | 95.3 | 3.6 | 379 | 30322 | 1133 | 31834 |
| | 2 | 19.8 | 1.2 | 95.4 | 3.4 | 382 | 30449 | 1100 | 31931 |
| | 3 | 19.8 | 1.2 | 95.5 | 3.3 | 400 | 30918 | 1066 | 32384 |
| | avg | 19.8 | 1.2 | 95.4 | 3.4 | 387 | 30563 | 1100 | 32050 |
| | % CV | 0.0 | 2.0 | 0.1 | 3.9 | 3.0 | 1.0 | 3.0 | 0.9 |
| 8 wk | 1 | 20.2 | 1.2 | 95.5 | 3.3 | 411 | 31427 | 1074 | 32911 |
| | 2 | 20.2 | 1.2 | 95.6 | 3.1 | 407 | 31417 | 1033 | 32857 |
| | 3 | 20.1 | 1.3 | 95.7 | 3.0 | 417 | 31657 | 999 | 33073 |
| | avg | 20.2 | 1.2 | 95.6 | 3.1 | 412 | 31500 | 1035 | 32947 |
| | % CV | 0.1 | 0.8 | 0.1 | 3.9 | 1.2 | 0.4 | 3.6 | 0.3 |
| total | avg | 19.9 | 1.2 | 95.6 | 3.1 | 405 | 31132 | 1015 | 32552 |
| | stdev | 0.3 | 0.0 | 0.21 | 0.2 | 13.8 | 585.6 | 76.3 | 596.1 |
| | % CV | 1.7 | 2.6 | 0.22 | 7.7 | 3.4 | 1.9 | 7.5 | 1.8 |

| | | | % of Total Integrated Area | | | Integrated Area | | | |
|---|---|---|---|---|---|---|---|---|---|
| | | Main Peak | | Main | Post | | Main | Post | |
| Sample | Weeks | Retention | Pre Peaks | Peak | Peaks | Pre Peaks | Peak | Peaks | Total |
| Formulation | 0 | 19.5 | 1.3 | 95.9 | 2.8 | 412.1 | 30859.6 | 911.3 | 32183 |
| Standard | 2 | 19.7 | 1.2 | 95.7 | 3.1 | 409.9 | 31605.7 | 1013.4 | 33029 |
| Average | 4 | 19.8 | 1.2 | 95.4 | 3.4 | 386.7 | 30562.9 | 1100.0 | 32050 |
| | 8 | 20.2 | 1.2 | 95.6 | 3.1 | 411.6 | 31500.2 | 1035.2 | 32947 |
| | 12 | 19.4 | 1.2 | 95.1 | 3.6 | 384.5 | 29485.9 | 1125.2 | 30996 |

Table 24 below outlines the SE-HPLC data summarizing the percentage of HMW peaks, main peak and LMW peaks for BDS samples stored at 2-8° C. and −70° C. conditions for 12 Weeks (data not shown here). The variations of percentages of main peak and post peaks at different time points were very similar to the variations seen in formulation standard (data not shown here). Therefore, it can be concluded that no major changes occurred and that all 3 formulations were stable after 12 weeks at 2-8° and −70° C. in liquid form. No major differences were observed among the formulations.

TABLE 24

| | | | % of Total Integrated Area | | | Integrated Area | | | |
|---|---|---|---|---|---|---|---|---|---|
| | Weeks at | Main Peak | | Main | Post | | Main | Post | |
| Sample Name | 2-8° C. | Retention | Pre Peaks | Peak | Peaks | Pre Peaks | Peak | Peaks | Total |
| F4 | 0 | 19.6 | 1.3 | 95.9 | 2.9 | 412 | 31291 | 940 | 32642 |
| | 2 | 19.7 | 1.3 | 95.6 | 3.1 | 445 | 32429 | 1056 | 33930 |
| | 4 | 19.8 | 1.3 | 95.4 | 3.3 | 425 | 32200 | 1119 | 33744 |
| | 8 | 20.2 | 1.3 | 95.9 | 2.8 | 479 | 35060 | 1032 | 36571 |
| | 12 | 19.4 | 1.3 | 95.3 | 3.4 | 449 | 33754 | 1216 | 35419 |
| F9 | 0 | 19.6 | 1.3 | 95.9 | 2.8 | 413 | 31522 | 932 | 32866 |
| | 2 | 19.7 | 1.3 | 95.6 | 3.1 | 433 | 32137 | 1037 | 33607 |
| | 4 | 19.8 | 1.3 | 95.5 | 3.2 | 438 | 32540 | 1100 | 34078 |
| | 8 | 20.1 | 1.3 | 95.9 | 2.8 | 493 | 35546 | 1044 | 37082 |
| | 12 | 19.4 | 1.3 | 95.4 | 3.3 | 494 | 36686 | 1285 | 38465 |
| F14 | 0 | 19.6 | 1.3 | 95.8 | 2.9 | 439 | 32730 | 981 | 34150 |
| | 2 | 19.7 | 1.3 | 95.6 | 3.1 | 444 | 31954 | 1021 | 33418 |
| | 4 | 19.8 | 1.3 | 95.5 | 3.2 | 461 | 33355 | 1120 | 34936 |
| | 8 | 20.1 | 1.4 | 95.9 | 2.8 | 503 | 35252 | 1013 | 36768 |
| | 12 | 19.4 | 1.3 | 95.5 | 3.2 | 473 | 34069 | 1125 | 35667 |
| | | | % of Total Integrated Area | | | Integrated Area | | | |
| | Weeks at | Main Peak | | Main | Post | | Main | Post | |
| Sample Name | 70° C. | Retention | Pre Peaks | Peak | Peaks | Pre Peaks | Peak | Peaks | Total |
| F4 | 0 | 19.6 | 1.3 | 95.9 | 2.9 | 412 | 31291 | 940 | 32642 |
| | 2 | 19.7 | 1.3 | 95.5 | 3.2 | 434 | 31610 | 1069 | 33112 |
| | 4 | 19.8 | 1.3 | 95.4 | 3.3 | 427 | 31179 | 1070 | 32677 |
| | 8 | 20.1 | 1.3 | 95.9 | 2.8 | 439 | 31922 | 941 | 33302 |
| | 12 | 19.4 | 1.3 | 95.4 | 3.3 | 396 | 29995 | 1041 | 31432 |
| F9 | 0 | 19.6 | 1.3 | 95.9 | 2.8 | 413 | 31522 | 932 | 32866 |
| | 2 | 19.7 | 1.3 | 95.6 | 3.1 | 429 | 31119 | 1019 | 32567 |
| | 4 | 19.8 | 1.3 | 95.4 | 3.3 | 426 | 31384 | 1079 | 32889 |
| | 8 | 20.1 | 1.3 | 95.9 | 2.8 | 442 | 32178 | 938 | 33559 |
| | 12 | 19.4 | 1.3 | 95.4 | 3.3 | 397 | 30027 | 1051 | 31475 |
| F14 | 0 | 19.6 | 1.3 | 95.8 | 2.9 | 439 | 32730 | 981 | 34150 |
| | 2 | 19.7 | 1.3 | 95.6 | 3.1 | 431 | 31071 | 1000 | 32502 |
| | 4 | 19.8 | 1.3 | 95.4 | 3.3 | 437 | 31571 | 1097 | 33105 |
| | 8 | 20.1 | 1.3 | 95.9 | 2.8 | 454 | 32541 | 952 | 33947 |
| | 12 | 19.4 | 1.3 | 95.4 | 3.3 | 412 | 30512 | 1046 | 31970 |

Table 25 below outlines the SE-HPLC data, which summarizes the percentage of HM/W peaks, main peak and LMW peaks for lyophilized AGS-22M6E stored at 2-8° C., 25° C./600% RH and 40° C./750% RH conditions for 12 Weeks. FIG. 3I shows SE-HPLC data in graphs for AGS-22M6E BDS and DP stored at different conditions. The variations of percentages of main peak and post peaks at different time points were very similar to the variations seen in formulation standard, indicating that no major changes occurred in the formulation samples. The SE-HPLC overlays for different DP formulations stored at different conditions were also analyzed (data not shown here). There were no differences observed before and after lyophilization. All 3 formulations were stable after 12 weeks at 2-8%, 25%, and 40° C. in lyophilized form. All formulations behaved similarly and gave high quality product after lyophilization, in addition to acceptable stability profiles seen for the corresponding BDS in each case, both at T=0 and after shaking and freeze-thaw studies.

TABLE 25

| | | Main Peak | % of Total Integrated Area | | | Integrated Area | | | |
|---|---|---|---|---|---|---|---|---|---|
| | Weeks at | Retention | | Main | Post | | Main | Post | |
| Sample Name | 2-8° C. | Time | Pre Peaks | Peak | Peaks | Pre Peaks | Peak | Peaks | Total |
| F4 | 0 | 19.6 | 1.3 | 95.9 | 2.9 | 412 | 31291 | 940 | 32642 |
| | 0.1 | 19.6 | 1.2 | 95.8 | 2.9 | 418 | 32092 | 972 | 33483 |
| | 2 | 19.7 | 1.2 | 95.7 | 3.1 | 400 | 31564 | 1027 | 32991 |
| | 4 | 19.8 | 1.2 | 95.6 | 3.2 | 413 | 33168 | 1106 | 34687 |
| | 8 | 20.1 | 1.3 | 95.8 | 2.9 | 449 | 32603 | 997 | 34049 |
| | 12 | 19.4 | 1.3 | 95.2 | 3.5 | 403 | 30358 | 1124 | 31885 |
| F9 | 0 | 19.6 | 1.3 | 95.9 | 2.8 | 413 | 31522 | 932 | 32866 |
| | 0.1 | 19.6 | 1.2 | 95.9 | 2.9 | 418 | 32453 | 972 | 33842 |
| | 2 | 19.8 | 1.3 | 95.5 | 3.2 | 432 | 31657 | 1068 | 33157 |
| | 4 | 19.8 | 1.3 | 95.4 | 3.3 | 441 | 32915 | 1145 | 34502 |
| | 8 | 20.1 | 1.3 | 95.8 | 2.9 | 459 | 33035 | 1000 | 34494 |
| | 12 | 19.4 | 1.3 | 95.2 | 3.5 | 411 | 31112 | 1149 | 32672 |
| F14 | 0 | 19.6 | 1.3 | 95.8 | 2.9 | 439 | 32730 | 981 | 34150 |
| | 0.1 | 19.6 | 1.3 | 95.8 | 2.9 | 450 | 33238 | 1005 | 34693 |
| | 2 | 19.8 | 1.3 | 95.4 | 3.2 | 461 | 32915 | 1110 | 34486 |
| | 4 | 19.8 | 1.3 | 95.4 | 3.3 | 472 | 34323 | 1178 | 35972 |
| | 8 | 20.1 | 1.3 | 95.8 | 2.9 | 468 | 33414 | 999 | 34881 |
| | 12 | 19.4 | 1.3 | 95.3 | 3.4 | 424 | 31239 | 1108 | 32771 |
| | | Main Peak | % of Total Integrated Area | | | Integrated Area | | | |
| | Weeks at | Retention | | Main | Post | | Main | Post | |
| Sample Name | 25° C. | Time | Pre Peaks | Peak | Peaks | Pre Peaks | Peak | Peaks | Total |
| F4 | 0 | 19.6 | 1.3 | 95.9 | 2.9 | 412 | 31291 | 940 | 32642 |
| | 0.1 | 19.6 | 1.2 | 95.8 | 2.9 | 418 | 32092 | 972 | 33483 |
| | 2 | 19.8 | 1.2 | 95.6 | 3.2 | 406 | 31761 | 1069 | 33236 |
| | 4 | 19.8 | 1.2 | 95.4 | 3.3 | 410 | 32451 | 1138 | 33999 |
| | 8 | 20.2 | 1.3 | 95.7 | 3.0 | 431 | 32611 | 1019 | 34061 |
| | 12 | 19.4 | 1.3 | 95.2 | 3.5 | 420 | 30805 | 1142 | 32367 |
| F9 | 0 | 19.6 | 1.3 | 95.9 | 2.8 | 413 | 31522 | 932 | 32866 |
| | 0.1 | 19.6 | 1.2 | 95.9 | 2.9 | 418 | 32453 | 972 | 33842 |
| | 2 | 19.7 | 1.3 | 95.4 | 3.3 | 438 | 31863 | 1089 | 33389 |
| | 4 | 19.8 | 1.3 | 95.4 | 3.4 | 440 | 32810 | 1155 | 34405 |
| | 8 | 20.2 | 1.3 | 95.7 | 3.0 | 454 | 32933 | 1015 | 34402 |
| | 12 | 19.4 | 1.3 | 95.2 | 3.5 | 417 | 31201 | 1153 | 32770 |
| F14 | 0 | 19.6 | 1.3 | 95.8 | 2.9 | 439 | 32730 | 981 | 34150 |
| | 0.1 | 19.6 | 1.3 | 95.8 | 2.9 | 450 | 33238 | 1005 | 34693 |
| | 2 | 19.7 | 1.3 | 95.4 | 3.2 | 456 | 32561 | 1104 | 34121 |
| | 4 | 19.8 | 1.3 | 95.3 | 3.3 | 470 | 33734 | 1180 | 35383 |
| | 8 | 20.1 | 1.4 | 95.7 | 3.0 | 480 | 33192 | 1027 | 34698 |
| | 12 | 19.4 | 1.3 | 95.2 | 3.4 | 445 | 31605 | 1142 | 33192 |
| | | Main Peak | % of Total Integrated Area | | | Integrated Area | | | |
| | Weeks at | Retention | | Main | Post | | Main | Post | |
| Sample Name | 40° C. | Time | Pre Peaks | Peak | Peaks | Pre Peaks | Peak | Peaks | Total |
| F4 | 0 | 19.6 | 1.3 | 95.9 | 2.9 | 412 | 31291 | 940 | 32642 |
| | 0.1 | 19.6 | 1.2 | 95.8 | 2.9 | 418 | 32092 | 972 | 33483 |
| | 2 | 19.8 | 1.3 | 95.4 | 3.4 | 414 | 31498 | 1120 | 33032 |
| | 4 | 19.8 | 1.3 | 95.4 | 3.3 | 448 | 32735 | 1144 | 34327 |
| | 8 | 20.2 | 1.4 | 95.5 | 3.1 | 455 | 32159 | 1045 | 33659 |
| | 12 | 19.4 | 1.4 | 95.1 | 3.5 | 450 | 30523 | 1137 | 32109 |
| F9 | 0 | 19.6 | 1.3 | 95.9 | 2.8 | 413 | 31522 | 932 | 32866 |
| | 0.1 | 19.6 | 1.2 | 95.9 | 2.9 | 418 | 32453 | 972 | 33842 |
| | 2 | 19.8 | 1.3 | 95.4 | 3.3 | 428 | 32091 | 1103 | 33622 |
| | 4 | 19.8 | 1.2 | 95.6 | 3.2 | 414 | 32985 | 1120 | 34518 |
| | 8 | 20.2 | 1.3 | 95.6 | 3.1 | 426 | 32104 | 1039 | 33569 |
| | 12 | 19.4 | 1.3 | 95.1 | 3.6 | 422 | 30813 | 1158 | 32393 |
| F14 | 0 | 19.6 | 1.3 | 95.8 | 2.9 | 439 | 32730 | 981 | 34150 |
| | 0.1 | 19.6 | 1.3 | 95.8 | 2.9 | 450 | 33238 | 1005 | 34693 |
| | 2 | 19.7 | 1.3 | 95.5 | 3.2 | 436 | 32383 | 1099 | 33918 |
| | 4 | 19.8 | 1.3 | 95.5 | 3.2 | 450 | 33290 | 1126 | 34866 |
| | 8 | 20.2 | 1.5 | 95.5 | 3.0 | 507 | 32935 | 1044 | 34486 |
| | 12 | 19.4 | 1.5 | 95.1 | 3.5 | 475 | 30996 | 1132 | 32604 |

### 6.4 Example 4-Lyophilization Cycle Development

Formulation F4 described in above sections was selected for this further lyophilization cycle development study. The lyophilization cycle parameters are shown in the table below. After lyophilization was complete, vials were stoppered under vacuum at 50mT. The tray of vials was removed from the lyophilizer and vials were individually crimped with aluminum seals.

TABLE 26

| Step | | Temperature or | Time | Pressure |
|---|---|---|---|---|
| # | Step | Ramp Rate | (min) | (mTorr) |
| 1 | Load/Equilibrate | 5° C. | 60 | |
| 2 | Ramp from 5° C. to 0° C. | 0.5° C./min | 10 | |
| 3 | Hold | 0° C. | 60 | |
| 4 | Ramp from 0° C. to −45° C. | 1° C./min | 45 | |
| 5 | Hold | −45° C. | 120 | |
| 6 | Pump down | −45° C. | 60 | 50 |
| 7 | Ramp from −45° C. to −15° C. | 0.3° C./min | 100 | 50 |
| 8 | Hold | −15° C. | 2710 | 50 |
| 9 | Ramp from −15° C. to 35° C. | 0.2° C./min | 250 | 50 |
| 10 | Hold | 35° C. | 420 | 50 |
| 11 | Ramp from 35° C. to 5° C. | 0.5° C./min | 60 | 50 |
| 12 | Hold | 5° C. hold | 60 | 50 |

### Lyophilized Formulation Study Design

For each fill volume configuration, 5 product vials were filled in addition to 5 placebo vials. 2 vials from each of the active product and placebo were tested for each fill volume at T=0 (1 vial was tested for residual moisture and 1 vial was reconstituted and tested using the assays described in the study summary). In addition, 2 vials from each of active and placebo were probed for temperature monitoring during the freeze-drying process.

The formulation standard used for this study was the AGS-22M6E starting material at 12.8 mg/mL in 5.0% Sucrose, 0.02% Tween 20, pH 6.0.

### Results

Lyophilization cycle analysis: The total conservative cycle time was 2.6 days, which is significantly shorter than the 4.7 day cycle time established for a 5 mL fill volume (see Section 6.3 above). The 2.6 day cycle time however for the present study is not representative of an optimized cycle time for either fill volume. Upon careful evaluation of the individual readouts associated with the cycle, an estimate of an optimized cycle time for either fill volume could be made. Since the dew point monitor is responding to the moisture evolving from both fill volumes, it was not used in this study to estimate a more optimal drying time for the 3.0 mL or 1.5 mL fill volume. Similarly, the Pirani Gauge, although normally an excellent indicator of end point of primary drying time, in this instance, only provides a guide as it too is responding to the sublimation of ice from all vials on the shelf. Instead, for the purpose of this study, monitoring product temperature provides the most reliable tool for estimating the drying times for each individual fill volume. In this study, thermocouple probes were placed in both active and placebo vials and close concordance between product temperature profiles for active and placebo was observed for both fill volumes.

With this assurance that product temperature gives accurate measurement of drying time, comparison of data for the two fill volumes (data not shown here) demonstrates that the 1.5 mL fill volume dries in approximately 1.3 days, which is considerably faster than the 3.0 mL fill volume which dries in approximately 1.8 days.

Cake appearance: All cakes were perfect cakes, very slightly contracted, with a shiny surface. They all maintained intact structure. In most vials, there were no cracks in the cakes. When the vials were inverted, the cakes did not remain adherent to the vials, but tumbled intact in the vials. In some vials, fine cracks were observed around the edge of the meniscus circle where the cake was attached to the vial. There was no difference between the cake formation for actives and placebos, for both fill volumes. The cake appearance was also comparable to the cake appearance recorded for 5 mL fill volume lyophilized product in the study of Section 6.3.

A280 analysis: Prior to filling and lyophilization, the protein concentration of the formulation was checked in duplicate and found to be close to the target concentration of 10 mg/mL. After reconstitution with 2.8 mL and 1.4 mL of WFI respectively, the protein concentrations for the 3.0 mL and 1.5 mL fill formulations were also close to those before lyophilization (see the table below).

TABLE 27

| | Dilution | | | | |
|---|---|---|---|---|---|
| | Factor | A330 | A280 | [protein] | Average |
| Sample | (df) | nm | nm | mg/mL | (mg/mL) |
| Pre-lyo | 20 | 0.00026 | 0.73176 | 10.06 | 10.1 |
| | | 0.00000 | 0.74046 | 10.19 | |
| F4 10 mg/mL, | 20 | 0.00126 | 0.82283 | 11.30 | 11.3 |
| T = 0, 3 mL | | 0.00000 | 0.81551 | 11.22 | |
| F4 10 mg/mL, | 20 | 0.00429 | 0.78214 | 10.70 | 10.7 |
| T = 0, 1.5 mL | | | | | |

Residual moisture, reconstitution time, A330, osmolality and visual appearance: After lyophilization was complete, one vial of each fill volume from the active and placebo vials was allocated for residual moisture testing. As shown in Table 28 below, the residual moistures of the actives ranged from 0.18% for the 3.0 mL fill to 0.29% for the 1.5 mL fill. Residual moistures were determined to be slightly higher for the placebos, at 0.34% for both fill volumes. Reconstitution times were less than 1 min for all vials tested, with slightly higher times recorded for the 3.0 mL fill volumes (average 36s) compared to the 1.5 mL fill volume samples (average 20s). There was no appreciable difference observed in reconstitution time between active and placebo vials. Similarly, visual appearance for all reconstituted samples, both active and placebo, was reported as clear and colorless with no particulates.

TABLE 28

| | | Reconstitution Time\hat{ } | | | | Osmolality | | | |
|---|---|---|---|---|---|---|---|---|---|
| | Lyo Fill | (sec) | | Turbidity (A330) | | (mOsm/ky)¹ | | % Residual Moisture* | |
| Condition | Volume | Active | Placebo | Active | Placebo | Active | Placebo | Active | Placebo |
| T = 0 | 3.0 | 38 | 35 | 0.0645 | 0.0139 | 194 | 186 | 0.18 | 0.34 |
| | 1.5 | 22 | 18 | 0.0555 | 0.0097 | 190 | 189 | 0.29 | 0.34 |
| Pre-Lyo | | | | 0.0610 | 0.0142 | 190 | 181 | | |
| | | | | 0.0492 | 0.0089 | | | | |

The turbidity (A330) measurements of active and placebo vials are also shown in the above table. As shown, for both pre- and post lyophilization samples, the A330 values for the active vial was slightly higher than the placebo, confirming earlier results discussed in Section 6.3, where AGS-22M6E was shown to contribute to the formulation turbidity.

The osmolalities of the formulated samples and buffer were tested in duplicate prior to filling and after reconstitution. Table 28 above summarizes this data, showing that there was no appreciable difference in osmolality before lyophilization or after reconstitution. They were also comparable to the osmolality determined for the 5.0 mL fill configuration samples in Section 6.3.

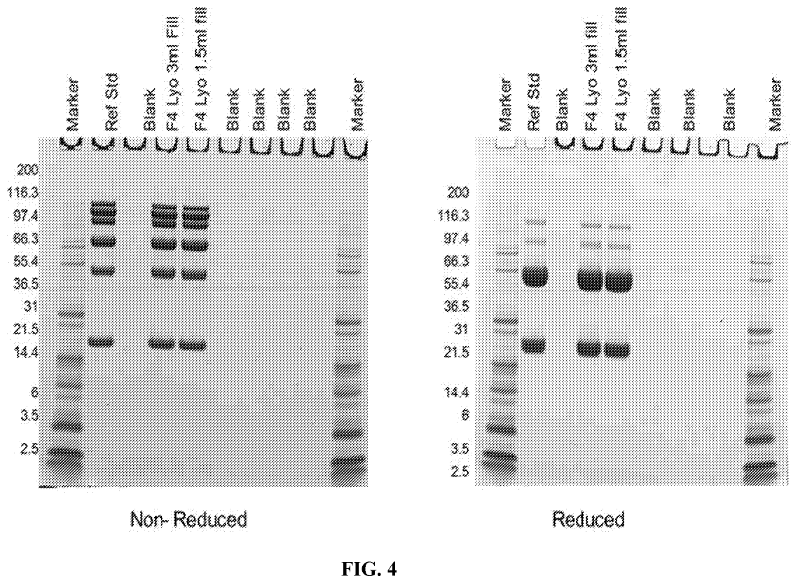

FIG. 4 shows the SDS-PAGE analysis for each fill volume, both reduced and non-reduced, as compared to the formulation standard. The results demonstrate that changing the fill volume did not have any impact on the SDS-PAGE profile.

Table 29 below summarizes the SEC-HPLC data, including the percentages of HMW peaks, main peaks and LMW peaks for samples lyophilized at each fill volume. Regardless of the fill volume evaluated, lyophilized samples behaved similarly and there was no difference in main peak areas measured before and after lyophilization. No changes were observed for either fill volume and that profiles were comparable to the 5 mL fill volume lyophilized in study described in Section 6.3.

TABLE 29

| | Peak Percentages (%) | | | | | | | |
|---|---|---|---|---|---|---|---|---|
| | Main Peak | | | | Peak Area (mAu) | | | |
| | Retention | | Main | Post | | Main | Post | |
| Sample Name | Time | Pre Peaks | Peak | Peaks | Pre Peaks | Peak | Peaks | Total |
| Reference standard | 19.8 | 1.4 | 95.2 | 3.4 | 429 | 29155 | 1045 | 30629 |
| F4, active, lyo, 3.0 mL fill | 19.8 | 1.3 | 95.4 | 3.3 | 481 | 34059 | 1174 | 35714 |
| F4, active, lyo, 1.5 mL fill | 19.8 | 1.4 | 95.1 | 3.5 | 487 | 32359 | 1198 | 34043 |

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

### 7. SEQUENCE LISTING

The present specification is being filed with a computer readable form (CRF) copy of the Sequence Listing. The CRF entitled “14369-244-228_SEQ_LISTING.txt”, which was created on Oct. 11, 2019 and is 39,693 bytes in size, is incorporated herein by reference in its entirety.

1-114. (canceled)

115. A pharmaceutical composition comprising
(a) an antibody drug conjugate comprising an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining region H1 (CDR H1), CDR H2, and CDR H3 in the heavy chain variable region set forth in SEQ ID NO: 7 and a light chain variable region comprising CDR L1, CDR L2, and CDR L3 in the light chain variable region set forth in SEQ ID NO: 8; and
(b) a pharmaceutically acceptable excipient comprising L-histidine in a range of 5 to 50 mM, polysorbate-20 in a range of 0.001 to 0.1% (v/v), sucrose in a range of 1 to 20% (w/v), and hydrochloric acid (HCl), wherein the pharmaceutical composition has a pH in a range of 5.5 to 6.5 at 15° C. to 27° C.

116. The pharmaceutical composition of claim 115, wherein:
(a) the antibody or antigen binding fragment thereof comprises CDR H1 comprising the amino acid sequence of SEQ ID NO: 9, CDR H2 comprising the amino acid sequence of SEQ ID NO: 10, CDR H3 comprising the amino acid sequence of SEQ ID NO: 11, CDR L1 comprising the amino acid sequence of SEQ ID NO: 12, CDR L2 comprising the amino acid sequence of SEQ ID NO: 13, and CDR L3 comprising the amino acid sequence of SEQ ID NO: 14;
(b) the antibody or antigen binding fragment thereof comprises a heavy chain variable region comprising the amino acid sequence ranging from the 20th amino acid (glutamic acid) to the 136th amino acid (serine) of SEQ ID NO: 7 and a light chain variable region comprising the amino acid sequence ranging from the 23rd amino acid (aspartic acid) to the 130th amino acid (arginine) of SEQ ID NO: 8; and/or
(c) the antibody comprises a heavy chain comprising the amino acid sequence ranging from the 20th amino acid (glutamic acid) to the 466th amino acid (lysine) of SEQ ID NO: 7 and a light chain comprising the amino acid sequence ranging from the 23rd amino acid (aspartic acid) to the 236th amino acid (cysteine) of SEQ ID NO: 8.

117. The pharmaceutical composition of claim 115, wherein:
(a) the antigen binding fragment is an Fab, F(ab′)₂, Fv, or scFv fragment;
(b) the antibody is a fully human antibody; and/or
(c) the antibody or antigen binding fragment thereof is recombinantly produced.

118. The pharmaceutical composition of claim 115, wherein the antibody drug conjugate has the following structure: 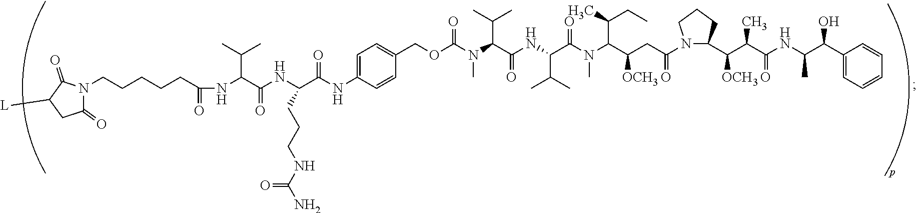
wherein L- represents the antibody or antigen binding fragment thereof and (i) p is from 1 to 10 or (ii) p is from 2 to 8.

119. The pharmaceutical composition of claim 115, wherein the antibody or antigen binding fragment thereof is linked to each unit of monomethyl auristatin E (MMAE) via a linker.

120. The pharmaceutical composition of claim 119, wherein the linker is an enzyme-cleavable linker, and wherein the linker forms a bond with a sulfur atom of the antibody or antigen binding fragment thereof.

121. The pharmaceutical composition of claim 119, wherein the linker has a formula of: -Aa-Ww-Yy-; wherein -A- is a stretcher unit, a is 0 or 1; —W— is an amino acid unit, w is an integer ranging from 0 to 12; and —Y— is a spacer unit, y is 0, 1, or 2.

122. The pharmaceutical composition of claim 121, wherein the stretcher unit has the structure of Formula (1) below; the amino acid unit is valine citrulline; and the spacer unit is a PAB group comprising the structure of Formula (2) below:

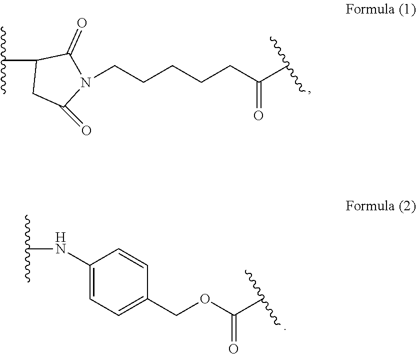 [SMILES: *C(=O)CCCCCN1C(=O)CC(*)C1=O ; *Nc1ccc(COC(*)=O)cc1]

123. The pharmaceutical composition of claim 121, wherein the stretcher unit forms a bond with a sulfur atom of the antibody or antigen binding fragment thereof, and wherein the spacer unit is linked to MMAE via a carbamate group.

124. The pharmaceutical composition of claim 115, comprising the antibody drug conjugate at a concentration of (i) from 1 to 20 mg/mL; (ii) from 5 to 15 mg/mL; (iii) from 8 to 12 mg/mL; or (iv) about 10 mg/mL.

125. The pharmaceutical composition of claim 115, wherein the L-histidine is present (i) in the range of 10 to 40 mM; (ii) in the range of 15 to 35 mM; (iii) in the range of 15 to 30 mM; (iv) in the range of 15 to 25 mM; or (v) at about 20 mM.

126. The pharmaceutical composition of claim 115, wherein the polysorbate-20 is present (i) in the range of 0.0025 to 0.075% (v/v); (ii) in the range of 0.005 to 0.05% (v/v); (iii) in the range of 0.01 to 0.03% (v/v); or (iv) at about 0.02% (v/v).

127. The pharmaceutical composition of claim 115, wherein the sucrose is present (i) in the range of 2 to 15% (w/v); (i) in the range of 3 to 10% (w/v); (i) in the range of 4 to 6% (w/v); or (ii) at about 5.0% (w/v).

128. The pharmaceutical composition of claim 115, wherein the pharmaceutical composition has a pH (i) in the range of 5.7 to 6.3, or (ii) about 6.0; and wherein the pH is taken (i) at 15° C. to 27° C., or (ii) at 25° C.

129. The pharmaceutical composition of claim 115, wherein the pH is adjusted by HCl.

130. The pharmaceutical composition of claim 115, comprising L-histidine in the range of 5 to 50 mM, polysorbate-20 in the range of 0.001 to 0.1% (v/v), sucrose in the range of 4 to 6% (w/v).

131. The pharmaceutical composition of claim 115, comprising about 20 mM L-histidine, about 0.02% (w/v) polysorbate-20, and about 5.0% (w/v) sucrose.

132. The pharmaceutical composition of claim 131, wherein the pH is 6.0 at room temperature or at 25° C.

133. The pharmaceutical composition of claim 131, wherein the antibody drug conjugate is at a concentration of about 10 mg/mL.

134. The pharmaceutical composition of claim 115, wherein:
(a) the pharmaceutical composition is in a liquid form or is lyophilized; or
(b) the pharmaceutical composition is stored at −80° C., 4° C., 25° C., or 37° C.

135. A lyophilized composition made by freeze-drying the pharmaceutical composition of claim 115.

136. A method of treating cancer in a human subject, comprising administering to the subject an effective amount of the pharmaceutical composition of claim 115, wherein the cancer has tumor cells expressing 191P4D12.

137. The method of claim 136, wherein the cancer is colon cancer, pancreatic cancer, ovarian cancer, lung cancer, bladder cancer, urothelial cancer, breast cancer, esophageal cancer, head cancer, neck cancer, or non-small cell lung cancer.

138. The method of claim 137, wherein the bladder cancer is advanced bladder cancer, advanced urothelial cancer, metastatic bladder cancer, or metastatic urothelial cancer.

139. The method of claim 136, further comprising administering to the subject a second therapeutic agent, wherein the second therapeutic agent is an immune checkpoint inhibitor.

140. The method of claim 139, wherein the immune checkpoint inhibitor is:
(a) a PD-1 inhibitor or a PD-L1 inhibitor;
(b) nivolumab; or
(c) selected from a group consisting of atezolizumab, avelumab, and durvalumab.

141. The method of claim 136, wherein the antibody drug conjugate in the pharmaceutical composition is administered at a dose of (i) 1 to 10 mg/kg of the subject's body weight; (ii) 1 to 5 mg/kg of the subject's body weight; (iii) 1 to 2.5 mg/kg of the subject's body weight; (iv) 1 to 1.25 mg/kg of the subject's body weight; or (v) about 1 mg/kg or about 1.25 mg/kg of the subject's body weight.

142. The method of claim 141, wherein the pharmaceutical composition is administered by an intravenous (IV) injection or infusion.

143. The method of claim 142, wherein the pharmaceutical composition is administered by an intravenous (IV) injection or infusion over about 30 minutes on Day 1 and 8 of every three-week cycle.

144. The method of claim 143, further comprising administering an immune checkpoint inhibitor by an intravenous (IV) injection or infusion on Day 1 of every three-week cycle, wherein the immune checkpoint inhibitor is administered in an amount of about 100 mg to about 1500 mg over about 30 minutes or 60 minutes.

145. The method of claim 142, wherein the pharmaceutical composition is administered by an intravenous (IV) injection or infusion over about 30 minutes on Days 1, 8 and 15 of every four-week cycle.

146. The method of claim 145, further comprising administering an immune checkpoint inhibitor by an intravenous (IV) injection or infusion.
